(19) United States
(12) Patent Application Publication
(10) Pub. No.: US 2026/0062455 A1
(43) Pub. Date: Mar. 5, 2026

(54) UPTAKE MECHANISM OF ESSENTIAL LYSOPHOSPHOLIPIDS INTO THE BRAIN AND INHIBITION BY ENDOGENOUS-RETROVIRAL ENVELOPE PROTEIN

(71) Applicant: INSTITUT PASTEUR, PARIS CEDEX 15 (FR)

(72) Inventors: Nicolas REYES, Bordeaux (FR); Maria MARTINEZ-MOLLEDO, Burgos (ES)

(21) Appl. No.: 18/833,578

(22) PCT Filed: Mar. 3, 2023
(86) PCT No.: PCT/IB2023/000127

§ 371 Date: Jul. 26, 2024

Related U.S. Application Data
(60) Provisional application No. 63/316,885, filed on Mar. 4, 2022.

(51) Int. Cl. C07K 14/705; A61K 38/00

(52) U.S. Cl. CPC C07K 14/705; A61K 38/00

(57) ABSTRACT

The disclosure provides the structure of an MFSD2A-SYNC2 complex together with functional data that revealed important molecular aspects of MFSD2A transport cycle, receptor-mediated cell-cell fusion, and pharmacology and resulted in the identification of two novel allosteric modulators of MFSD2A, which are two soluble fragments of SYNC2, namely SYNC2_{su-co} and SYNC2_{su-co-2}, representing first-in-class molecules to inhibit MFSD2A LPCs uptake and increase transcytosis rate.

### SEQUENCE LISTING

The instant application contains a Sequence Listing which has been submitted electronically in XML file format and is hereby incorporated by reference in its entirety. Said XML copy, created on May 16, 2025 is named D12021-40_SL.xml and is 121,742 bytes in size.

### FIELD

This disclosure is in the field of function of Major-facilitator superfamily containing 2A (MFSD2A) protein in blood brain barrier permeability and its selective allosteric inhibition by syncytin (SYNC).

### BACKGROUND

The brain selectively exchanges solutes with the blood through specialized blood-brain barrier (BBB). BBB integrity is essential to cognitive function, and prevents passage of neurotoxic molecules and pathogens, while it imposes major restrictions for delivery of therapeutic drugs into the brain (Chow and Gu, 2015; Zlokovic, 2008). MFSD2A is selectively enriched at BBB endothelium (Ben-Zvi et al., 2014; Nguyen et al., 2014), and constitutes the main uptake mechanism of docosahexaenoic acid (DHA) (Nguyen et al., 2014), an essential omega-3 fatty acid for brain and retina development, and function (Horrocks and Yeo, 1999; Kidd, 2007; Wong et al., 2016; Wong and Silver, 2020). MFSD2A transports esterified DHA in the form of lysophosphatidylcholine (LPC) in a Na+ dependent manner (Nguyen et al., 2014; Quek et al., 2016). The importance of MFSD2A transport function is highlighted by single-residue inactivating mutations in humans that cause severe microencephaly and intellectual disability (Alakbarzade et al., 2015; Guemez-Gamboa et al., 2015; Harel et al., 2018; Scala et al., 2020). Notably, mfsd2a knock-out and mutant mice show leaky BBB due to selective increase in caveolar transcytosis and reveal mfsd2a lipid transport as a key mechanism to control BBB permeability, as well as to normal BBB development and function (Andreone et al., 2017; Ben-Zvi et al., 2014; Chow and Gu, 2017; O'Brown et al., 2019). Consistently, downregulation of mfsd2a is associated to age-related increase in BBB permeability to neurotoxic proteins from plasma, and it could contribute to neurodegenerative diseases (Yang et al., 2020). Thus, MFSD2A has emerged as a drug target of paramount importance to increase BBB permeability, and aid delivering therapeutic molecules into the brain (Wang et al., 2016), but selective transport inhibitors that could play this role have not been reported.

MFSD2A transport cycle involves a series of conformational changes to bind and release LPC on opposite sides of the membrane, and recent cryo-electron microscopy (cryo-EM) structures of mouse (Mm-mfsd2a) (Cater et al., 2021) and chicken (Gg-mfsd2a) (Wood et al., 2021) orthologs offered a partial view of the cycle revealing open outward-facing, and partly-occluded inward-facing states, respectively. However, due to lack of structural information on additional and important intermediate states of the cycle, MFSD2A transport mechanism remains incompletely understood, particularly regarding how LPC is translocated across the membrane.

In the placenta, MFSD2A contributes to establish and maintain a radically different cellular blood barrier than that at the maternal-fetal interface. In humans, this interface is formed by fusion of cells into a multinucleated-epithelial layer, namely syncytiotrophoblast (ST), directly bathed in maternal blood (Robbins and Bakardjiev, 2012). Strikingly, cell-cell fusion is achieved by a retroviral mechanism and mediated by binding of envelope glycoproteins of retroviral origin, known as syncytin (SYNC), to cellular receptors (Blaise et al., 2003; Blond et al., 2000; Mallet et al., 2004; Mi et al., 2000; Roberts et al., 2021). SYNC2 is encoded by human endogenous retrovirus FDR (ERV-FDR), an extinct gamma-retrovirus that entered the simian lineage over 40 million years ago and is a homo-trimeric class-1 fusion protein that selectively uses MFSD2A as cellular receptor (Blaise et al., 2003; Esnault et al., 2008). Each SYNC2 protomer contains N-terminal surface subunit (SYNC2_{SU}) that binds MFSD2A, as well as C-terminal transmembrane subunit (SYNC2_{TM}) that conveys the membrane fusion machinery, including a transmembrane alpha-helix that anchors SYNC2 to the cell surface. Retroviral class-1 envelope fusion mechanism involves large conformational changes of the TM-subunit from so-called pre-fusion to post-fusion states (Harrison, 2005; Rey and Lok, 2018). In SYNC2, the post-fusion transition is somehow triggered by binding of SYNC2_{SU} to MFSD2A, and concomitant dissociation of the two subunits (Chen et al., 2008). X-ray crystal structures of SYNC2_{TM} ectodomain revealed a 6-helix bundle arrangement typical of gamma-retroviral post-fusion state, consistent with class-1 fusion mechanism (Renard et al., 2005; Ruigrok et al., 2019), but SYNC2_{SU} structure, and receptor-recognition mechanism remain unknown. In addition, no MFSD2A allosteric modulators have been reported to date.

### SUMMARY

MFSD2A lipid transport and MFSD2A-controlled caveolar transcytosis are key mechanisms to control BBB permeability. Selective transport inhibitors that could reversibly permeabilize the BBB and aid delivering therapeutic molecules into the brain are unknow. In this work, human MFSD2A mechanisms were investigated using single-particle cryo-electron microscopy (cryo-EM) and functional approaches. The structure of MFSD2A-SYNC2 complex is first described herein and, together with accompanying functional data, revealed important molecular aspects of MFSD2A transport cycle, receptor-mediated cell-cell fusion, and pharmacology. This approach resulted in the identification of two novel allosteric modulators of MFSD2A, which are two soluble fragments of SYNC2, namely SYNC2_{SU-CO} and SYNC2_{SU-CO-2}. SYNC2 and its soluble fragments represent first-in-class molecules to inhibit MFSD2A LPCs uptake and increase transcytosis rate. SYNC2 fragment conjugates with biologic therapies encompass a unique double mechanism to aid transport across the BBB (receptor-mediated and increase transcytosis mechanisms). Indeed, because MFSD2A controls caveolar transcytosis across the BBB, selective inhibitors of this transporter may facilitate passage of small compounds but also macromolecules (e.g. antibodies, DNA, RNA), and nanomaterials (e.g. liposomes) with genetic or other materials encapsulated within. As a complementary mechanism, transport across the BBB of drugs conjugated with SYNC2 fragments such as SYNC2_{SU-CO}, and SYNC2_{SU-CO-2} may also be facilitated through receptor-mediated mechanism.

In one embodiment, the disclosure provides an allosteric inhibitor of Major-facilitator superfamily containing 2A (MFSD2A) in the blood brain barrier, in particular an allosteric inhibitor comprising an isolated syncytin 2 (SYNC2) polypeptide fragment.

In one embodiment, the disclosure provides a promoter of MFSD2A-mediated blood brain barrier vesicular transcytosis, in particular a promoter comprising an isolated SYNC2 polypeptide fragment.

In one embodiment, the allosteric inhibitor of MFSD2A is a polypeptide, in particular is a polypeptide comprising an isolated SYNC2 polypeptide fragment.

In one embodiment, the promoter of MFSD2A-mediated blood brain barrier vesicular transcytosis is a polypeptide, in particular is a polypeptide comprising an isolated SYNC2 polypeptide fragment.

In one embodiment the polypeptide is not syncytin 2 (SYNC2) in particular is not human syncytin 2 (SYNC2_{WT}).

In one embodiment, the disclosure provides an isolated syncytin 2 (SYNC2) polypeptide fragment comprising, consisting, or consisting essentially of:
(i) a polypeptide of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(ii) a polypeptide whose sequence is at least 95%, at least 96%, at least 97%, at least 98%, or at least 99% identical to SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24, including polypeptides of SEQ ID NOs 22-24;
(iii) a polypeptide having 1, 2, 3, 4, 5, 6, 7, 8, 9, 10, or more amino acid substitutions, deletions, and or additions relative to an amino acid sequence selected from SEQ ID NOs 3-24, but still having at least 85% identity to SYNC2 (SEQ ID NO:3); or
(iv) a soluble fragment of any one of (i)-(iii), wherein the fragment is at least 20, 30, 40, 50, 60, 70, 80, 90, 100, 120, 140, 160, 180, 200, 220, 240, 260, 280, or at least 300 amino acid residues long but shorter than 350. In one embodiment, said polypeptide comprises, consists, or consists essentially of SYNC2_{SU-CO} (SEQ ID NO. 7), SYNC2_{SU-CO-2} (SEQ ID NO. 8) or SYNC2_{SU-CO-3} (SEQ ID NO: 9).

In one embodiment, the disclosure provides a SYNC2 polypeptide fragment that acts as an allosteric inhibitor of MFSD2A in the blood brain barrier and/or a promoter of MFSD2A-mediated blood brain barrier vesicular transcytosis, wherein the polypeptide fragment comprises, consists, or consists essentially of SYNC2_{SU-CO} (SEQ ID NO. 7), SYNC2_{SU-CO-2} (SEQ ID NO. 8), SYNC2_{SU-CO-3} (SEQ ID NO: 9), SYNC2_{SU-CO} (C43) (SEQ ID NO: 10), SYNC2_{SU-CO-2 (C43)} (SEQ ID NO: 11), SYNC2_{SU-CO-3 (C43)} (SEQ ID NO: 12), SYNC2_{SU-WT} (SEQ ID NO: 13), SYNC2_{SU-WT-2} (SEQ ID NO. 14), SYNC2_{SU-WT-3} (SEQ ID NO: 15), SYNC2_{SU-C43S} (SEQ ID NO: 16), SYNC2_{SU-C43S-2} (SEQ ID NO: 17), SYNC2_{SU-C43S-3} (SEQ ID NO: 18), RBL1 (SEQ ID NO: 19), RBL2 (SEQ ID NO: 20), RBL3 (SEQ ID NO: 21), RBLs4 (SEQ ID NO: 22), RBLs5 (SEQ ID NO: 24), or RBLs6 (SEQ ID NO: 24).

In one embodiment, the allosteric inhibitor of MFSD2A competes for binding to MFSD2A with the SYNC2 polypeptide or with one of the polypeptides of sequence of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24. In particular this allosteric inhibitor comprises or is a polypeptide. In particular, the polypeptide is not syncytin 2 (SYNC2) in particular is not human syncytin 2 (SYNC2).

In one embodiment, the promoter of MFSD2A-mediated blood brain barrier vesicular transcytosis competes for binding to MFSD2A with the SYNC2 polypeptide or with one of the polypeptides of sequence of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24. In particular this allosteric inhibitor comprises or is a polypeptide. In particular, the polypeptide is not syncytin 2 (SYNC2) in particular is not human syncytin 2 (SYNC2).

In one embodiment, said polypeptide binds MFSD2A with a K_{D} of at least about 1×10⁻⁵ M, at least about 1×10⁻⁶ M, 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

Also provided are polypeptide-drug conjugates, fusion proteins and nucleic acids encoding said SYNC2 polypeptide fragments, vectors and cells comprising the same; pharmaceutical compositions comprising said polypeptide-drug conjugates, fusion proteins, nucleic acids, vectors, and/or cells; methods of transiently inhibiting MFSD2A transport function in the blood brain barrier (BBB) in a subject in need thereof, comprising administering to the subject an effective dose of one or more of said polypeptide-drug conjugates, fusion proteins, nucleic acids, vectors, and/or cells or their pharmaceutical compositions; methods of transiently increasing vesicular transcytosis across BBB endothelium in a subject in need thereof, comprising administering to the subject an effective dose of one or more of said polypeptide-drug conjugates, fusion proteins, nucleic acids, vectors, and/or cells or their pharmaceutical compositions; and methods of transiently increasing BBB permeability to deliver therapeutic small-compounds, macromolecules (e.g. antibodies), nanomaterials (e.g. liposomes, gene therapy), into the brain of a subject in need thereof, comprising administering to the subject an effective dose of one or more of said polypeptide-drug conjugates, fusion proteins, nucleic acids, vectors, and/or cells or their pharmaceutical compositions.

In this application, the terms peptide and polypeptide are used interchangeably. The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs.

The compound which is an allosteric inhibitor of MFSD2A or a promoter of MFSD2A-mediated blood brain barrier vesicular transcytosis may be assessed using the methods disclosed below as a method to assay/screen for the capability to inhibit sodium-dependent uptake by MFSD2A or the methods described in the Examples, in particular the methods as disclosed in relation to FIG. 1A in order to determine whether it “competes for binding to MFSD2A”. Such assay may involve SYNC2 or SYNC2_{CO} or SYNC2_{SU} or SYNC2_{SU-CO} or a SYNC2 polypeptide fragment e.g., one of the herein disclosed specific fragments (in particular, RBL1, RBL2, RBL3) as a positive reference for binding to MFSD2A to determine the competition capability of the assayed compound. The competition capability of the compound assessed with one of the SYNC2 species disclosed above may be determined by the detection of competition to inhibit of the transport of MFSD2A substrate such as LPC or LPC analog such as Lyso NBD PC (FIG. 1A). Alternatively, the competition capability of the compound assessed with one of the SYNC2 species disclosed above may be determined by the detection of competition to promote the fusion of cell membranes that may be determined using a split-GFP complementation assay as described herein (FIG. 1B).

In one embodiment, the term “solubility” refers to the property of a SYNC2 polypeptide fragment or conjugate to dissolve in a liquid solvent and form a homogeneous solution. Solubility is typically expressed as a concentration, either by mass of solute per unit volume of solvent (g of solute per kg of solvent, g per dL (100 ml), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° ( ) or about body temperature (−37° C.). In certain embodiments, a p97 polypeptide or conjugate has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/ml at room temperature or at about 37° C.

In another embodiment, the disclosure provides a polypeptide-drug conjugate comprising one of said polypeptides covalently linked to a drug, directly or through a linker, wherein the drug is a small molecule (i.e. a drug molecular generally having a molecular weight less than about 1000 grams/mole, or less than about 750 grams/mole, or less than about 500 grams/mole), a macromolecule (e.g., an antibody, DNA, RNA), a liposome, or a nanoparticle. In another embodiment, the polypeptide is bound to a carrier (e.g., BSA). In one embodiment, the conjugate polypeptide is a “fusion protein” or “fusion polypeptide,” that is, a polypeptide that is created through the joining of two or more coding sequences, which originally coded for separate polypeptides; translation of the joined coding sequences results in a single, fusion polypeptide, typically with functional properties derived from each of the separate polypeptides. The linker may be physiologically stable or may include a releasable linker such as an enzymatically degradable linker (e.g., proteolytically cleavable linkers). In certain embodiments, the linker may be a peptide linker, for instance, as part of a SYNC2 fusion protein. In some embodiments, the linker may be a non-peptide linker or non-proteinaceous linker. In some embodiments, the linker may be particle, such as a nanoparticle

In one embodiment, the disclosure provides a nucleic acid encoding one or more of said polypeptides. In one embodiment, the nucleic acid comprises a sequence selected from SEQ ID NOs: 25-40. In one embodiment, the nucleic acid is DNA. In one embodiment, the nucleic acid is RNA. In one embodiment, the nucleic acid is modified RNA (mRNA). In one embodiment, the nucleic acid is delivered in a liposome or another nanoparticle, such as, without limitation, metal, gold, liposome, calcium phosphate with liposome, PEGylated metal such as gold, cationic, or nanoparticles made from or comprising the inventive DNA.

In one embodiment, the disclosure provides vectors or constructs, such as plasmids or retroviral constructs in which a DNA may be inserted, such as one encoding one or more of said polypeptides or comprising one or more of said nucleic acids. In one embodiment, viruses, which are useful as vectors include, but are not limited to, moloney murine leukemia virus (MMLV), adenovirus, adeno-associated virus (AAV), herpes simplex virus (HSV), lentiviruses, and spumaviruses. In one embodiment, viral-mediated gene transfer vehicles comprise vectors based on DNA viruses, such as adenovirus, adeno-associated virus and herpes virus, as well as retroviral based vectors.

In one embodiment, the disclosure provides a cell comprising one or more of said polypeptides, one or more of said polypeptide-drug conjugates, one or more of said nucleic acids, and/or one or more of said vectors.

In one embodiment, the disclosure provides a pharmaceutical composition comprising one or more of said polypeptides, one or more of said polypeptide-drug conjugates, one or more of said nucleic acids, one or more of said vectors, one or more of said cells, or combinations thereof, and a pharmaceutically acceptable carrier, diluent, or excipient. Pharmaceutical compositions containing one or more of the peptides described herein can be formulated in any conventional manner. Proper formulation is dependent in part upon the route of administration selected. Routes of administration include, but are not limited to parenteral (e.g., intravenous, intra-arterial, subcutaneous, rectal, subcutaneous, intramuscular), percutaneous, transmucosal, sublingual and intestinal administration. The compositions described herein can also comprise one or more pharmaceutically acceptable excipients and/or carriers. The pharmaceutically acceptable excipients and/or carriers for use in the compositions of the present invention can be selected based upon a number of factors including the particular compound used, and its concentration, stability and intended bioavailability; the subject, its age, size and general condition; and the route of administration. The peptides described herein may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces. These biologically active or inert agents can include, for example, enzyme inhibitors and absorption enhancers. In addition, various drug delivery agents may be included in the compositions to facilitate delivery of the peptides to their target. These drug delivery agents can comprise nanoparticles, microparticles, liposomes or others. The peptides can be covalently or non-covalently associated with the delivery vehicles via a linkage that may be suitably cleaved at the target

In one embodiment, the disclosure provides a modulator of MFSD2A membrane receptor inducing the outward-facing state. In one embodiment, the modulator comprises one or more of said polypeptides of the disclosure. In one embodiment, the polypeptide is administered as a soluble polypeptide. In one embodiment, the polypeptide is administered in a membrane-bound form at the surface of liposomes or other nanoparticles containing active drugs/therapeutic payloads for delivery into the brain. In one embodiment, the polypeptide is linked (directly or indirectly) to a therapeutic payload thus facilitating delivery of the therapeutic payload across the BBB.

In certain embodiments, the SYNC2 polypeptide sequence and the agent are each, individually or as a pre-existing conjugate, bound to or encapsulated within a particle, e.g., a nanoparticle, bead, lipid formulation, lipid particle, or liposome, e.g., immunoliposome. For instance, in particular embodiments, the SYNC2 polypeptide sequence is bound to the surface of a particle, and the agent of interest is bound to the surface of the particle and/or encapsulated within the particle. In some of these and related embodiments, the SYNC2 polypeptide and the agent are covalently or operatively linked to each other only via the particle itself (e.g., nanoparticle, liposome), and are not covalently linked to each other in any other way; that is, they are bound individually to the same particle. In other embodiments, the SYNC2 polypeptide and the agent are first covalently or non-covalently conjugated to each other, as described herein (e.g., via a linker molecule), and are then bound to or encapsulated within a particle (e.g., immunoliposome, nanoparticle). In specific embodiments, the particle is a liposome, and the composition comprises one or more SYNC2 polypeptides, one or more agents of interest, and a mixture of lipids to form a liposome (e.g., phospholipids, mixed lipid chains with surfactant properties). In some embodiments, the SYNC2 polypeptide and the agent are individually mixed with the lipid/liposome mixture, such that the formation of liposome structures operatively links the SYNC2 polypeptide and the agent without the need for covalent conjugation. In other aspects, the SYNC2 polypeptide and the agent are first covalently or non-covalently conjugated to each other, as described herein, and then mixed with lipids to form a liposome. The SYNC2 polypeptide, the agent, or the SYNC2-agent conjugate may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

In one embodiment, the disclosure provides a method of transiently inhibiting MFSD2A transport function in the blood brain barrier (BBB) in a subject in need thereof, comprising administering to the subject an effective dose of one or more of said polypeptides, said polypeptide-drug conjugates, or said pharmaceutical compositions. In one embodiment, the subject is not pregnant.

In another embodiment, the disclosure provides a method of transiently increasing vesicular transcytosis across BBB endothelium in a subject in need thereof, comprising administering to the subject an effective dose of one or more of said polypeptides, said polypeptide-drug conjugates, or said pharmaceutical compositions. In one embodiment, the subject is not pregnant.

In yet another embodiment, the disclosure provides a method of transiently increasing BBB permeability to deliver therapeutic small-drug/compounds, macromolecules (e.g. antibodies), nanomaterials (e.g. liposomes, gene therapy), into the brain of a subject in need thereof, comprising administering to the subject an effective dose of one or more of said polypeptides, said polypeptide-drug conjugates, or said pharmaceutical compositions. In one embodiment, the subject is not pregnant.

In one embodiment, the disclosure provides the use of one or more of said polypeptides, said polypeptide-drug conjugates, or said pharmaceutical compositions to transiently inhibit MFSD2A transport function in the BBB, transiently increase vesicular transcytosis across human BBB endothelium, or transiently increase BBB permeability to deliver therapeutic small-compounds, macromolecules, or nanomaterials to the brain of a subject in need thereof.

In one embodiment, the transient inhibition of MFSD2A transport function in the BBB, transient increase in vesicular transcytosis across human BBB endothelium, or transiently increase in BBB permeability to deliver therapeutic small-compounds, macromolecules, or nanomaterials to the brain has a duration of 1-60 minutes, preferably 1-10 minutes. In one embodiment, the subject has a neurological disorder.

In one embodiment, the terms “modulating/modulator” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount produced by no composition (e.g., the absence of polypeptide of conjugate of the invention) or a control composition, sample or test subject. In other embodiments, the BBB permeability and/or transport are increased by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to baseline BBB permeability or transport of the drug without the use of the polypeptide of the disclosure. “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between. As one non-limiting example, a control could compare the activity, such as the amount or rate of transport/delivery across the blood brain barrier, the rate and/or levels of distribution to central nervous system tissue, and/or the Cmax for plasma, central nervous system tissues, or any other systemic or peripheral non-central nervous system tissues, of a SYNC2-agent conjugate relative to the agent alone. By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The polypeptides may be administered in an effective amount, which will vary depending upon a variety of factors including the activity of the specific compound (e.g., conjugate) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, an effective daily dose is (for a 70 kg mammal) from about 0.001 mg/kg to about 100 mg/kg preferably a therapeutically effective dose is (for a 70 kg mammal) from about 0.01 mg/kg to about 50 mg/kg; more preferably an effective dose is (for a 70 kg mammal) from about 1 mg/kg (i.e., ˜70 mg) to about 25 mg/kg.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee.

FIG. 1: MFSD2A-SYNC2 functional and structural analyses. A, GFP fluorescence arising from fusion of cells expressing MFSD2A_{CO}, or MFSD2A_{WT} with those expressing SYNC2 constructs. B, Fluorescent substrate analog (LPCNBD) uptake in cells expressing MFSD2A constructs or negative control (excitatory amino acid transporter, EAAT1) using sodium-based (orange) and choline-based (purple) buffers, respectively. C, SYNC2_{CO-C43S} binding to MFSD2A_{CO} in non-denaturing detergent solutions. Solid line is the fit of a binding quadratic equation. D, Example micrograph depicting extraction box of 3:3 MFSD2A_{CO}:SYNC2_{CO} (red) and 1:1 MFSD2A_{CO}:SYNC2_{SU-CO} complexes (yellow), respectively. E, Representative 2D classes and 3D reconstruction of 3:3 MFSD2A_{CO}:SYNC2_{CO} complex. F, Representative 2D classes and refined 3D cryo-EM map of 1:1 MFSD2A_{CO}:SYNC2_{SU-CO} complex. Density corresponding to MFSD2A_{CO} (orange), SYNC2_{SU-CO} (green), and lipid/detergent molecules (grey), respectively, is shown. Plots (A-C) depict average of three independent experiment measurements, and error bars represent s.e.m. Circles represent values from individual experiments (A,B), and average values (C), respectively.

FIG. 2: Partially-occluded outward-facing structure of MFSD2A_{CO}. A, Membrane view of MFSD2A_{CO}-SYNC2_{SU-CO} complex. N- (blue) and C-domains (orange) form an outward-facing cavity in MFSD2A_{CO} displaying a clusters of conserved polar residues (yellow), and hydrophobic (gray) residues. B, Extracellular view of MFSD2A_{CO} with ECL3 atop the central cavity with conserved polar residues (yellow) at the bottom. SYNC2_{SU-CO} was removed for clarity of display. Close-up membrane view of conserved residues (yellow) is shown in inset. C, MFSD2A_{CO} solvent-accessible surface colored by residue hydrophobicity shows central and lateral cavities connected through a construction around F65. SYNC2_{SU-CO} was removed for clarity of display. D, Human disease-causing MFSD2A mutations (alpha-carbon, black sphere) are mapped on MFSD2A_{CO} structure. Conserved residues in the central (yellow), and lateral (gray) cavities are shown for reference.

FIG. 3: SYNC2_{SU-CO} structure and N-glycosylation. A, SYNC2_{SU-CO} structure displaying core (gold) and RBD (green). MFSD2A_{CO} is omitted for clarity of display. All seven predicted N-glycosylation sites display glycans (pink). B, ˜180° rotation along SYNC2_{SU-CO} long axis from (A) showing the side where glycans concentrate on SYNC2_{SU-CO} surface. C, Glycan N177 interacts with residues (blue) in the core. D, Glycan N312 interacts extensively with the RBD in close proximity to MFSD2A_{CO} (not displayed).

FIG. 4: Binding interface at MFSD2A_{CO}-SYNC2_{SU-CO} complex and transport inhibition. A, RBL1 (green), RBL2 (pink), and RBL3 (yellow) bind shallow crevices formed between ECLs of MFSD2A_{CO}, and are stabilized by N312 glycan and contacts with lipid/detergent molecules within the membrane plane. B, RBL1 interface. C, RBL2 interface. D, RBL3 interface. E, Fusogenic activity of cells expressing MFSD2A_{CO} and those expressing one of the following SYNC2 constructs: SYNC2_{CO}, C43S mutant (Co-C43S), M86A/N90A mutant (RBL1), N164A/Q165A mutant (RBL2), and E274A/F275A/F276A mutant (RBL3). F, SYNC2_{SU-CO} binding abolishes sodium-induced uptake of LPCNBD. Solid line is the fit of a Hill equation. Plots (E,F) depict average of three independent experiment measurements, and error bars represent s.e.m. Circles represent values from individual experiments (E), and average values (F), respectively.

FIG. 5: MFSD2A_{CO} transport and SYNC2_{CO} allosteric inhibition mechanisms. Cartoon representation of MFSD2A_{CO} “rock-and-swing” transport mechanism, and SYNC2_{SU-CO} inhibition. Red and blue solid circles represent D93/E155 and R90/K436 charges, respectively, on MFSD2A_{CO} central cavity, while phosphate (P) and trimethyl-ammonium (N) groups of LPC are represented by empty red and blue circles. Co-transported Na+ is represented as smaller empty circle. SYNC2_{SU-CO} (green) trapping a partially-occluded outward-facing state of the cycle is shown with N312 glycan in purple.

FIG. 6: Thermal stability comparison of wild-type and consensus designs. Detergent solubilized protein samples were pre-heated at 4, 45 and 70° C., and assayed by Fluorescence-based SEC. Traces corresponding to samples pre-heated at 45° C. are highlighted for comparison. A, MFSD2A_{WT}. B, MFSD2A consensus design. C, SYNC2_{WT}. D, SYNC2 consensus design. E, Representative SEC traces of purified MFSD2A_{CO}, SYNC2_{CO}, and MFSD2A_{CO}-SYNC2_{CO} complex, respectively, with the corresponding elution volumes in a SEC Superose6 column (24 ml bed volume).

FIG. 7: Cryo-EM data processing pipeline. A, Representative EM micrograph. B, Gallery of representative 2D class-averages. C, 3D classes from heterogenous refinement. D, Non-uniform refinement 3D map. E, Local-refinement map after micelle removal. F, Viewing direction distribution plot. G, Color coded map according to local resolution estimation. H, Fourier shell correlation (FSC) plot of local refinement with FSC threshold at 0.143 and 0.5.

FIG. 8: EM density in MFSD2A_{CO}-SYNC2_{SU-CO} structure. Cryo-EM density corresponding to representative individual structural elements of MFSD2A_{CO} and SYNC2_{SU-CO}.

FIG. 9: Amino acid sequence alignment of MFSD2A and MFSD2B mammalian orthologs. Asterisks indicate the position of MFSD2A conserved residues that potentially contribute to LPC (black) and Na+ (green) coordination in the central cavity. Solid circles indicate conserved residues facing the lateral cavity, and empty circles those forming the constriction. Yellow polygons indicate cysteine residues involved in ECL3-ECL6 disulfide bond. Dashed lines indicate MFSD2A regions that were not modeled in the structure, and red arrows potential N-glycosylation sites on MFSD2A.

FIG. 10: Cavity analysis of representative MFS transporters. MFS transporters are represented with N-domain pink and C-domain cyan. Total cavity surface (central+lateral) was calculated with CASTp 3.0 and is represented in grey. Contribution of the lateral cavity to the total volume is indicated underneath the structures, along with the PDB IDs.

FIG. 11: Structural comparison between MFSD2A_{CO} outward- and Gg-mfsd2a inward-facing states. Cartoon, and solvent-accessible surface colored by residue hydrophobicity representations of Mm-mfsd2a (left, PDB 7N98), MFSD2A_{CO} (center), and Gg-mfsd2a (right, PDB 7MJS) structures. Central and lateral cavities are highlighted to show changes during the transport cycle. Constrictions around F65 in MFSD2A, and the equivalent residues are also shown.

FIG. 12: Amino acid sequence alignment of SYNC2_{SU-CO} simian orthologs. Disulfide bonds are indicated by red circles and numbered as follows: S1 corresponds to disulfide C126-C145, S2 to C200-C253, S3 to C291-C308, and S4 to C301-C317. Glycan molecules are represented over corresponding asparagine residues with N-Acetylglucosamine (blue square), and mannose (green circle) residues indicated. Receptor binding loops (RBLs) are framed in red squares. In SYNC2 consensus design the following exchanges were introduced: M128T, S153P, T190S, F486L and T4871. Exchanges that localize to SYNC2_{SU-CO} are marked with asterisks.

FIG. 13: MFSD2A amino acid conservation surface mapping. Amino acid conservation across MFSD2A vertebrate orthologs is mapped into the structure of MFSD2A_{CO}-SYNC2_{CO} complex. SYNC2_{SU-CO} is shown in gray and binds to poorly-conserved residues on the extracellular surface of MFSD2A_{CO}. Spheres represent alpha-carbon atoms of consensus exchanges introduced in MFSD2A_{WT} to improve thermal stability: V431, L51 V, L102F, P108S, C111R, M157L, T177S, K240N, V250A, C251S, 1254V, 1261T, 1279M, A280S, Y281F, T299A, V316A, 1383V, A386V, F420S, N471K, M491L.

### DETAILED DESCRIPTION

Brain and BBB development and function require uptake of docosahexaenoic omega-3 fatty acid in the form of lysophosphatidylcholine. MFSD2A is its main uptake route, and an important pharmaceutical target to facilitate passage of therapeutic molecules across the BBB. MFSD2A also functions as receptor of SYNC2 in human placenta, where it mediates cell-cell fusion and formation of maternal-fetal interface. Here, the disclosure provides novel information related to human MFSD2A transport and receptor mechanisms, as well as the cryo-electron microscopy structure of MFSD2A-SYNC2 complex. The disclosure provides that the structure reveals an elusive MFSD2A outward-facing state of the transport cycle, and an adapted alternating-access uptake mechanism for lipid substrates. The inventors further demonstrate that SYNC2 establishes an extensive binding-interface with MFSD2A and acts as long-sought potent inhibitor of MFSD2A transport. This disclosure provides new uncovered molecular mechanisms important to brain and placenta development and function. The unexpected interlink between MFSD2A transport and receptor mechanisms suggests strategies to aid delivering therapeutic macromolecules across the BBB.

In one embodiment, the structural and functional analyses of human MFSD2A_{CO}-SYNC2_{SU-CO} complex described in the Examples section revealed three key molecular aspects of MFSD2A function and pharmacology.

### Lipid Uptake Mechanism Across the BBB

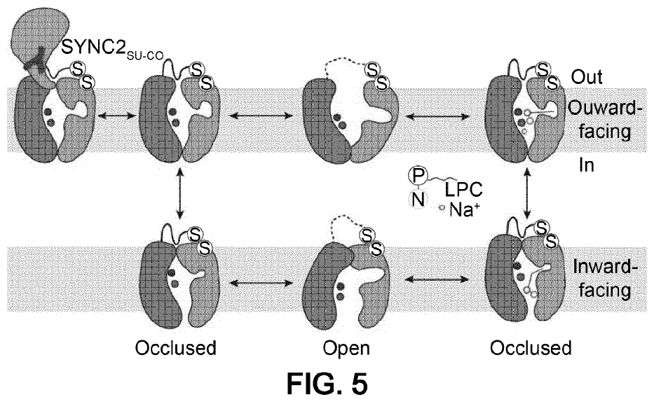

The inventors uncovered an elusive outward-facing state of MFSD2A transport cycle that reveals a large C-domain cavity, and propose an adapted “rocker-switch” transport mechanism, called “rock-and-swing,” to take up LPC across the BBB (FIG. 5). In this mechanism, outward-facing MFSD2A occludes headgroup and acyl-chain of LPC in central and lateral cavities, respectively. In the central cavity, charged-residue pairs R90/K436 and D93/E155 provide counter charges for coordination of zwitterionic headgroup, while the hydrophobic constriction around F65 “clamps” the lipid-tail and stabilizes it inside the lateral cavity. Rotation of N- and C-domains isomerizes the transporter into the inward-facing state orienting central and lateral cavities towards the cytoplasm, as observed in Gg-mfsd2a structure (Cater et al., 2021). Upon isomerization, the headgroup of LPC “swings” from the central-residue cluster into the cytoplasmic side of the transporter before its release, while the acyl-chain remains bound to the hydrophobic constriction and lateral cavity. We refer to this as “rock-and-swing” mechanism to differentiate from the classical “rocker-switch” reported for transport of smaller soluble substrates. The proposed mechanism explains MFSD2A substrate selectivity for substrates with zwitterionic headgroups and long fatty-acid tails (at least 14 C atoms) (Nguyen et al., 2014; Quek et al., 2016), since LPC headgroup charges match basic- and acidic-residue pairs in the central cavity, while the long acyl-chain is required to reach the constriction and lateral cavity for occlusion.

Accordingly, the disclosure provides that the mechanism of MFSD2A lipid transport is different (“rock-and-swing”) from the classical “rocker-switch” reported for transport of smaller soluble substrates. This information reveals a different target for the design of small and large molecule drugs that could be transported across the BBB by MFSD2A, used to inhibit MFSD2A, or both.

### SYNC2 Receptor-Recognition Mechanism

Highly-conserved SYNC2 orthologs are present in all simian species, and constitute a “fossil” record of what, more than 40 million years ago, was the spike of an exogenous infectious virus with variable receptor-binding determinants (Blaise et al., 2003). Different mammalian lineages express syncytin envelopes with unrelated receptor usage for placentation, reflecting independent captures of different ancient retroviruses (Blaise et al., 2003; Lavialle et al., 2013). The disclosed MFSD2A_{CO}-SYNC2_{SU-CO} complex constitutes the first structural characterization of a “fossilized” syncytin receptor-recognition mechanism, and reveals extensive interactions of receptor-binding loops (RBLs) with divergent residues on MFSD2A surface, further stabilized by direct contacts with lipid/detergent molecules, and surface glycans. Lack of MFSD2A amino acid conservation at SYNC2_{SU-CO} binding interface is consistent with independent viral captures across mammalian lineages. In turn, direct interactions of SYNC2_{SU-CO}-receptor-binding domain (RBD) with the membrane contrasts with reported receptor-recognition mechanisms of extant human retrovirus. The RBD of EBOV (Gong et al., 2016) and SARS-cov-2 viruses (Yan et al., 2020) envelopes bound to the respective receptors lay far from the membrane, while that of human immune deficiency virus (HIV) reaches the membrane plane, but it is shielded from lipids within the co-receptor binding pocket (Shaik et al., 2019).

The involvement of surface glycan molecules in retroviral receptor-mediated fusion mechanisms has been previously observed in HIV (Ozorowski et al., 2017; Shaik et al., 2019). Conservation of SYNC2 “glycan coat” during simian endogenization was likely important to preserve MFSD2A recognition mechanism, and/or the molecular events that trigger SYNC2_{TM} post-fusion transition upon receptor binding, as suggested by the intimate structural engagement of glycan N312 with the RBD, as well as glycosylation knockout mutants (Cui et al., 2016).

Accordingly, the disclosure provides a new model for the study of MFSD2A interactions with SYNC2 and lipids. This model can be used for rational drug design of inhibitors of MFSD2A that may be used to improve drug delivery across the BBB.

### Therapeutic-Drug Delivery Across the BBB

Neurological and neurodegenerative diseases affecting the central nervous system (CNS) remain a tremendous human health burden (Collaborators, 2019), and the BBB constitutes a major obstacle to deliver therapeutics into the CNS (Terstappen et al., 2021). MFSD2A has emerged as unique pharmacological target to overcome that obstacle (Wang et al., 2016), and the structural and functional results provided herein should aid to unleash its pharmacological potential. The structure of outward-facing partially-occluded MFSD2A may facilitate rational design of allosteric modulators and/or conjugated substrates with small-compound drugs. More importantly, the inventors demonstrate that soluble SYNC2_{SU-CO} inhibits MFSD2A transport function with sub-μM IC50. This suggests the exciting possibility to use SYNC2_{SU-CO} to transiently increase vesicular transcytosis across human BBB endothelium, and aid delivering therapeutic macromolecules and nano-materials. In addition, transport of drugs across the BBB may also be facilitated through conjugation with soluble SYNC2 fragments and receptor-mediated transport of the same via MFSD2A.

Accordingly, in one embodiment, the disclosure provides allosteric modulators of the (human) MFSD2A membrane receptor inducing outward-facing state. In one embodiment, the modulator comprises, or consists/consists essentially of a fragment of SYNC2 (SEQ ID NO:5) (isolated, purified or produced through recombinant technologies) or of SEQ ID NOs: 3, 4, or 6 (isolated, purified or produced through recombinant technologies). In one embodiment, the modulator comprises, or consists/consists essentially of, a soluble fragment of SEQ ID Nos 3-21 or of other syncytin (isolated, purified or produced through recombinant technologies). In one embodiment, the soluble fragment comprises SYNC2_{SU-CO}(SEQ ID NO: 7) (isolated, purified or produced through recombinant technologies). In one embodiment, the soluble fragment comprises SYNC2_{SU-CO-2} (SEQ ID NO: 8) (isolated, purified or produced through recombinant technologies). In one embodiment, the soluble fragment comprises SYNC2_{SU-CO-3} (SEQ ID NO: 9) (isolated, purified or produced through recombinant technologies). In one embodiment, the soluble fragment comprises SYNC2_{SU-CO}(c43) (SEQ ID NO: 10) (isolated, purified or produced through recombinant technologies). In one embodiment, the soluble fragment comprises SYNC2_{SU-CO-2} (c43) (SEQ ID NO: 11) (isolated, purified or produced through recombinant technologies). In one embodiment, the soluble fragment comprises SYNC2_{SU-CO-3} (c43) (SEQ ID NO: 12) (isolated, purified or produced through recombinant technologies). In one embodiment, the soluble fragment comprises SYNC2_{SU-WT} (SEQ ID NO: 13) (isolated, purified or produced through recombinant technologies). In one embodiment, the soluble fragment comprises SYNC2_{SU-WT-2} (SEQ ID NO. 14) (isolated, purified or produced through recombinant technologies). In one embodiment, the soluble fragment comprises SYNC2_{SU-WT-3} (SEQ ID NO: 15) (isolated, purified or produced through recombinant technologies). In one embodiment, the soluble fragment comprises SYNC2_{SU-C43S} (SEQ ID NO: 16) (isolated, purified or produced through recombinant technologies). In one embodiment, the soluble fragment comprises SYNC2_{SU-C43S-2} (SEQ ID NO: 17) (isolated, purified or produced through recombinant technologies). In one embodiment, the soluble fragment comprises SYNC2_{SU-C43S-3} (SEQ ID NO: 18) (isolated, purified or produced through recombinant technologies). In one embodiment, the soluble fragment comprises (SYNC2) RBL1 (SEQ ID NO: 19) (isolated, purified or produced through recombinant technologies). In one embodiment, the soluble fragment comprises (SYNC2) RBL2 (SEQ ID NO: 20) (isolated, purified or produced through recombinant technologies). In one embodiment, the soluble fragment comprises (SYNC2) RBL3 (SEQ ID NO: 21) (isolated, purified or produced through recombinant technologies). In one embodiment, the modulator comprises, or consists/consists essentially of, a synthetic fragment of SYNC2 comprising, consisting/consisting essentially of SEQ ID Nos 22-24 (isolated, purified or produced through recombinant technologies). In one embodiment, the synthetic fragment comprises RBLs4 (SEQ ID NO: 22) (isolated, purified or produced through recombinant technologies). In one embodiment, the synthetic fragment comprises RBLs5 (SEQ ID NO: 23) (isolated, purified or produced through recombinant technologies). In one embodiment, the synthetic fragment comprises RBLs6 (SEQ ID NO: 24) (isolated, purified or produced through recombinant technologies).

As used herein, the term “isolated” when used in reference to a polypeptide of the invention is intended to mean that the polypeptide is in a form that is relatively free from material that normally is associated with the nucleic acid or polypeptide in a cell, tissue or in a crude preparation. Therefore, an isolated polypeptide of the invention has been separated from one or more other components and is in a form that it is not normally found in nature. Generally, an isolated polypeptide will be in a substantially purified form, but also can include impure preparations such as preparations that enrich for the polypeptide so long as some materials or components normally associated with the molecule have been removed.

In one embodiment, said modulator may be at least 95%, at least 96%, at least 97%, at least 98%, or at least 99% identical to an amino acid sequence selected from SEQ ID Nos: 3 through 24. In one embodiment, said modulator has 1, 2, 3, 4, 5, 6, 7, 8, 9, 10, or more amino acid substitutions, deletions, and or additions relative to an amino acid sequence selected from list SEQ ID Nos 3-24. In one embodiment, the modulator furthermore comprises a polypeptide that is designed with reference to/based on the cryo-EM structures disclosed herein.

In one embodiment, said modulator has 1, 2, 3, 4, 5, 6, 7, 8, 9, 10, or more amino acid substitutions, deletions, and or additions relative to an amino acid sequence selected from SEQ ID NOs 3-24, but it still has at least 85% homology to SYNC2. In one embodiment, one or more of the substitutions is conservative. In one embodiment, one or more of the substitutions is not conservative. In one embodiment, said modulator comprises a signal peptide/signal sequence.

In one embodiment, said modulator is a soluble fragment of SEQ ID NOs: 3-24 isolated, purified or produced through recombinant technologies.

In one embodiment, the modulator binds MFSD2A with a K_{D} of at least about at least about 1×10⁻⁵ M, at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

In one embodiment, the fragment is at least 10, 20, 30, 40, 50, 60, 70, 80, 90, 100, 120, 140, 160, 180, 200, 220, 240, 260, 280, or at least 300 amino acid residues long but shorter than 350.

Also provided are nucleic acids encoding one or more of the polypeptides disclosed herein. Vectors comprising the nucleic acids are also provided as are recombinant cells comprising the vectors.

The term “nucleic acid” refers to a polymer of nucleotides (oligonucleotide or polynucleotide), wherein the nucleotides may be ribonucleotides, deoxyribonucleotides, modified nucleotides or mixtures thereof and may further include modified internucleotide linkages and/or modified 5′ and/or 3′ termini. The nucleotide sequence of the polymer may be chemically modified or artificial. Nucleic acids include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate (PS) linkage may be used. 2′-modified nucleotide (O-methyl, -O-methoxyethyl, and others) may be used. Other deoxynucleotide analogs include methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the disclosure. In one embodiment, the nucleic acid is a modified RNA or based-modified RNA encoding one or more of the polypeptides of the disclosure. Nucleic acids are usually synthesized using any of a variety of well-known enzymatic, recombinant DNA or chemical methods. In one embodiment, the nucleic acid comprises a codon optimized sequence encoding any one of the polypeptides of the disclosure. In one embodiment, the codon-optimized sequences are selected from SEQ ID NOs: 25-40. The term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein. In certain embodiments, a nucleic acid according to the present disclosure is codon optimized for expression in a non-human host cell. In other embodiments, the nucleic acid is codon optimized for the polypeptide's producer cell.

In one embodiment, the disclosure provides pharmaceutical compositions comprising one or more polypeptides of the disclosure, nucleic acids of the disclosure, vectors of the disclosure, and/or cells of the disclosure and a pharmaceutically acceptable excipient. Pharmaceutically acceptable compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition may also include any of a variety of stabilizing agents, such as an antioxidant for example. When the pharmaceutical composition includes a polypeptide, the polypeptide may be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition may also be complexed with molecules that enhance their in vivo attributes. Such molecules include, for example, carbohydrates, polyamines, amino acids, other peptides, ions (e.g., sodium, potassium, calcium, magnesium, manganese), and lipids.

Any of the polypeptides described herein can be prepared using standard methods in the art. For example, the peptides can be chemically synthesized via standard solid phase peptide synthesis or produced recombinantly (e.g., polypeptides, peptide aptamers). In addition, peptides can be chemically synthesized with D-amino acids, b2-amino acids, b3-Mhího acids, homo amino acids, gamma amino acids, peptoids, N-methyl amino acids, and other non-natural amino acid mimics and derivatives. In various embodiments, the peptides provided herein may be modified to improve deliverability, stability (e.g., cyclization, secondary structure formation, oxidation, hydrolysis, sequence deletions, lipidation) and/or potency, and to reduce degradation (e.g., cyclization, acetylation, amidation, D-amino acid replacement, peptoids, hydrocarbon stapling) or any other property important for drug delivery. The peptides may be modified by either natural processes, such as posttranslational processing, or by chemical modification techniques that are well known in the art. Modifications can occur anywhere in a peptide, including the peptide backbone, the amino acid side-chains and the amino or carboxyl termini. The same type of modification may be present in the same or varying degrees at several sites in a peptide. Also, a peptide may contain many types of modifications. Peptides may be branched, for example, as a result of ubiquitination, and they may be cyclic, with or without branching. Cyclic, branched, and branched cyclic polypeptides may result from posttranslation natural processes or may be made by synthetic methods. Modifications further include stapling, acetylation, acid addition, acylation, ADP-ribosylation, aldehyde addition, alkylamide addition, amidation, amination, biotinylation, carbamate addition, chloromethyl ketone addition, covalent attachment of a nucleotide or nucleotide derivative, cross-linking, cyclization, disulfide bond formation, demethylation, ester addition, formation of covalent cross-links, formation of cysteine-cysteine disulfide bonds, formation of pyroglutamate, formylation, gamma-carboxylation, glycosylation, GPI anchor formation, hydrazide addition, hydroxyamic acid addition, hydroxylation, iodination, lipid addition, methylation, myristoylation, oxidation, PEGylation, proteolytic processing, phosphorylation, prenylation, palmitoylation, addition of a purification tag, pyroglutamyl addition, racemization, selenoylation, sulfonamide addition, sulfation, transfer-RNA mediated addition of amino acids to proteins such as arginylation, ubiquitination, and urea addition. In one embodiment, the polypeptide is acetylated (e.g., the N-terminal amino acid residue is acetylated (COCH₃ or Ac-)). In one embodiment, the polypeptide is amidated. In one embodiment, the C-terminal amino acid residue is amidated (—NH2). Additionally, peptides of the disclosure may include several salt forms including hydrochloride salts, acetate salts, TFA salts, and sodium chloride salts.

Using known methods of protein engineering and recombinant DNA technology (including library screenings), variants may be generated to improve or alter the characteristics of the peptides described herein. Such variants include deletions, insertions, inversions, repeats, duplications, extensions, and substitutions (e.g., conservative substitutions and/or substitutions with nonstandard amino acids) selected according to general rules well known in the art so as have little effect on activity or improve activity. Positional libraries may be used in such methods. Variants may be selected from either chemical or DNA-encoded platforms. In some embodiments, the peptides are retro-inverso analogues of the peptides identified above, which are peptides composed of D-amino acids introduced in the sequence in reverse direction.

The disclosure also provides methods to transiently inhibit MFSD2A transport function in the BBB in a subject in need thereof. In one embodiment, the subject is not pregnant. In one embodiment, MFSD2A transport function in the BBB is inhibited by administration of an effective dose of one or more of the polypeptides of the disclosure, which are modulators of MFSD2A activity or function. In one embodiment, the modulator comprises, or consists/consists essentially of a fragment of SYNC2 (SEQ ID NO:5) (isolated, purified or produced through recombinant technologies) or of SEQ ID NOs: 3, 4, or 6 (isolated, purified or produced through recombinant technologies). In one embodiment, the modulator comprises, or consists/consists essentially of, a soluble fragment of SEQ ID Nos 3-24 or other syncytin (isolated, purified or produced through recombinant technologies). In one embodiment, the soluble fragment comprises SYNC2_{SU-CO} (SEQ ID NO. 7) (isolated, purified or produced through recombinant technologies). In one embodiment, the soluble fragment comprises SYNC2_{SU-CO-2} (SEQ ID NO. 8) (isolated, purified or produced through recombinant technologies). In one embodiment, the soluble fragment comprises SYNC2_{SU-CO-3} (SEQ ID NO: 9) (isolated, purified or produced through recombinant technologies). In one embodiment, the soluble fragment comprises SYNC2_{SU-CO} (c43) (SEQ ID NO: 10) (isolated, purified or produced through recombinant technologies). In one embodiment, the soluble fragment comprises SYNC2_{SU-CO-2 (C43)} (SEQ ID NO: 11) (isolated, purified or produced through recombinant technologies). In one embodiment, the soluble fragment comprises SYNC2_{SU-CO-3 (C43)} (SEQ ID NO: 12) (isolated, purified or produced through recombinant technologies). In one embodiment, the soluble fragment comprises SYNC2_{SU-WT} (SEQ ID NO: 13) (isolated, purified or produced through recombinant technologies). In one embodiment, the soluble fragment comprises SYNC2_{SU-WT-2} (SEQ ID NO. 14) (isolated, purified or produced through recombinant technologies). In one embodiment, the soluble fragment comprises SYNC2_{SU-WT-3} (SEQ ID NO: 15) (isolated, purified or produced through recombinant technologies). In one embodiment, the soluble fragment comprises SYNC2_{SU-C43S} (SEQ ID NO: 16) (isolated, purified or produced through recombinant technologies). In one embodiment, the soluble fragment comprises SYNC2_{SU-C43S-2} (SEQ ID NO: 17) (isolated, purified or produced through recombinant technologies). In one embodiment, the soluble fragment comprises SYNC2_{SU-C43S-3} (SEQ ID NO: 18) (isolated, purified or produced through recombinant technologies). In one embodiment, the soluble fragment comprises (SYNC2) RBL1 (SEQ ID NO: 19) (isolated, purified or produced through recombinant technologies). In one embodiment, the soluble fragment comprises (SYNC2) RBL2 (SEQ ID NO: 20) (isolated, purified or produced through recombinant technologies). In one embodiment, the soluble fragment comprises (SYNC2) RBL3 (SEQ ID NO: 21) (isolated, purified or produced through recombinant technologies). In one embodiment, the modulator comprises, or consists/consists essentially of, a synthetic fragment of SEQ ID Nos 22-24 (isolated, purified or produced through recombinant technologies). In one embodiment, the synthetic fragment comprises RBLs4 (SEQ ID NO: 22) (isolated, purified or produced through recombinant technologies). In one embodiment, the synthetic fragment comprises RBLs5 (SEQ ID NO: 23) (isolated, purified or produced through recombinant technologies). In one embodiment, the synthetic fragment comprises RBLs6 (SEQ ID NO: 24) (isolated, purified or produced through recombinant technologies).

The disclosure also provides methods to transiently increase vesicular transcytosis across human BBB endothelium in a subject in need thereof. In one embodiment, the subject is not pregnant. In one embodiment, vesicular transcytosis is increased by administration of an effective dose of one or more of the polypeptides of the disclosure, which are modulators of MFSD2A activity or function. In one embodiment, the modulator comprises, or consists/consists essentially of a fragment of SYNC2 (SEQ ID NO:5) (isolated, purified or produced through recombinant technologies) or of SEQ ID NOs: 3, 4, or 6 (isolated, purified or produced through recombinant technologies). In one embodiment, the modulator comprises, or consists/consists essentially of, a soluble fragment of SEQ ID Nos 3-24 or other syncytin (isolated, purified or produced through recombinant technologies). In one embodiment, the soluble fragment comprises SYNC2_{SU-CO} (SEQ ID NO. 7) (isolated, purified or produced through recombinant technologies). In one embodiment, the soluble fragment comprises SYNC2_{SU-CO-2} (SEQ ID NO. 8) (isolated, purified or produced through recombinant technologies). In one embodiment, the soluble fragment comprises SYNC2_{SU-CO-3} (SEQ ID NO: 9) (isolated, purified or produced through recombinant technologies). In one embodiment, the soluble fragment comprises SYNC2_{SU-CO} (C43) (SEQ ID NO: 10) (isolated, purified or produced through recombinant technologies). In one embodiment, the soluble fragment comprises SYNC2_{SU-CO-2 (C43)} (SEQ ID NO: 11) (isolated, purified or produced through recombinant technologies). In one embodiment, the soluble fragment comprises SYNC2_{SU-CO-3 (C43)} (SEQ ID NO: 12) (isolated, purified or produced through recombinant technologies). In one embodiment, the soluble fragment comprises SYNC2_{SU}-WT (SEQ ID NO: 13) (isolated, purified or produced through recombinant technologies). In one embodiment, the soluble fragment comprises SYNC2_{SU-WT-2} (SEQ ID NO. 14) (isolated, purified or produced through recombinant technologies). In one embodiment, the soluble fragment comprises SYNC2_{SU-WT-3} (SEQ ID NO: 15) (isolated, purified or produced through recombinant technologies). In one embodiment, the soluble fragment comprises SYNC2_{SU-C43S} (SEQ ID NO: 16) (isolated, purified or produced through recombinant technologies). In one embodiment, the soluble fragment comprises SYNC2_{SU-C43S-2} (SEQ ID NO: 17) (isolated, purified or produced through recombinant technologies). In one embodiment, the soluble fragment comprises SYNC2_{SU-C43S-3} (SEQ ID NO: 18) (isolated, purified or produced through recombinant technologies). In one embodiment, the soluble fragment comprises (SYNC2) RBL1 (SEQ ID NO: 19) (isolated, purified or produced through recombinant technologies). In one embodiment, the soluble fragment comprises (SYNC2) RBL2 (SEQ ID NO: 20) (isolated, purified or produced through recombinant technologies). In one embodiment, the soluble fragment comprises (SYNC2) RBL3 (SEQ ID NO: 21) (isolated, purified or produced through recombinant technologies). In one embodiment, the modulator comprises, or consists/consists essentially of, a synthetic fragment of SEQ ID Nos 22-24 (isolated, purified or produced through recombinant technologies). In one embodiment, the synthetic fragment comprises RBLs4 (SEQ ID NO: 22) (isolated, purified or produced through recombinant technologies). In one embodiment, the synthetic fragment comprises RBLs5 (SEQ ID NO: 23) (isolated, purified or produced through recombinant technologies). In one embodiment, the synthetic fragment comprises RBLs6 (SEQ ID NO: 24) (isolated, purified or produced through recombinant technologies).

The disclosure also provides methods to transiently increase BBB permeability to deliver therapeutic small-compounds, macromolecules, nanomaterials, into the brain in a subject in need thereof. In one embodiment, the subject is not pregnant. In one embodiment, the BBB permeability is increased by administration of an effective dose of one or more of the polypeptides of the disclosure, which are modulators of MFSD2A activity or function. In one embodiment, the modulator comprises, or consists/consists essentially of a fragment of SYNC2 (SEQ ID NO:5) (isolated, purified or produced through recombinant technologies) or of SEQ ID NOs: 3, 4, or 6 (isolated, purified or produced through recombinant technologies). In one embodiment, the modulator comprises, or consists/consists essentially of, a soluble fragment of SEQ ID Nos 3-24 or other syncytin (isolated, purified or produced through recombinant technologies). In one embodiment, the soluble fragment comprises SYNC2_{SU-CO}(SEQ ID NO. 7) (isolated, purified or produced through recombinant technologies). In one embodiment, the soluble fragment comprises SYNC2_{SU-CO-2} (SEQ ID NO. 8) (isolated, purified or produced through recombinant technologies). In one embodiment, the soluble fragment comprises SYNC2_{SU-CO-3} (SEQ ID NO: 9) (isolated, purified or produced through recombinant technologies). In one embodiment, the soluble fragment comprises SYNC2_{SU-CO}(c43) (SEQ ID NO: 10) (isolated, purified or produced through recombinant technologies). In one embodiment, the soluble fragment comprises SYNC2_{SU-CO-2} (c43) (SEQ ID NO: 11) (isolated, purified or produced through recombinant technologies). In one embodiment, the soluble fragment comprises SYNC2_{SU-CO-3} (c43) (SEQ ID NO: 12) (isolated, purified or produced through recombinant technologies). In one embodiment, the soluble fragment comprises SYNC2_{SU-WT} (SEQ ID NO: 13) (isolated, purified or produced through recombinant technologies). In one embodiment, the soluble fragment comprises SYNC2_{SU-WT-2} (SEQ ID NO. 14) (isolated, purified or produced through recombinant technologies). In one embodiment, the soluble fragment comprises SYNC2_{SU-WT-3} (SEQ ID NO: 15) (isolated, purified or produced through recombinant technologies). In one embodiment, the soluble fragment comprises SYNC2_{SU-C43S}(SEQ ID NO: 16) (isolated, purified or produced through recombinant technologies). In one embodiment, the soluble fragment comprises SYNC2_{SU-C43S-2} (SEQ ID NO: 17) (isolated, purified or produced through recombinant technologies). In one embodiment, the soluble fragment comprises SYNC2_{SU-C43S-3} (SEQ ID NO: 18) (isolated, purified or produced through recombinant technologies). In one embodiment, the soluble fragment comprises (SYNC2) RBL1 (SEQ ID NO: 19) (isolated, purified or produced through recombinant technologies). In one embodiment, the soluble fragment comprises (SYNC2) RBL2 (SEQ ID NO: 20) (isolated, purified or produced through recombinant technologies). In one embodiment, the soluble fragment comprises (SYNC2) RBL3 (SEQ ID NO: 21) (isolated, purified or produced through recombinant technologies). In one embodiment, the modulator comprises, or consists/consists essentially of, a synthetic fragment of SEQ ID Nos 22-24 (isolated, purified or produced through recombinant technologies). In one embodiment, the synthetic fragment comprises RBLs4 (SEQ ID NO: 22) (isolated, purified or produced through recombinant technologies). In one embodiment, the synthetic fragment comprises RBLs5 (SEQ ID NO: 23) (isolated, purified or produced through recombinant technologies). In one embodiment, the synthetic fragment comprises RBLs6 (SEQ ID NO: 24) (isolated, purified or produced through recombinant technologies). In one embodiment, the macromolecules are selected from antibodies, DNA, RNA. In one embodiment, the nanomaterials are selected from liposomes and gene therapy. In one embodiment, the modulator comprises a conjugate of said polypeptides with a therapeutic. In one embodiment, transport across the BBB of drugs conjugated with the polypeptides of the disclosure may also be facilitated through receptor-mediated mechanisms involving MFSD2A.

The disclosure provides that the effective dose for each of the foresaid methods may be different. The dosage of the therapeutic formulation, e.g., pharmaceutical composition, may vary widely, depending upon the nature of the condition, the frequency of administration, the manner of administration, the clearance of the agent from the subject, and the like. In particular embodiments, the initial dose can be larger, followed by smaller maintenance doses. In one embodiment, the transient inhibition of MFSD2A transport function in the BBB, transient increase in vesicular transcytosis across human BBB endothelium, or transiently increase in BBB permeability to deliver therapeutic small-compounds, macromolecules (e.g. antibodies) or nanomaterials (e.g. liposomes, gene therapy) has a duration of 1-60 minutes. In one embodiment, the duration is from 1, 2, 3, 4, 5-15, 16-20, 21-25, 26-30, 31-35, 36-40, 41-45, 46-50, 51-55, 56-60 minutes, or any interval/fraction in between. In one embodiment, the duration is 1 hour, 2, 3, 4, 5, 6, 7, 8, 9, 10, 11, 12, 13, 14, 15, 16, 17, 18, 19, 20, 21, 22, 23, 24 hours. In one embodiment, the duration is 1 day, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, possibly with repeated administration. In one embodiment, the activity of the SYNC2 polypeptides of the disclosure may be terminated by the administration of MFSD2A or MFSD2A_{CO} polypeptides, which may be reconstituted in liposomes or membrane mimetics. In one embodiment, the activity is determined by the polypeptide's half-life.

Modulation of the BBB permeability may be desirable in a large number of diseases or disorders. In one embodiment, the disease or disorder is selected from brain cancer, schizophrenia, depression and other mood disorders, ADD/ADHD, epilepsy, multiple sclerosis, Alzheimer's Disease and other neurodegenerative diseases associated with tau and/or beta amyloid plaque formation. In one embodiment, the methods of the disclosure may be used to treat, or in combination with drugs that treat, any disease or disorder of the central nervous system.

The disclosure also provides a method to assay/screen for compounds with the capability to inhibit sodium-dependent uptake by MFSD2A, wherein cells expressing MFSD2A (e.g., human MFSD2A_{WT} or MFSD2A_{CO}) are incubated for a predetermined period of time with an MFSD2A substrate/substrate analog and the effect of the compounds on the sodium-dependent substrate uptake is measured in the absence or presence of various concentrations of compounds under screening. In one embodiment, the MFSD2A substrate analog is 12:0 Lyso NBD PC (Avanti polar lipids). In one embodiment, purified SYNC2_{SU-CO} or other SYNC2 fragments identified herein as inhibitors of this transport can be used as positive controls.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

### EXAMPLES

### Example 1: Consensus Designs Enables Cryo-EM Structure Determination

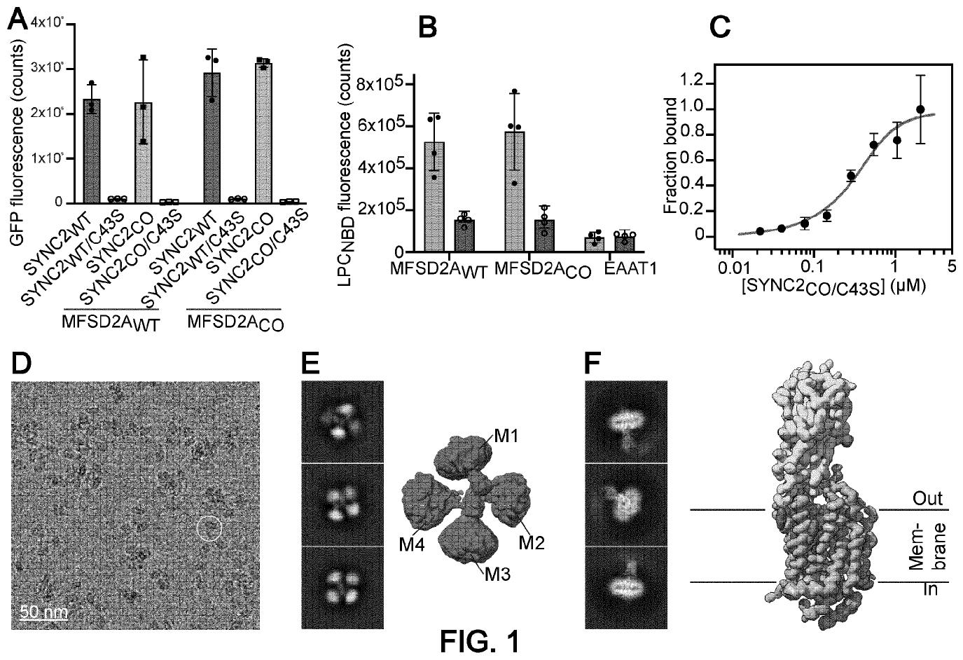

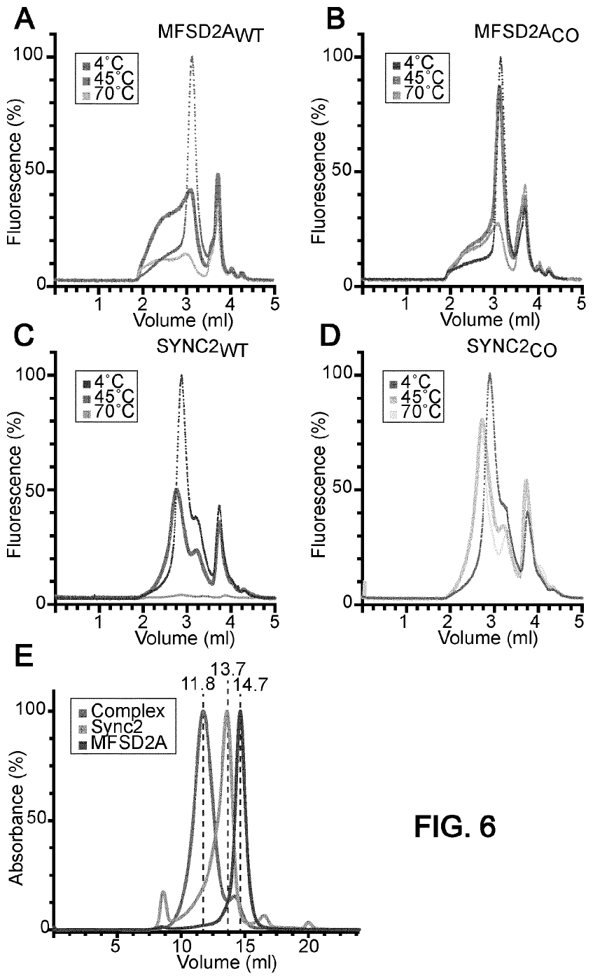

To obtain stable protein samples for in vitro complexation and structural analysis, the inventors exchanged amino acids in the sequence of wild type syncytin 2 (SYNC2_{WT}) and MFSD2A_{WT} for consensus residues of representative simian and vertebrate orthologs, respectively. Consensus designs, herein referred to as MFSD2A_{CO} and SYNC2_{CO}, were significantly more stable than WT counterparts in non-denaturing detergent solutions (FIG. 6A-D), and could be purified in milligram amounts with high-degree of homogeneity (FIG. 6E). Importantly, MFSD2A_{CO} and SYNC2_{CO} showed intact functional properties. First, the inventors probed cell-cell fusion using a split-GFP complementation assay (Cabantous et al., 2005), in which green-fluorescence reflects fusion of cells expressing different split-GFP components (FIG. 1A). Cultures of cells co-expressing the GFP parts with SYNC2_{WT}(SYNC2_{WT}/GFP₁₋₁₀) and MFSD2A_{WT} (MFSD2A_{WT}/GFP₁₁), respectively, yielded robust GFP-fluorescence signals, while negative control mutant (SYNC2_{WT-C43S}) that precludes SYNC2_{SU-CO} intra-subunit disulfide formation and cell fusion (Chen et al., 2008) showed negligible fluorescence levels. Indeed, cells co-expressing GFP parts with consensus-designs SYNC2_{CO}/GFP₁₋₁₀ and MFSD2A_{CO}/GFP₁₁, respectively, (or any combination of consensus and WT constructs) revealed high-fluorescence signals, demonstrating that the fusogenic mechanism of MFSD2A_{CO} and SYNC2_{CO} is intact. The inventors then probed MFSD2A sodium-dependent transport function using an LPC fluorescent analog (12:0 lyso NBD phosphocholine, referred to as LPCNBD) as substrate (FIG. 1B). MFSD2A_{CO} showed robust Na⁺-dependent uptake of LPCNBD (3.7±0.9-fold increase sodium- over choline-based condition), similar to MFSD2A_{WT} (3.4±0.5), while control cells expressing unrelated Na⁺-dependent neurotransmitter transporter EAAT1 lacked LPCNBD uptake (0.9±0.2). Overall, these functional experiments demonstrated that MFSD2A transport and receptor mechanisms are conserved.

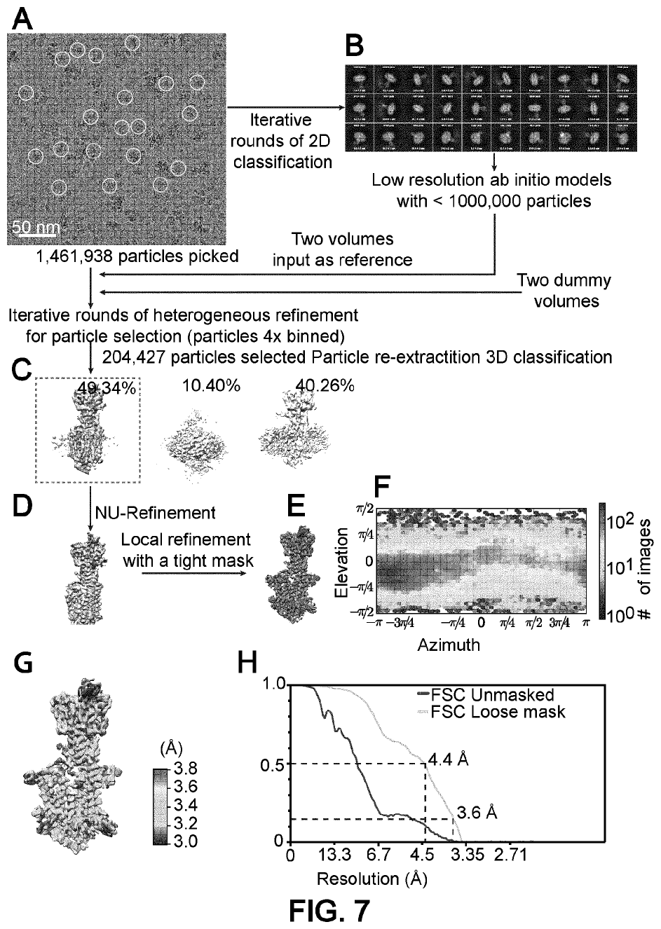

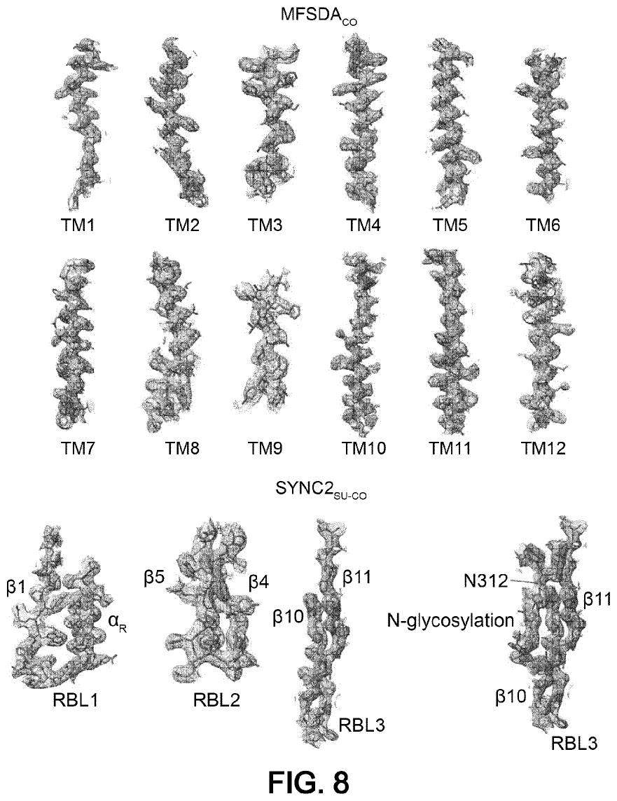

For cryo-EM structural determination of MFSD2A_{CO}-SYNC2_{CO} complex, the inventors used SYNC2_{CO}-C43S (SYNC2_{CO-C43S}) mutant to avoid detachment of surface and transmembrane subunits upon binding to MFSD2A_{CO}. SYNC2_{CO-C43S} binds MFSD2A_{CO} in non-denaturing detergent solutions with an apparent dissociation constant (K_{D}) of ˜100 nM, yielding stable complexes for cryo-EM analysis (FIG. 1C; FIG. 6E). Initial 2D classes and 3D reconstructions showed MFSD2A_{CO}-SYNC2_{CO-C43S} complexes with four transmembrane components enclosed in separate detergent micelles, consistent with three MFSD2A_{CO} molecules bound to trimeric SYNC2_{CO-C43S} (FIG. 1D,E). However, high flexibility within the complex precluded high-resolution 3D reconstructions. To overcome this problem, the inventors used a smaller box size in order to extract individual components, effectively increasing particle number and sample homogeneity, as it was recently shown in cryo-EM structure determination of human CD20 bound to divalent Fab fragments (Kumar et al., 2020). This approach enabled particle averaging over the three MFSD2A_{CO} molecules in the complex, yielding a cryo-EM map at an overall 3.6 Å resolution of SYNC2_{SU-CO} bound to MFSD2A_{CO} (FIG. 1F; Table 1; FIG. 7, 8). SYNC2_{TM}, the C-terminal transmembrane subunit of SYNC2 protomer, was not packed against SYNC2_{SU-CO}, indicating that the transmembrane subunit had undergone post-fusion transition, and that the two subunits remained linked through the inter-subunit disulfide bond, as well as a long flexible linker. Trimeric SYNC2_{TM} in the post-fusion state was not resolved in 3D cryo-EM reconstructions, likely due to lower number of particles, as well as alignment problems of micelles containing only three flexible transmembrane helices.

### Example 2: Structure of MFSD2A_{CO} in Outward-Facing and Partially-Occluded State

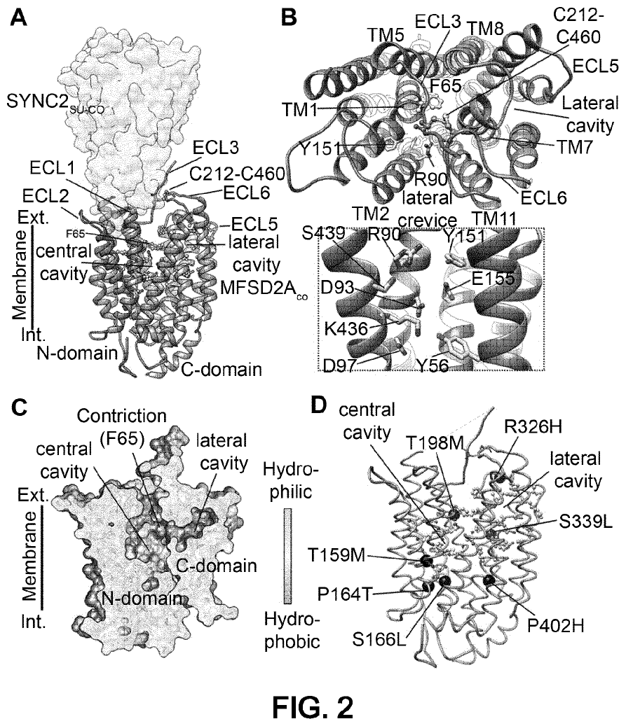

The structure of MFSD2A_{CO}-SYNC2_{SU-CO} complex was solved in the absence of LPC substrate, and MFSD2A_{CO} adopts an elusive apo outward-facing state of the transport cycle. MFSD2A_{CO} structure shows an amphiphilic central cavity between N- and C-domains (˜4,350 Å3) and strikingly, a large lateral hydrophobic cavity excavated on the C-domain (˜1,150 Å3) (FIG. 2A-C). The central cavity partly opens to the extracellular solution, as well as laterally towards the membrane through a narrow crevice between transmembrane-helix 2 (TM2) and TM11. Close proximity of TM1 and TM7 occludes this cavity from the extracellular solution, and extracellular loop 3 (ECL3), connecting TM5 and TM6, localizes atop the cavity making contacts with the C-domain, and linked through a disulfide bond to ECL6 (C212-C460).

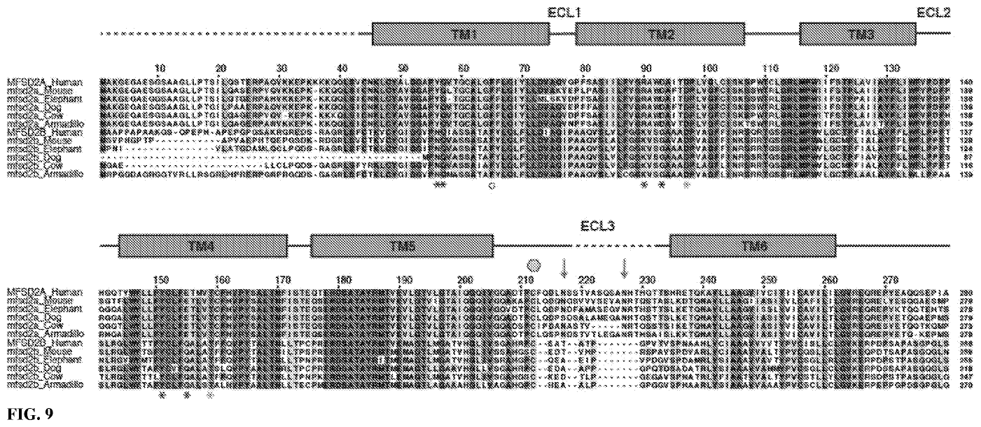

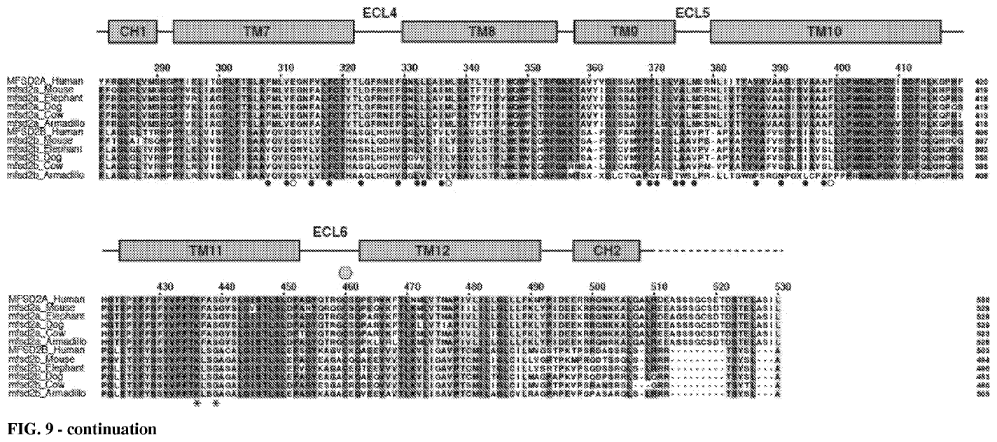

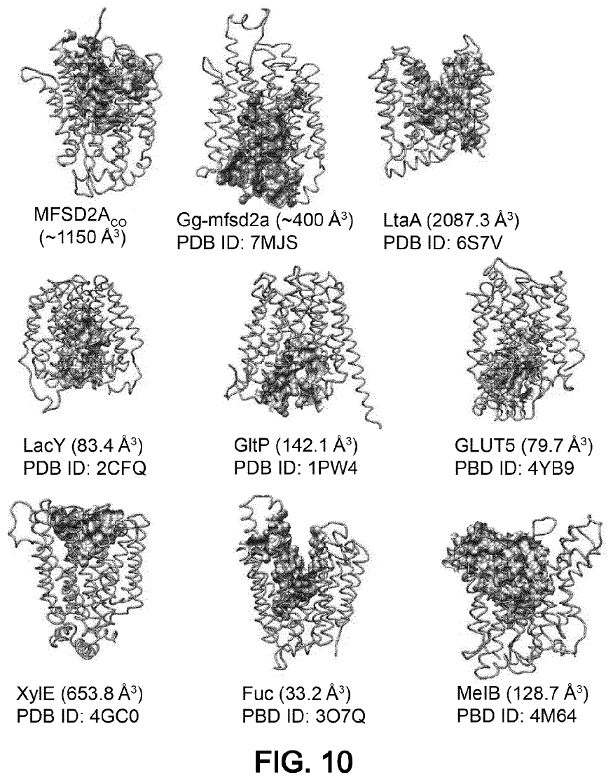

A cluster of highly-conserved polar residues face the bottom of the central cavity near halfway across the membrane (FIG. 2A,B; FIG. 9) (including Y56, Q57, R90, D93, D97, Y151, E155, T159, S439, and K436), and most of them are reported residues important for Na+-induced LPC transport (Cater et al., 2021; Ethayathulla et al., 2014; Quek et al., 2016). The hydrophobic lateral cavity is also lined by conserved residues (FIG. 2A,C; FIG. 9), and directly connects to the polar-residue cluster through a constriction formed by residues F65, E312, M337, and F399. The unique spatial arrangement of the polar-residue cluster, constriction, and lateral cavity appears optimal to bind and occlude LPC within the transporter from the outside. D93/E155 and R90/K436 residue pairs are ideally positioned to provide counter-charges for trimethyl-ammonium and phosphate groups of LPC head, respectively, while other residues in close proximity likely complete LPC-headgroup coordination (Y56, Q57, Y151, and S439), and contribute to Na+binding (D97, T159). In turn, amino-acid conservation and hydrophobicity in both constriction and lateral-cavity seem optimal to occlude LPC fatty-acid tail into the C-domain. Several lines of evidence further support binding of LPC to the above-mentioned structural elements: reported mutagenesis of residues in the central cluster, lateral cavity, and constriction showed that they are important for transport (Cater et al., 2021; Ethayathulla et al., 2014; Quek et al., 2016); several transport-inactivating and disease-causing MFSD2A mutations in humans localize to the vicinity of central (T159M, P164T, S166L), and lateral cavities (S339L, P402H, T198M, R326H) (Alakbarzade et al., 2015; Guemez-Gamboa et al., 2015; Harel et al., 2018; Scala et al., 2020); Na+-dependent MFSD2 transporters that selectively carry sphingosine-1-phophate (Kobayashi et al., 2018; Vu et al., 2017), namely MFSD2B, lack the acidic residue pair equivalent to D93/E155 in MFSD2A, and have instead glycine and glutamine residues (FIG. 9), respectively, likely reflecting structural adaptations to accommodate sphingosine-1-phophate phosphate terminal group, instead of the trimethyl-ammonium one in LPC; the structure of prokaryotic MFS lipid transporter LtaA in open outward-facing state revealed a large hydrophobic cavity in the C-domain well-suited to accommodate the lipid part of the substrate, and MFS transporters carrying non-lipid and smaller substrates lack such cavity (Zhang et al., 2020) (FIG. 10).

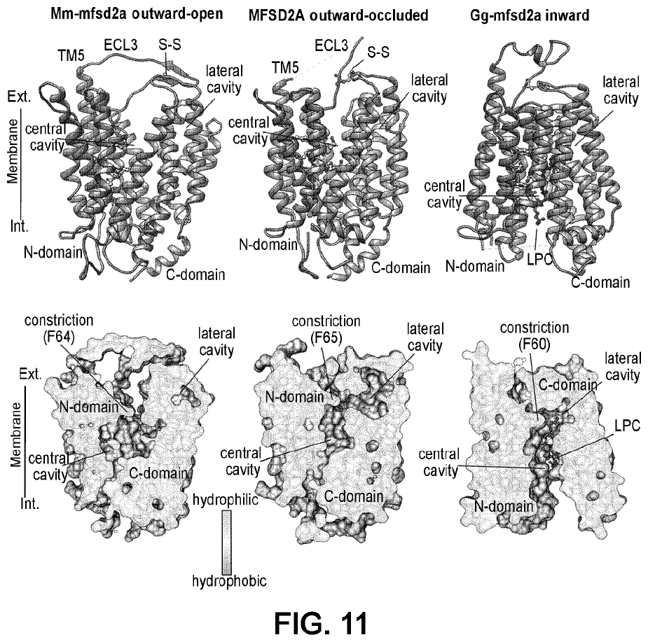

Structural comparison of MFSD2A_{CO} and Gg-mfsd2a inward-facing (Cater et al., 2021), as well as Mm-mfsd2a open outward-facing (Wood et al., 2021) states, respectively, revealed important conformational changes of the transport cycle (FIG. 11). Isomerization between outward- and inward-facing states occurs through rotations of N- and C-domains around the central polar-residue cluster alternately exposing central and lateral cavities to the extracellular and intracellular sides, resembling a classical MFS “rocker-switch” mechanism (Quistgaard et al., 2016; Yan, 2015). Notably, conformational changes in the extracellular half of the C-domain, including TM7-10, simultaneously contract the volume of the lateral cavity, and expand the constriction in the inward-facing state, suggesting that these changes contribute to release of the substrate on the cytoplasmic side. Consistently, Gg-mfsd2a cryo-EM map shows non-protein density for a bound LPC molecule with its acyl-chain “trapped” in the constriction, while the headgroup faces the cytoplasm, and is loosely wedged in a cytoplasmic crevice between TM5 and TM8. Moreover, in Mm-mfsd2a structure N- and C-domain separate further than in MFDS2A_{CO}, and the former lacks a hydrophobic cavity in the C-domain, further suggesting that occlusion of the central binding pocket is associated to opening of the hydrophobic cavity.

The above structural comparison showed that MFSD2A_{CO} adapted a classical MFS “rocker-switch” mechanism (Quistgaard et al., 2016; Yan, 2015) to simultaneously gate central and lateral cavities, and achieve translocation of both LPC headgroup and acyl-chain occluded within the transporter (discussed below). The presence of large C-domain hydrophobic cavities in both MFSD2A_{CO}, and LtaA (Zhang et al., 2020) further suggested that this mechanism is conserved across prokaryotic and human MFS lipid transport systems.

### Example 3: Structure of Endogenous Retroviral SYNC2_{SU-CO}

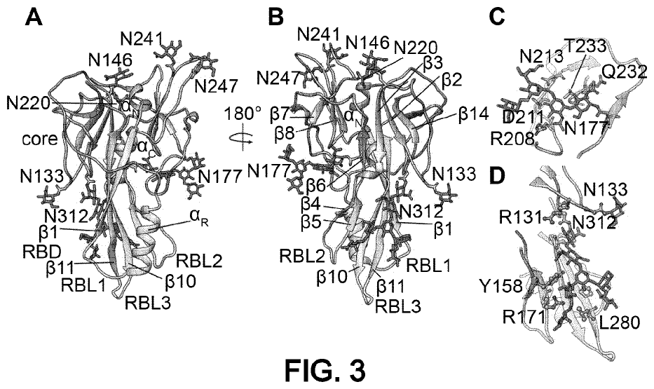

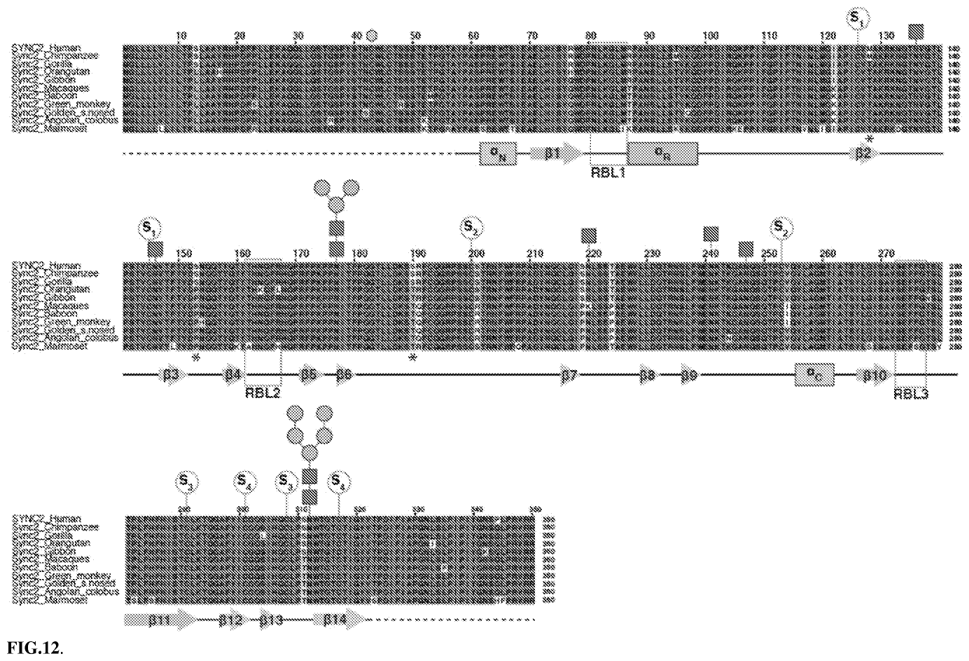

The structure of SYNC2_{SU-CO} represents a novel protein fold, and a search of the protein data bank (Holm, 2020) showed only marginal resemblance with envelopes from unrelated filoviruses (e.g. ebola virus, EBOV). SYNC2_{SU-CO} is arranged in two domains: a core, and a receptor binding domain (RBD) (FIG. 3A,B; FIG. 12). The core is a distorted beta-barrel that encloses a small central alpha-helix (αC), and it is capped on the opposite side of the RBD by N-terminal αN. Several loops pack around the distorted beta barrel and establish disulfide bonds with β2 (C126-C145), β11 (C291-C308), and β14 (C301-C317). Extensions from core structural elements form the RBD. This domain is a concave five-stranded anti-parallel β-sheet that partly enwraps αR. Notably, the three loops connecting β1-αR, β4-β5, and β10-β11 constitute the main contact region with MFSD2A, and the inventors refer to them as receptor-binding loops 1-3 (RBL1-3), respectively.

SYNC2_{SU-CO} displays extensive N-glycosylation on its surface, resembling to some extent the “glycan coat” observed in envelopes of exogenous and infectious human retroviruses (Watanabe et al., 2020): glycans at N146, N220, N241, and N247 in the core are distant from MFSD2A_{CO} interface and solvent exposed. In stark contrast, glycans at N177 and N312 are cradled in cavities on SYNC2_{SU-CO} surface, and remain more structured, displaying cryo-EM density that enabled modeling of high-mannose glycan molecules. N177 glycan binds a pocket delimited by loops between β6-β7 and β8-β9 in the core, and contributes to stabilize its fold through several contacts (FIG. 3C). In turn, N312 binds a shallow pocket in the RBD, and adopts a striking position in close proximity to RBL2 and RBL3 (FIG. 3D). Interestingly, SYNC2_{SU-CO} glycans concentrate on one side of its surface (FIG. 3B), suggesting that the side devoid of these molecules (FIG. 3A) provides the interface to interact with SYNC2_{TM} in the prefusion state.

### Example 4: SYNC2_{CO}-MFSD2A_{CO} Binding Interface

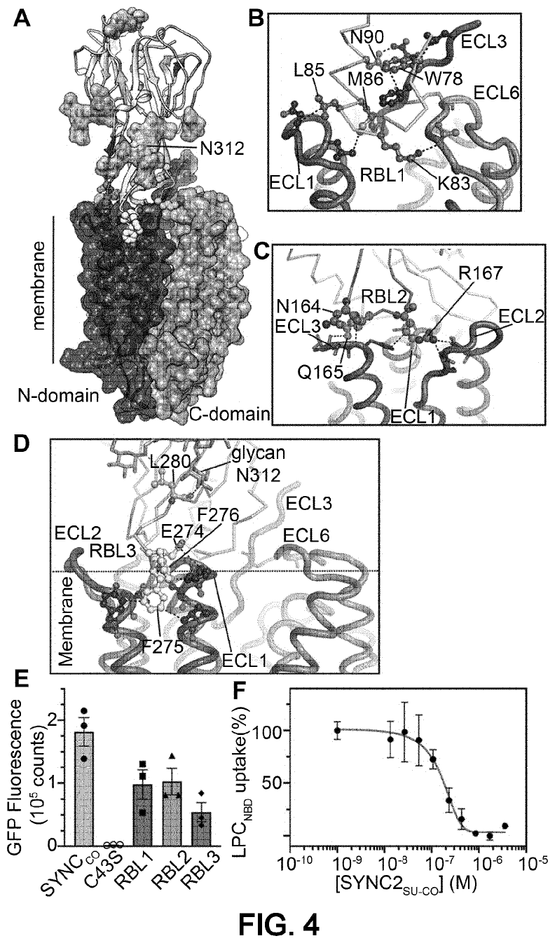

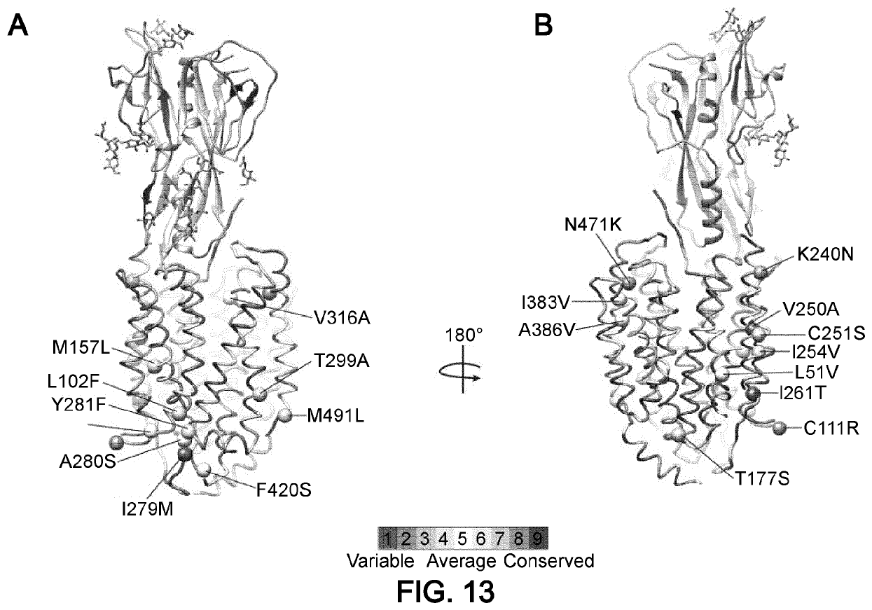

SYNC2_{SU-CO} binds MFSD2A_{CO} through an extensive network of interactions that buries a large and hydrophilic surface area of MFSD2A N-domain (˜1,200 Å2). The long axis of SYNC2_{SU-CO} is nearly parallel to that of the transporter and normal to the membrane plane, and the three RBLs of SYNC2_{CO} interact with poorly-conserved residues in shallow crevices on MFSD2A_{CO} surface (FIG. 4A; FIG. 13; Table 2)

RBL1 and αR in SYNC2_{CO} lean towards the central cavity of the transporter, and RBL1 occupies a crevice between ECL1 and ECL3, partly occluding the cavity through RBL1 residues K83-M86 (FIG. 4B). M86 is the only residue that contacts both ECL3 and ECL1, while K83 occupies the entrance of MFSD2A_{CO} central cavity, and constitutes a contact point of SYNC2_{CO} with the C-domain of MFSD2A_{CO}. RBL1 residue W78 forms an aromatic stack with F213 in ECL3, and the position of RBL1 is further stabilized through H-bonding of W78 and N90 with residues in ECL3, as well as interactions between L85-M86 and ECL1. RBL2 is the shortest loop in the RBD, and residues N164 and Q165 at its N-terminal end make contacts with the extracellular part of TM6. At the C-terminal end of RBL2, R167 sidechain inserts into a crevice between ECL1 and ECL2, stabilized by contacts with residues in those loops, as well as electrostatic interactions (FIG. 4C). Notably, longer RBL3 interacts more extensively with ECL1-2 crevice through residues V272-F276 (FIG. 4D). The sidechain of F275, and to a lesser extent that of F276, adopts a striking position tightly wedged in a pocket between TM2 and TM4, and fully inserted into the hydrophobic core of the micelle, in contact with non-protein density corresponding to detergent/lipid molecules. The position of RBL3 within the membrane plane suggests a direct role of membrane lipids in SYNC2_{CO} receptor binding. Consistent with the structural observation that all RBLs extensively engage in MFSD2A recognition, poly-alanine mutants in RBL1-3, respectively, targeting residues buried at SYNC2_{SU-CO}-MFSD2A_{CO} interface significantly decreased SYNC2 fusogenic function (FIG. 4E).

Another important feature of SYNC2_{SU-CO} binding interface is the close proximity of glycan N312 to RBL2 and RBL3. The position of this glycan with its arms wrapping around β10-β11 and establishing contacts with β4-β5 strongly suggests that N312-glycan stabilizes interactions between those loops and MFSD2A_{CO}. In agreement with this, SYNC2_{CO} glycosylation knock-out mutant N312Q, as well as N133Q that positions just atop, impaired cell-cell fusion activity (Cui et al., 2016).

Overall, these structural and functional results converge to show that SYNC2_{CO} establishes a complex network of protein-protein contacts with MFSD2A_{CO}, mainly on the surface of the N-terminal domain, that are further stabilized by protein-lipid, and protein-glycan interactions

### Example 5: Binding of SYNC2_{SU-CO} Inhibits MFSD2A_{CO} Transport Function

SYNC2_{CO}-binding mechanism precludes important conformational changes of MFSD2A_{CO} associated to LPC transport cycle. ECL3 in MFSD2A_{CO}, as well as RBL1 and αR in SYNC2_{SU-CO} partly occupy the space between N- and C-domains of the transporter on the extracellular side, preventing rotation of the domains and isomerization into the inward-facing state. Moreover, movements of TM1 and adjacent TMs within the N-domain are important during transitions between open and occluded states in MFS transporters (Quistgaard et al., 2016), and the extensive binding interface of SYNC2_{SU-CO} with ELC1 likely restricts those movements

Hence, the inventors hypothesized that SYNC2_{CO} binding impairs MFSD2A_{CO} dynamics and locks the transporter in a partially-occluded outward-facing state, inhibiting its transport function. To experimentally isolate the effect of SYNC2_{SU-CO} binding on MFSD2A_{CO} transport, from other effects related to SYNC2_{TM} post-fusion transition, the inventors engineered a soluble fragment encompassing the structure of SYNC2_{SU-CO}, and lacking SYNC2 membrane fusion machinery (SYNC2_{TM}). This strategy also enabled assaying MFSD2A sodium-dependent transport in cells in complete absence of detergents or membrane mimetic molecules to maintain transmembrane ionic gradients intact. Strikingly, purified SYNC2_{SU-CO} abolished Na+-dependent LPCNBD transport with a half-maximal inhibitory concentration (IC50) of ˜200 nM (FIG. 4F), comparable to the apparent K_{D} of SYNC2_{CO} observed in detergent solutions. These results demonstrate that SYNC2_{CO} binding inhibits MFSD2A transport function and therefore, that MFSD2A_{CO} receptor and transport molecular mechanisms are intertwined. Notably, they also show that purified and soluble SYNC2_{SU-CO} constitutes a potent inhibitor of MFSD2A-mediated LPC transport, and could be used to facilitate delivery of therapeutic molecules through the human BBB.

### Example 6: Methods

### Consensus Designs and Thermal Stability Assay

Consensus amino acids were calculated using JALVIEW (Waterhouse et al., 2009) from aligned sequences of representative vertebrate, and simian orthologs of MFSD2A and SYNC2, respectively, using Muscle (Edgar, 2004). Criteria to define consensus residues in alignments were as described before (Cirri et al., 2018). Consensus amino acid exchanges were simultaneously introduced into wild type sequences, and in the MFSD2A design care was taken to not introduce exchanges on the predicted extracellular surface of the transporter. As a result, MFSD2A consensus design shared more >96% sequence identity with WT, and encompasses exchanges at poorly-conserved positions mostly within the membrane plane (FIG. 13). Amino acid sequences of SYNC2 simian orthologs are highly conserved, and the final consensus design shares ˜99% (FIG. 12). Consistent with lack of substitutions at conserved positions in both MFSD2A and SYNC2 consensus designs, the constructs were fully functional.

### Protein Expression and Purification

Gene encoding human MFSD2A_{CO} was synthesized (GenScript) and subcloned into a pcDNA3.1(+) vector encompassing a N-terminal FLAG-tag, GFP, and PreScission protease cleavage site. Protein expression was done on HEK-293F (ATCC) by transient transfection, as described before (Canul-Tec et al., 2017) with small variations. Briefly, cells grown in FreeStyle™ 293 medium (ThermoScientific™) were transfected with linear 25K polyethyleneimine (PEI, Polysciences, Inc.) at a cell density of 2.5E6 cells/ml using 3 μg/ml of DNA. Valproic acid (VPA) was added to the culture at a final concentration of 2.2 mM 24 hours after transfection, and cells were grown for additional 24 hours before harvesting.

Cell pellets were resuspended and lysed in buffer containing: 50 mM HEPES pH 7.4, 200 mM NaCl, 5% v/v glycerol, and 1 mM EDTA, and supplemented with protease-inhibitor cocktail (1 mM PMFS, and protease-inhibitor cocktail from Sigma), 1% Dodecyl-β-D-Maltopyranoside (DDM, Anatrace), and 0.2% cholesteryl hemi-succinate tris salt (CHS, Anatrace), and incubated for one hour. Cell debris was removed by centrifugation in a benchtop centrifuge, and clear supernatants were obtained by ultracentrifugation. Solubilized MFSD2A_{CO} was purified by affinity chromatography using the anti-FLAG M2 affinity gel (Sigma) packed in a gravity column. Resin was pre-equilibrated in buffer A containing: 50 mM HEPES pH 7.4, 200 mM NaCl, 5% v/v glycerol, 0.03% DDM, and incubated with the transporter for one hour under rotation. Resin was extensively washed with buffer A and then the buffer was exchanged to buffer B containing: 50 mM HEPES pH 7.4, 200 mM NaCl, 5% v/v glycerol, 0.1% glyco-diosgenin (GDN, Anatrace). The protein was eluted in buffer containing: 50 mM HEPES pH 7.4, 200 mM NaCl, 5% v/v glycerol, 0.0084% GDN, and 100 μg/ml FLAG-peptide (Sigma); and digested with PreScission protease for two hours. Cleaved protein was concentrated to several mg/ml using 100 kDa MWCO concentrator (Corning® Spin-X® UF concentrators) and injected in a Superose 6 column (GE Healthcare Life Sciences) using a SEC buffer containing: 20 mM HEPES pH 7.4, 150 mM NaCl, 5% v/v glycerol, 0.0084% GDN. Purified MFSD2A_{CO} was immediately used, or flash frozen and stored at −80° C. All purification steps were done at 4° C.

SYNC2_{CO-C43S} expression was done using a similar protocol with small modifications. SYNC2_{CO-C43S} was synthesized (GenScript) and subcloned into a pcDNA3.1(+) vector encompassing C-terminal PreScission protease cleavage site, GFP, and Strep-tag. Protein purification was done as for MFSD2A_{CO} but using StrepTactin™ resin (GE Healthcare), and elution buffer supplemented with 2.5 mM d-Desthiobiotin (Sigma). Purified SYNC2_{CO-C43S} was kept at 4° C. and used immediately after purification.

For in vitro complexation of MFSD2A_{CO} and SYNC2_{CO-C43S}, purified protein samples were mixed and incubated for >1 hr at 4° C., using three-fold molar excess of SYNC2_{CO-C43S} over MFSD2A_{CO}. Excess SYNC2_{CO-C43S} was removed by SEC.

SYNC2_{SU-CO} was cloned in pcDNA3.1(+) vector with C-terminal EPEA-tag, and expressed as secreted protein in HEK-293F. Four days after transfection, culture supernatant was incubated with anti-EPEA tag resin (CaptureSelect™ C-tagXI Affinity Matrix, Thermo scientific) for 1 hour at 4° C., under rotation. Resin was then transferred to a gravity column, and extensively washed with buffer A (50 mM HEPES pH 7.4, 200 mM NaCl, 10% v/v glycerol). Protein was eluted with buffer containing 20 mM HEPES pH 7.4, 150 mM NaCl, 5% v/v glycerol 2 mM S-E-P-E-A peptide, concentrated using 30 kDa MWCO, and injected into Superose 6 SEC column equilibrated with buffer containing 20 mM HEPES pH 7.4, 150 mM NaCl, 5% v/v glycerol. Purified samples were immediately used, or flash frozen and stored at −80° C.

### Thermal Stability Assay

To assay the effect of consensus mutations on thermal-stability, pellets from small-scale cultures (4-8 ml) of transfected cells were resuspended in 800 μl of lysis buffer, dounce homogenised, and lysed adding by supplementing the buffer with 1% DDM and 0.2% CHS. Samples were under rotation for 1 hr and clear lysates were obtained by ultracentrifugation at 87,000 g for 40 min at 4° C. Clear lysates were heated at different temperatures for 20 min using a PCR thermocycler and ultracentrifuged again (87,000 g, 50 min) to remove precipitates. Samples were assayed in Sepax HPLC column equilibrated with 20 mM HEPES pH 7.4, 150 mM NaCl, 5% glycerol, 0.03% DDM. The output of the column was connected to a fluorimeter (Quanta Master, HORIBA) to quantify elution profiles.

### Cell Fusion Assays

The cell fusion assay was based on split-GFP complementation system (Cabantous et al., 2005). GFP β-strand 11 (GFP11) was fused to the C-terminus of MFSD2A using a previously reported strategy (Kaddoum et al., 2010), while a gene encoding GFP β3-strands 1-10 (GFP1-10) was cloned in pcDNA3.1(+) vector.

HEK-293T.17 (ATCC) adherent cells were grown in DMEM medium (Gibco) supplemented with 10% heat-inactivated fetal bovine serum (FBS, GE Healthcare Life Sciences), Penicillin/Streptomycin antibiotics (100 units/ml Penicillin and 100 μg/ml Streptomycin, Gibco), glutamine (2 mM), and minimum essential medium non-essential amino acids (1×MEM NEAA, Gibco). Cells cultured in 24-well plates were transfected with MFSD2A-GFP11 plasmid, or co-transfected with SYNC2 (WT or mutants) plus GFP1-10 plasmids, respectively, using Lipofectamine 2000 (Invitrogen), and following manufacturer recommendations. 24 hours after transfections, cells were gently washed with PBS, and those expressing SYNC2/GFP1-10 (or mutants) were re-suspended and layered over attached cells expressing MFSD2A-GFP11, and incubated at 37° C. for nearly three hours. Then, cells were detached, transferred to Eppendorf tubes, washed with PBS by centrifugation, and finally transferred to a 96-well plate for fluorescence quantification. GFP fluorescence was measured using CLARIOstar-Plus plate reader (BMG Labtech; λexcitation 470 nm and λemission 514 nm). Three biological experiments were quantified in triplicate samples.

### LPC-Analog Transport Assay

Sodium-dependent substrate uptake was measured in HEK-293T.17 grown and transfected as described above. 24 hours after transfection, cells were washed with pre-warmed PBS, and ˜1 million cells were pelleted and resuspended in 500 μl of transport buffer (20 mM HEPES pH 7.4, 150 mM NaCl, 1.7 mM KCl, 1.2 mM MgCl2, 2.5 mM CaCl2), 5 mM Glc), or control buffer (in which NaCl was substituted by choline-Cl). MFSD2A substrate analog 12:0 Lyso NBD PC (Avanti polar lipids) was added to a final concentration of 20 μM from 5 mM DMSO stock, and cells were incubated for 10 minutes at 37° C. Ligand excess was removed by centrifugation, and cells were resuspended in 200 μl of PBS and transferred to black 96-well flat-bottom plates (grenier). Fluorescence quantification was done in a micro-plate reader (CLARIOstar-Plus) using λexcitation 464 nm and λemission 531 nm. Three biological experiments were quantified in triplicate samples.

To probe the effect of SYNC2_{SU-CO} on MFSD2A function, cells were incubated with purified SYNC2_{SU-CO} at different concentrations for 10 minutes at 37° C., before assaying transport. Titration of transport inhibition was fitted to Hill-like equation using Prism (GraphPad).

### SYNC2 Binding Assay in Detergent Solutions

SYNC2 binding to MFSD2A was assayed in clear lysates of detergent-solubilized HEK-293F cells expressing mCherry (C-terminal) and GFP (N-terminal) fusion constructs of SYNC2 and MFSD2A, respectively, that enable protein quantification. Clear cells lysates were obtained by 1-hour solubilization in lysis buffer containing 50 mM HEPES pH 7.4, 200 mM NaCl, 5% v/v glycerol, 1 mM EDTA, protease-inhibitor cocktail, 1% DDM, and 0.2% CHS, followed by centrifugation (3,800g, 20 min). SYNC2-mCherry and GFP-MFSD2A protein concentrations in lysates were determined using purified standards in a micro-plate reader (CLARIOstar-Plus) set at 570/620 and 470/514 (λexcitation/λemission), respectively. MFSD2A-GFP lysate at 500 nM was incubated with 2 ml anti-FLAG M2 resin for 1 hour, fractionated in 8 equal parts placed on gravity columns and washed with 1 ml of wash buffer (50 mM HEPES pH 7.4, 200 mM NaCl, 5% (v/v) glycerol, 0.03% DDM, and 0.006% CHS). SYNC2-mCherry was added from a calibrated stock at 2 μM, and equilibrated for 1 hour. Unbound SYNC2-mCherry was removed with wash buffer, and samples were eluted with buffer containing: 20 mM HEPES pH 7.4, 150 mM NaCl, 5% v/v glycerol, 0.03% DDM, 0.006% CHS, and 100 μg/ml FLAG-peptide. All binding steps were done at 4° C. Eluted protein was transferred to a 96-well flat-bottom black plate for quantification of GFP and mCherry fluorescence. SYNC-mCherry fluorescence background was determined from samples with anti-FLAG resin that lack MFSD2A-GFP bound. SYNC2-mCherry subtracted fluorescence was fitted to a quadratic binding equation using Sigma Plot (Systat).

$$Y = ( \frac{( [ M ] + [ S ] + KD ) - \sqrt{{( - ( [ M ] + [ S ] + KD ) )}^{2} - 4 [ M ] [ S ]}}{2} ) / [ M ]$$

Where Y is the fraction of MFSD2A-GFP bound to SYNC2-mCherry, K_{D} is the apparent dissociation binding constant, and [M] and [S] are total MFSD2A-GFP, and SYNC2-mCherry concentrations, respectively.

### Electron Microscopy Sample Preparation and Data Acquisition

MFSD2A_{CO}-SYNC2_{CO-C43S} purified complex was applied to glow-discharged Au 200 mesh Quantifoil R1.2/1.3 holey carbon grids (Quantifoil), and vitrified using Vitrobot Mark IV (ThermoFisher). Typically, 4 μl of sample at 1.2-1.5 mg/ml were applied to the grids, and the Vitrobot chamber was maintained at 100% humidity and 4° C. Grids were screened in 200 keV Talos Arctica microscope (ThermoFisher) at the IECB cryo-EM imaging facility. Data collection was performed at 300 keV Titan Krios microscope (ThermoFisher) at EMBL-Heidelberg Cryo-Electron Microscopy Service Platform, equipped with K2 direct electron detector (Gatan). Images were recorded with SerialEM (Mastronarde, 2005) at 22,500×, corresponding to a pixel size of 0.814 Å. Dose rate was 3 electrons/pixel/s, and the defocus range was −0.5 to −1.8 μm. Images were collected for 8 s with 0.2 s subframes (total 40 subframes), corresponding to a total dose of 4π electrons/Å². In total, 7365 movies were recorded.

### Cryo-EM Data Processing, Model Building, and Structure Analysis

All data sets were processed with cryoSPARC v2 (Punjani et al., 2017). Movies were gain corrected, and aligned using in-built patch-motion correction routine. CTF parameters were estimated using in-built patch-CTF routine in cryoSPARC. Low-quality images were discarded automatically by setting a CTF-resolution threshold of 20 Å, as well as manually upon visual inspection. ˜2000 particles manually picked were used to generated 2D templates for autopicking, and this process was iterated several times. 1,461,938 auto-picked particles were 4× Fourier cropped and initially subjected to rounds of 2D classification, followed by ab initio classification. Ab initio volumes displaying secondary structural elements of MFSD2A_{CO} and SYNC2_{CO} were then used to re-classify the entire set of auto-picked particles (4× Fourier cropped) using rounds of heterogenous refinement. The inventors also included ab initio volumes without molecular features to generate “bad” classes. 199,300 selected particles were re-extracted without Fourier cropping and used for ab initio reconstruction and non-uniform refinement (Punjani et al., 2020) that yielded a ˜4 Å map. Further focused refinement masking out the detergent micelle yielded a final map at an overall 3.6 Å resolution, based on the “gold-standard” 0.143 FSC cut-off that was used for model building (EMDB-12935). In order to improve density corresponding to glycans and some surface loops, a second map with a slightly softer mask was calculated (overall 3.8 Å resolution), and was used to aid model building of such regions (EMDB-12934). The refined atomic models built with the two maps are identical within experimental error. Maps were visualized and analyzed using UCSF Chimera (Pettersen et al., 2004) and sharpened with phenix.autosharpen in PHENIX (Terwilliger et al., 2018).

MFSD2A_{CO}-SYNC2_{SU-CO} structure was built de novo using Coot (Emsley et al., 2010). Secondary-structure predictions using JPred (Drozdetskiy et al., 2015) and XtalPred-RF (Slabinski et al., 2007) were used to help initial sequence assignment. Atomic coordinates were refined using PHENIX (Adams et al., 2010). Structural models from the above-mentioned maps were identical within experimental error.

Structural analyses were carried out as follows: protein cavity calculations with CASTp 3.0 (Tian et al., 2018), protein-protein interfaces with PISA (Krissinel and Henrick, 2007), structural alignments and structural homology PDB search with DALI (Holm, 2020), and amino acid conservation surface mapping with ConSurf (Glaser et al., 2003).

### Example 7: Tables

TABLE 1
Cryo-EM data collection and processing

| Sample | MFSD2A_{CO}-SYNC2_{SU-CO} |
|---|---|
| Microscope | Titan Krios |
| Voltage (kV) | 300 |
| Camera | Gatan K2-Summit |
| Electron dose (e⁻/Å²) | 47.1 |
| Pixel size (Å) | 0.814 |
| Defocus range (μm) | −0.5 to −1.8 |
| Micrographs collected | 7365 |
| Symmetry imposed | none |
| Final Particle set (No.) | 94,703 |
| Map Resolution (Å) | 3.6 |
| FSC threshold | 0.143 |
| Sharpening B-factor | 61.8 |

Refinement

| | |
|---|---|
| Model resolution (Å) | 3.6 |
| FSC threshold | 0.143 |
| Protein residues | 716 |
| Non-hydrogen atoms | 5869 |
| Number of ligands (total) | 17 |
| NAG | 9 |
| BMA | 2 |
| MAN | 6 |
| RMSD bonds (Å) | 0.008 |
| RMSD angles (°) | 1.244 |
| Ramachandran favored | 96.34 |
| Ramachandran allowed | 3.66 |
| Ramachandran outlier | 0 |
| Rotamer outlier | 0.81 |
| All atom clashscore | 5.47 |
| Mol Probity score | 1.54 |

TABLE 2
Interactions between SYCN2 RBLs and MFSD2A_{CO}
Hydrogen bonds between SYNC2RBL1 and MFSD2A

| SYNC2_{RBL1} atom | MFSD2A_{CO} atom | Donor-acceptor atoms distance (Å) |
|---|---|---|
| W78_N^{ε1} | Q214_O | 3.25 |
| P80_O | T457_O^{γ1} | 3.67 |
| K83_N^{ζ} | Q456_O | 2.72 |
| M86_S^{δ} | R458_N^{H1} | 3.49 |
| R87_N | D73_O^{δ1} | 3.83 |
| N90_N^{δ2} | C212_O | 3.45 |
| N90_O^{δ1} | Q214_N^{ε2} | 2.89 |
| K97_N^{ζ} | Q214_O^{ε1} | 3.89 |

Van der Waals contacts between SYNC2RBL1 and MFSD2A_{CO}

| SYNC2_{RBL1} residues | MFSD2A_{CO} residues |
|---|---|
| W78 | L216 |
| L82 | F213 |
| G84 | L72 |
| L85 | D73, Q76 |
| M86 | L72, D73, P211, F213 |
| P88 | F166 |
| L92 | Y161, H163, P170 |
| L93 | Q214 |

Hydrogen bonds between SYNC2RBL2 and MFSD2A_{CO}

| SYNC2_{RBL2} atom | MFSD2A_{CO} atom | Donor-acceptor atoms distance (Å) |
|---|---|---|
| N164_N^{δ2} | T233_O | 3.74 |
| R167_N^{H1} | P137_O | 2.96 |
| R167_N^{H1} | Q143_N^{ε2} | 3.16 |

Van der Waals contacts between SYNC2RBL2 and MFSD2A_{CO}

| SYNC2_{RBL2} residues | MFSD2A_{CO} residues |
|---|---|
| N164 | S234 |
| Q165 | H235 |
| R167 | Q76, V136, H235 |

Hydrogen bonds between SYNC2RBL3 and MFSD2A_{CO}

| SYNC2_{RBL3} atom | MFSD2A_{CO} atom | Donor-acceptor atoms distance (Å) |
|---|---|---|
| S273_O^{γ} | Q76_O | 2.87 |
| F275_N | S81_O^{γ} | 2.69 |

Van der Waals contacts between SYNC2RBL3 and MFSD2A

| SYNC2_{RBL3} residues | MFSD2A_{CO} residues |
|---|---|
| V272 | V77 |
| S273 | Q143 |
| F275 | I84, T144, Y145 |

TABLE 3
Sequences Summary

| Sync2 - full length | | | SEQ ID NO: | |
|---|---|---|---|---|
| Name | C43 position | sequence | Amino acid | DNA |
| SYNC2_{WT} | WT | WT human | 3 | 25 |
| SYNC2_{WT-C43S} | C43S | | 4 | 26 |
| SYNC2_{CO} | WT | Thermostable | 5 | 27 |
| SYNC2_{CO-C43S} | C43S | consensus | 6 | 28 |

| Sync 2 fragments | | | | SEQ ID NO: | |
|---|---|---|---|---|---|
| Fragment | Name | C43 position | sequence | Amino acid | DNA |
| 1 | SYNC2_{SU-CO} | C43S | Thermostable | 7 | 29 |
| | SYNC2_{SU-CO (C43)} | WT | consensus | 10 | 32 |
| | SYNC2_{SU-WT} | WT | WT human | 13 | 35 |
| | SYNC2_{SU-C43S} | C43S | | 16 | 38 |
| 2 | SYNC2_{SU-CO-2} | C43S | Thermostable | 8 | 30 |
| | SYNC2_{SU-CO-2 (C43)} | WT | consensus | 11 | 33 |
| | SYNC2_{SU-WT-2} | WT | WT human | 14 | 36 |
| | SYNC2_{SU-C43S-2} | C43S | | 17 | 39 |
| 3 | SYNC2_{SU-CO-3} | C43S | Thermostable | 9 | 31 |
| | SYNC2_{SU-CO-3 (C43)} | WT | consensus | 12 | 34 |
| | SYNC2_{SU-WT-3} | WT | WT human | 15 | 37 |
| | SYNC2_{SU-C43S-3} | C43S | | 18 | 40 |
| | RBL1 | n.a. | WT human | 19 | |
| | RBL2 | n.a. | WT human | 20 | |
| | RBL3 | n.a. | WT human | 21 | |
| | RBLs4 | n.a. | synthetic | 22 | |
| | RBLs5 | n.a. | synthetic | 23 | |
| | RBLs6 | n.a. | synthetic | 24 | |

LISTING OF SEQUENCES

SEQ ID NO: 1
Wild type human MFSD2A (isoform 2). Referred to in the Examples as MFSD2A_{WT}
NCBI Reference Sequence: NP_116182.2
https://www.ncbi.nlm.nih.gov/protein/NP_116182.2?report=genbank&log$=prottop&blast_
rank=1&RID=VR2Y3ZNR01R
Uniprot ID: Q8NA29-2
>MFSD2A_{WT}
MAKGEGAESGSAAGLLPTSILQSTERPAQVKKEPKKKKQQLSVCNKLCYALGGAPYQVT
GCALGFFLQIYLLDVAQVGPFSASIILFVGRAWDAITDPLVGLCISKSPWTCLGRLMPWIIFS
TPLAVIAYFLIWFVPDFPHGQTYWYLLFYCLFETMVTCFHVPYSALTMFISTEQTERDSATA
YRMTVEVLGTVLGTAIQGQIVGQADTPCFQDLNSSTVASQSANHTHGTTSHRETQKAYLL
AAGVIVCIYIICAVILILGVREQREPYEAQQSEPIAYFRGLRLVMSHGPYIKLITGFLFTSLAFM
LVEGNFVLFCTYTLGFRNEFQNLLLAIMLSATLTIPIWQWFLTRFGKKTAVYVGISSAVPFLI
LVALMESNLIITYAVAVAAGISVAAAFLLPWSMLPDVIDDFHLKQPHFHGTEPIFFSFYVFFT
KFASGVSLGISTLSLDFAGYQTRGCSQPERVKFTLNMLVTMAPIVLILLGLLLFKMYPIDEER
RRQNKKALQALRDEASSSGCSETDSTELASIL
SEQ ID NO: 2
Thermostable consensus MFSD2A mutant. Referred to in the Examples as MFSD2A_{CO}
(MFSD2A consensus)
>MFSD2A_{CO}
MAKGEGAESGSAAGLLPTSILQSTERPAQVKKEPKKKKQQLSICNKLCYAVGGAPYQVTG
CALGFFLQIYLLDVAQVGPFSASIILFVGRAWDAITDPLVGFCISKSSWTRLGRLMPWIIFST
PLAVIAYFLIWFVPDFPHGQTYWYLLFYCLFETLVTCFHVPYSALTMFISTEQSERDSATAY
RMTVEVLGTVLGTAIQGQIVGQADTPCFQDLNSSTVASQSANHTHGTTSHRETQNAYLLA
AGVIASIYVICAVILTLGVREQREPYEAQQSEPMSFFRGLRLVMSHGPYIKLIAGFLFTSLAF
MLVEGNFALFCTYTLGFRNEFQNLLLAIMLSATLTIPIWQWFLTRFGKKTAVYVGISSAVPF
LILVALMESNLIVTYVVAVAAGISVAAAFLLPWSMLPDVIDDFHLKQPHSHGTEPIFFSFYVF
FTKFASGVSLGISTLSLDFAGYQTRGCSQPERVKFTLKMLVTMAPIVLILLGLLLFKLYPIDE
ERRRQNKKALQALRDEASSSGCSETDSTELASIL
SEQ ID NO: 3
Wild type human SYNCYTIN 2. Referred to in the Examples as SYNC2_{WT}
NCBI Reference Sequence: NP_997465.1
https://www.ncbi.nlm.nih.gov/protein/NP_997465.1?report=genbank&log$=prottop&blast_
rank=1&RID=VRAT57J0013
Uniprot ID: P60508
>SYNC2_{WT}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPLTGP
LVSLLLLLLFGPCLLNLITQFVSSRLQAIKLQTNLSAGRHPRNIQESPF
SEQ ID NO: 4
SYNC2 C43S mutant inhibiting cell-cell fusion. Refer to in the Examples as SYNC2_{WT}-C43S
>SYNC2_{WT-C43S}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPLTGP
LVSLLLLLLFGPCLLNLITQFVSSRLQAIKLQTNLSAGRHPRNIQESPF
SEQ ID NO: 5
Thermostable consensus SYNC2 mutant. Referred to in the Examples as SYNC2_{CO}
(SYNC2 consensus)
>SYNC2_{CO}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPFIGP
LVSLLLLLLFGPCLLNLITQFVSSRLQAIKLQTNLSAGRHPRNIQESPF
SEQ ID NO: 6
Thermostable consensus SYNC2 mutant with additional mutation C43S inhibiting
cell-cell fusion. Referred to in the Examples as SYNC2_{CO-C43S} (SYNC2 consensus)
>SYNC2_{CO}-C43S
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPFIGP
LVSLLLLLLFGPCLLNLITQFVSSRLQAIKLQTNLSAGRHPRNIQESPF
SEQ ID NO: 7
Thermostable consensus SYNC2 fragment mutant with additional mutation C43S
inhibiting cell-cell fusion. Referred to in the Examples as SYNC2_{SU-CO}
>SYNC2_{SU-CO}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAP
SEQ ID NO: 8
Thermostable consensus SYNC2 fragment 2 mutant with additional mutation C43S
inhibiting cell-cell fusion. This fragment is not included in the Examples, but it is more
stable than SYNC2_{SU-CO}, and it has the same effect MFSD2A activity, and therapeutic
applications.
>SYNC2_{SU-CO-2}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEG
SEQ ID NO: 9
Thermostable consensus SYNC2 fragment 3 mutant with additional mutation C43S
inhibiting cell-cell fusion..
>SYNC2_{SU-CO-3}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSP
SEQ ID NO: 10
Thermostable consensus SYNC2 fragment.
>SYNC2_{SU-CO(C43)}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAP
SEQ ID NO: 11
Thermostable consensus SYNC2 fragment 2.
>SYNC2_{SU-CO-2 C43)}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEG
SEQ ID NO: 12
Thermostable consensus SYNC2 fragment 3.
>SYNC2_{SU-CO-3(C43)}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSP
SEQ ID NO: 13
WT SYNC2 fragment 1
>SYNC2_{SU-WT}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAP
SEQ ID NO: 14
WT SYNC2 fragment 2
>SYNC2_{SU-WT-2}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEG
SEQ ID NO: 15
WT SYNC2 fragment 3
>SYNC2_{SU-WT-3}
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSP
SEQ ID NO: 16
SYNC2 C43S mutant fragment.
>SYNC2_{SU-C43S}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAP
SEQ ID NO: 17
SYNC2 C43S mutant fragment 2.
>SYNC2_{SU-C43S-2}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEG
SEQ ID NO: 18
SYNC2 C43S mutant fragment 3.
>SYNC2S_{U-C43S-3}
MGLLLLVLILTPSLAAYRHPDFPLLEKAQQLLQSTGSPYSTNSWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSP
SYNC2 peptides
SEQ ID NO: 19
>RBL1 (Residue 70 to 108)
AELHISYRWDPNLKGLMRPANSLLSTVKQDFPDIRQKPP
SEQ ID NO: 20
>RBL2 (Residue 157 to 175)
TYQTYTHNQFRHQPRFPKP
SEQ ID NO: 21
>RBL3 (Residue 264 to 294)
TSYLGISAVSEFFGTSLTPLFHFHISTCLKT
SEQ ID NO: 22
>RBLs4 (RBL 2 + 3 + 1)
TYQTYTHNQFRHQPRFPKPGNGTSYLGISAVSEFFGTSLTPLFHFHISTCLKTPGQGAELH
ISYRWDPNLKGLMRPANSLLSTVKQDFPDIRQKPP
SEQ ID NO: 23
>RBLs5 (RBL 2 + 3)
TYQTYTHNQFRHQPRFPKPGNGTSYLGISAVSEFFGTSLTPLFHFHISTCLKT
SEQ ID NO: 24
>RBLs6 (RBL 3 + 1)
TSYLGISAVSEFFGTSLTPLFHFHISTCLKTPGQGAELHISYRWDPNLKGLMRPANSLLSTV
KQDFPDIRQKPP
Codon optimized DNA sequences
SEQ ID NO: 25
Codon optimized DNA sequence coding for wild type human SYNCYTIN 2
>SYNC2_{WT} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggcacctggaagtggttctcctgggtgctgcccctgaccggaccactggtgtctctgctgctgctgctgctgttc
ggcccttgtctgctgaacctgatcacacagtttgtgagctccagactgcaggccatcaagctgcagaccaatctgagcgccggc
aggcacccaaggaacatccaggagtcccctttt
SEQ ID NO: 26
Codon optimized DNA sequence coding for SYNC2 C43S mutant inhibiting cell-cell
fusion
>SYNC_{2WT-C43S} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggcacctggaagtggttctcctgggtgctgcccctgaccggaccactggtgtctctgctgctgctgctgctgttc
ggcccttgtctgctgaacctgatcacacagtttgtgagctccagactgcaggccatcaagctgcagaccaatctgagcgccggc
aggcacccaaggaacatccaggagtcccctttt
SEQ ID NO: 27
Codon optimized DNA sequence coding for thermostable consensus SYNC2 mutant .
SYNC2_{CO} (SYNC2 consensus)
>SYNC2_{CO} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggcacctggaagtggttctcctgggtgctgcccttcatcggaccactggtgtctctgctgctgctgctgctgttcg
gcccttgtctgctgaacctgatcacacagtttgtgagctccagactgcaggccatcaagctgcagaccaatctgagcgccggca
ggcacccaaggaacatccaggagtcccctttt
SEQ ID NO: 28
Codon optimized DNA sequence coding for thermostable consensus SYNC2 mutant
with additional mutation C43S inhibiting cell-cell fusion SYNC2co-C43S (SYNC2
consensus)
>SYNC2_{CO-C43S} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggcacctggaagtggttctcctgggtgctgcccttcatcggaccactggtgtctctgctgctgctgctgctgttcg
gcccttgtctgctgaacctgatcacacagtttgtgagctccagactgcaggccatcaagctgcagaccaatctgagcgccggca
ggcacccaaggaacatccaggagtcccctttt
SEQ ID NO: 29
Codon optimized DNA sequence coding for thermostable consensus SYNC2
fragment mutant with additional mutation C43S inhibiting cell-cell fusion to in the
SYNC2_{SU-CO}
>SYNC2_{SU-CO} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgcccca
SEQ ID NO: 30
Codon optimized DNA sequence coding for thermostable consensus SYNC2
fragment 2 mutant with additional mutation C43S inhibiting cell-cell fusion SYNC2_{SU-CO-2}
>SYNC2_{SU-CO-2} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggc
SEQ ID NO: 31
Codon optimized DNA sequence coding for thermostable consensus SYNC2
fragment 3 mutant with additional mutation C43S inhibiting cell-cell fusion SYNC2_{SU-CO-3}
>SYNC2_{SU-CO-3} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactcccca
SEQ ID NO: 32
Codon optimized DNA sequence coding for thermostable consensus SYNC2
fragment SYNC2_{SU-CO(C43)}
>SYNC2_{SU-CO(C43)} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgcccca
SEQ ID NO: 33
Codon optimized DNA sequence coding for thermostable consensus SYNC2
fragment 2 SYNC2_{SU-CO-2(C43)}
>SYNC2_{SU-CO-2(C43)} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggc
SEQ ID NO: 34
Codon optimized DNA sequence coding for thermostable consensus SYNC2
fragment 3 SYNC2_{SU-CO-3(C43)}
>SYNC2_{SU-CO-3(C43)} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgaccgccaagagaaagaacggcacaaatgtgggca
ccctgcccagcaccgtgtgcaatgtgacctttacagtggaccctaaccagcagacctaccagacctatacacacaatcagttc
cggcaccagcccagatttcctaagccacccaacatcaccttcccccagggcacactgctggataagtctacaaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactcccca
SEQ ID NO: 35
Codon optimized DNA sequence coding for WT SYNC2 fragment 1 SYNC2_{SU-WT}
>SYNC2_{SU-WT} DNA
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgcccca
SEQ ID NO: 36
Codon optimized DNA sequence coding for WT SYNC2 fragment 2 SYNC2_{SU-WT-2}
>SYNC2_{SU-WT-2}
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggc
SEQ ID NO: 37
Codon optimized DNA sequence coding for WT SYNC2 fragment 3 SYNC2_{SU-WT-3}
>SYNC2_{SU-WT-3}
atgggcctgctgctgctggtgctgatcctgaccccaagcctggccgcatacaggcacccagacttccccctgctggagaaggc
ccagcagctgctgcagtctaccggcagcccttattccacaaactgctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactcccca
SEQ ID NO: 38
Codon optimized DNA sequence coding for SYNC2 C43S mutant fragment SYNC2_{SU-C43S}
>SYNC2_{SU-C43S} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgcccca
SEQ ID NO: 39
Codon optimized DNA sequence coding for SYNC2 C43S mutant fragment 2
SYNC2_{SU-C43S-2}
>SYNC2_{SU-C43S-2} DNA
atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactccccactgcctcgggtgcggagagcaatccactttatcccactgctggccggcctgggaat
cctggcaggaacaggaaccggaatcgcaggaatcaccaaggccagcctgacatatagccagctgtccaaggagatcgcc
aacaatatcgacacaatggccaaggccctgaccacaatgcaggagcagatcgattccctggccgcagtggtgctgcagaat
aggaggggcctggacatgctgaccgcagcacagggaggaatctgcctggccctggatgagaagtgctgtttctgggtgaacc
agagcggcaaggtgcaggataatatccggcagctgctgaaccaggcctcctctctgcgggagagagccacacagggctgg
ctgaattgggagggc
SEQ ID NO: 40
Codon optimized DNA sequence coding for SYNC2 C43S mutant fragment 3
SYNC2_{SU-C43S-3}
>SYNC2_{SU-C43S-3} DNA
Atgggcctgctgctgctggtgctgatcctgaccccaagcctggccgcatacaggcacccagacttccccctgctggagaaggc
ccagcagctgctgcagtctaccggcagcccttattccacaaacagctggctgtgcaccagctcctctaccgagacaccaggca
cagcctaccctgcctctccacgcgagtggaccagcatcgaggccgagctgcacatctcctatagatgggatccaaatctgaag
ggcctgatgcgccccgccaactctctgctgagcaccgtgaagcaggacttccctgatatccggcagaagccccctatcttcggc
ccaatctttacaaacatcaatctgatgggcatcgccccaatctgcgtgatggccaagagaaagaacggcacaaatgtgggca
ccctgcccagcaccgtgtgcaatgtgacctttacagtggacagcaaccagcagacctaccagacctatacacacaatcagttc
cggcaccagcccagatttcctaagccacccaacatcaccttcccccagggcacactgctggataagtctagcaggttttgcca
gggccgcccaagctcctgtagcacccggaatttctggtttagacccgccgactacaatcagtgcctgcagatctctaacctgtct
agcacagccgagtgggtgctgctggatcagaccaggaactccctgttctgggagaataagacaaagggcgccaaccagtct
cagaccccttgcgtgcaggtgctggccggaatgacaatcgccacctcctacctgggcatctccgccgtgtctgagttctttggca
catctctgacccccctgttccactttcacatcagcacctgcctgaagacacagggcgccttttatatctgcggccagtccatccac
cagtgtctgccttctaactggaccggcacatgtaccatcggctacgtgacccctgacatcttcatcgccccaggcaatctgagcc
tgcctatcccaatctatggcaactcccca
Sequences in FIG. 9:
SEQ ID NO: 41
Wild type human MFSD2A (isoform 2). Referred to in the figure as MFSD2A_Human
NCBI Reference Sequence: NP_116182.2
>MFSD2A_Human
MAKGEGAESGSAAGLLPTSILQSTERPAQVKKEPKKKKQQLSVCNKLCYALGGAPYQVT
GCALGFFLQIYLLDVAQVGPFSASIILFVGRAWDAITDPLVGLCISKSPWTCLGRLMPWIIFS
TPLAVIAYFLIWFVPDFPHGQTYWYLLFYCLFETMVTCFHVPYSALTMFISTEQTERDSATA
YRMTVEVLGTVLGTAIQGQIVGQADTPCFQDLNSSTVASQSANHTHGTTSHRETQKAYLL
AAGVIVCIYIICAVILILGVREQREPYEAQQSEPIAYFRGLRLVMSHGPYIKLITGFLFTSLAFM
LVEGNFVLFCTYTLGFRNEFQNLLLAIMLSATLTIPIWQWFLTRFGKKTAVYVGISSAVPFLI
LVALMESNLIITYAVAVAAGISVAAAFLLPWSMLPDVIDDFHLKQPHFHGTEPIFFSFYVFFT
KFASGVSLGISTLSLDFAGYQTRGCSQPERVKFTLNMLVTMAPIVLILLGLLLFKMYPIDEER
RRQNKKALQALRDEASSSGCSETDSTELASIL
SEQ ID NO: 42
mfsd2a Mouse
>MFSD2A_MUS/1-529 sodium-dependent lysophosphatidylcholine symporter 1 [Mus musculus]
>gi|81905913|sp|Q9DA75.1|NLS1_MOUSE RecName: Full = Sodium-dependent
lysophosphatidylcholine symporter 1; Short = NLS1; Short = Sodium-dependent LPC
symporter 1; AltName: Full = Major facilitator superfamily domain-containing protein 2A
[Mus musculus] >gi|12839017|dbj|BAB24407.1|unnamed protein product [Mus musculus]
>gi|38014379|gb|AAH60526.1|Major facilitator superfamily domain containing 2 [Mus
musculus] >gi|148698449|gb|EDL30396.1|major facilitator superfamily domain containing 2
[Mus musculus]
MAKGEGAESGSAAGLLPTSILQASERPVQVKKEPKKKQQLSICNKLCYAVGGAPYQLTGC
ALGFFLQIYLLDVAKVEPLPASIILFVGRAWDAFTDPLVGFCISKSSWTRLGRLMPWIIFSTP
LAIIAYFLIWFVPDFPSGTFLWYLLFYCLFETLVTCFHVPYSALTMFISTEQSERDSATAYRM
TVEVLGTVIGTAIQGQIVGQAKAPCLQDQNGSVVVSEVANRTQSTASLKDTQNAYLLAAGII
ASIYVLCAFILILGVREQRELYESQQAESMPFFQGLRLVMGHGPYVKLIAGFLFTSLAFMLV
EGNFALFCTYTLDFRNEFQNLLLAIMLSATFTIPIWQWFLTRFGKKTAVYIGISSAVPFLILVA
LMERNLIVTYVVAVAAGVSVAAAFLLPWSMLPDVIDDFHLKHPHSPGTEPIFFSFYVFFTKF
ASGVSLGVSTLSLDFANYQRQGCSQPEQVKFTLKMLVTMAPIILILLGLLLFKLYPIDEEKRR
QNKKALQALREEASSSGCSDTDSTELASIL
SEQ ID NO: 43
mfsd2a_Elefant
>MFSD2A_LOXODONTA/1-528 Major facilitator superfamily domain containing 2A
OS = Loxodonta africana OX = 9785 GN = MFSD2A PE = 4 SV = 1
MAKGEGAEGGSAAGLLPTSILQTGERPAHVKKEPKKKQQLSICNKLCYAVGGAPYQTGCA
LGFFLQIYLLMLSKVDPFSASIILFVGRAWDAITDPLVGFCISKSSWTRLGRLMPWIIFSTPP
AIIAYFLIWFVPDFPQGQALWYLLFYCLFETLVTCFHVPYSALTMFISTEQSERDSATAYRM
TVEVLGTVLGTAIQGQIVGQVDTPCLQDPNDFAMASEGVNRTHSTTSLKETQNAYLLAAG
VISSLYVICAVILTLGVREQREPYKTQQTEHTSFFRGLRLVMSHGPYVRLIAGFLFTSLAFML
VEGNFALFCTYTLGFRNEFQNLLLAVMLSATFTIPVWQWFLTRFGKKTAVYIGISSAVPFLIL
VALMDSNLIVTYVVAVAAGISVAAAFLLPWSMLPDVIDDFHLKQPHSHGTEPIFFSFYVFFT
KFASGVSLGISTLSLDFAGYQTRGCSQPARVKFTLKMLVTMAPIVLILLGLLLFKLYPIDEEK
RRQNKKALQALRDEAGSSGCSDTDSTELASIL
SEQ ID NO: 44
Mfsd2a_Dog
>MFSD2A_CANIS/1-529 PREDICTED: major facilitator superfamily domain-containing
protein 2A isoform X2 [Canis lupus familiaris]
MAKGEGAESGSAAGLLPTGILPAAERPAQVKKEPKKKQQLSICNKLCYAVGGAPYQVTGC
ALGFFLQIYLLDVAQVEPFFASIILFVGRAWDAFTDPLVGFCISKSSWTRLGRLMPWIIFSTP
LAIIAYFLIWFVPDFPRGQALWYLLFYCLFETLVTCFHVPYSALTMFISTEQSERDSATAYR
MTVEVLGTVLGTAIQGQIVGQADTPCLQDPSDSALAMEGANHTQSTTSLKETQNAYLLAA
GVIASIYVICAVILTLGVREQREPYETQQAEPMSFFRGLRLVMSHGPYVKLIAGFLFTSLAF
MLVEGNFALFCTYTLGFRNEFQNLLLAIMLSATFTIPIWQWFLTRFGKKTAVYVGISSAVPF
LILVAFMESNLIVTYVVAVAAGISVAAAFLLPWSMLPDVIDDFHLKQPQSHGTEPIFFSFYVF
FTKFASGVSLGISTLSLDFAGYQTRGCSQPARVKFTLKLLVTIAPIVLILLGLLLFKLYPIDEEK
RRQNKKALQALREEASSSGCSDTDSTELASIL
SEQ ID NO: 45
mfsd2a cow
>MFSD2A_BOS/1-523 PREDICTED: sodium-dependent lysophosphatidylcholine
symporter 1 isoform X2 [Bos taurus]
MAKGEGAESGSAAGLLPTGILQAGERPVQVKEPKKKKQLSICNKLCYAVGGAPYQVTGCA
LGFFLQIYLLDVAQVDPFSASIILFVGRAWDAITDPLVGFCISKSPWTRLGRLMPWITFSTPL
AIIAYFLIWFVPDFHQGQTLWYLLFYCLFETLVTCFHVPYSALTMFISTEQSERDSATAYRM
TVEVLGTVLGTAIQGQIVGQADSPCIPDANASTVNRTQSSTSIKETQNAYLLAAGVIASIYVI
CAVILILGVREQRESYETQQTKQMPFFRGLRLVMSHGPYIKLIAGFLFTSLAFMLVEGNFAL
FCTYTLGFRNEFQNLLLAIMFSATVTIPIWQWFLTRFGKKTAVYIGISSAVPFLILVALMESNL
IVTYVVAVAAGISVAAAFLLPWSMLPDVIDDFHLKQPHIHGTEPIFFSFYVFFTKFASGVSLG
ISTLSLDFTGYQTRGCSQPARVKFTLKMLVTMAPIVLILIGLLLFKLYPIDEEKRRQNKKALQ
ALREEASSSGCSDTDSTELASIL
SEQ ID NO: 46
mfsd2a_Armadillo
>MFSD2A_DASYPUS/1-528 PREDICTED: major facilitator superfamily domain-containing
protein 2A isoform 1 [Dasypus novemcinctus]
MAKGEGAESGSAAGLLPTGILQAGERPARVKKEPKKKQQLSICNKLCYAVGGAPYQVTG
CALGFFLQIYLLDVAQVNPFSASIILFVGRAWDAVTDPLVGFCISKTSWTRLGRLMPWIIFST
PLAVITYFLIWFVPDFPRHQALWYLLFYCLFETLVTCFHVPYSALTMFISTEQSERDSATAY
RMTVEVLGTVLGTAIQGQIVGQADTPCLQSPNDSTVTLEGANRTHSAISLKKTQNAYLLAA
GVIASIYVICAVILTLGVREQREPYETQKEPMSFFRGLRLVMSHGPYVKLIAGFLFTSLAFML
VEGNFALFCTYTLGFRNEFQNLLLVIMLSATFTIPFWQWFLTRFGKKTAVYIGISSAVPFLM
LVALMKSNLIVTYVVAVAAGISVAAAFLLPWSMLPDVVDDFHLKQPHSHGTEPIFFSFYVFF
TKFASGVSLGISTLSLDFAGYQTRGCSQPKLVRLTLKMLVTMAPIVLILLGLLLFKLYPIDEE
KRRQNRKALQALREEASSSGCSDTDSTELASIL
SEQ ID NO: 47
MFSD2B_Human
>MFSD2B_HUMAN/1-503 PREDICTED: major facilitator superfamily domain-containing
protein 2B isoform X1 [Homo sapiens]
MAAPPAPAAKGSQPEPHAPEPGPGSAKRGREDSRAGRLSFCTKVCYGIGGVPNQIASSA
TAFYLQLFLLDIAQIPAAQVSLVLFGGKVSGAAADPVAGFFINRSQRTGSGRLMPWVLGCT
PFIALAYFFLWFLPPFTSLRGLWYTTFYCLFQALATFFQVPYTALTMLLTPCPRERDSATAY
RMTVEMAGTLMGATVHGLIVSGAHRPHRCEATATPGPVTVSPNAAHLYCIAAAVVVVTYP
VCISLLCLGVKERPDPSAPASGPGLSFLAGLSLTTRHPPYLKLVISFLFISAAVQVEQSYLVL
FCTHASQLHDHVQGLVLTVLVSAVLSTPLWEWVLQRFGKKTSAFGIFAMVPFAILLAAVPT
APVAYVVAFVSGVSIAVSLLLPWSMLPDVVDDFQLQHRHGPGLETIFYSSYVFFTKLSGAC
ALGISTLSLEFSGYKAGVCKQAEEVVVTLKVLIGAVPTCMILAGLCILMVGSTPKTPSRDAS
SRLSLRRRTSYSLA
SEQ ID NO: 48
mfsd2b_mouse
>MFSD2B_MUS/1-494 major facilitator superfamily domain-containing protein 2B
[Mus musculus]
MSVPHGPTPAPVAEPHTQEPGSDKRDGRLSVCTKVCYGIGGVPNQVASSASAFYLQLFLL
DVAQIPAAQVSLALFGGKVSGAVADPVAGFFINKSRRTGSGRLMPWALGCMPLIALAYFFL
WFLPPFTSLRGLWYTSFYCLFQALATFFQVPYTALTMILTPSPRERDSATAYRMTMEMAG
TLMGATVHGLIVSSAHGSQRCEDTVHPRSPAVSPDVARLYCIAAAVVALTYPVCGSLLCLG
VKEQPDTSAPASGQGLNFFTGLAITSQHPPYLSLVVSFLFISAAVQVEQSYLVLFCTHASKL
QDHVQNLVLIILVSAVLSTPLWEWVLQRFGKKTSAFGICVMVPFSILLAAVPSAPVAYVVAF
VSGVSIAVSLLLPWSMLPDVVDDFQLQHRCGPGVETIFYSSYVFFTKLSGAGALGISTLSLE
FAGYEAGACQQAEEVVVTLKVLIGAVPTCMILIGLCILLVGPTPKMPRQDTSSQLSLRRRTS
YSLA
SEQ ID NO: 49
mfsd2b_Elefant
>MFSD2B_LOXODONTA/1-490 PREDICTED: major facilitator superfamily domain-
containing protein 2B [Loxodonta africana]
MPNIVLATGDAMLQCLPQDSRAGRLSFCTKLCYGIGGIPNQVASSATAFYLQLFLLDVAQIP
AAQVSLVLFGGKVSGAAADPVAGFFINRSRRTGSGRLMPWVLGCTPFIALAYFFLWFLPP
FTNLRGVWYMTFYCLFQALATFFQVPYTALTMLLTPNPRERDSATAYRITMEMAGTLMGA
TVHGLIVSRAHGPHRCQEAEIPVPDGVSPDAMRLYSIAAAVVAVTYPVCSGLLRLGVKERP
DPSTPASGQGLSFLAGLGLTVRHPPYLKLVVSFLFISAAVQVEQNYLVLFCTHASRLHDHV
QRLVLTILISAVLSTPLWEWVLHRFGKKTPAFGICVMVPFAILLAAVPVAPVAYVVAFVSGV
SIAVSLLLPWSMLPDVVDDFQLQHQHGPGLETIFYSSYVFFTKLSGASALGISTLSLEFVGY
KAGACEQTEEVVVTLKVLIGAVPTCMILTGLCILLVSRTPKVPSQDSSRQLSLRRRTSYSLA
SEQ ID NO: 50
mfsd2b_Dog
>MFSD2B_CANIS/1-453 PREDICTED: major facilitator superfamily domain-containing
protein 2B [Canis lupus familiaris]
MPNQVASSATAFYLQLFLLDVAQIPAAQVSLVLFGGKVSGAAADPVAGFFINRSSRTGSG
RLMPWVLGCTPFIAVAYFLLWFLPPFSSLRGLWYTAFYCVFQALATSLQVPYAALTMRLTP
CPRERDSATAYRMTMEMAGTLLGAAVHGLLVSGAHRPHPCEDAAPPGPQTDSADATRLY
SIAAAVVAMMYPVCSGLLCLGVKERPDSSAPASGQGLSFLAGLGLTVRHPPYLKLVISFLFI
SAAIQVEQSYLVLFCTHASRLHDHVQGVVLTILVSAVLSTPLWEWVLQRFGKKTSAFGICV
MVPFAILLAAVPTAPVAYVVAFVSGLSIAVSLLLPWSMLPDVVDDFQLQHQHSPGLETIFYS
SYVFFTKLSGAGALGISTLSLEFAGYEAGACKQAEQVVVTLKVLIGAVPTCMILTGLCILMA
GPAPKVPSQDPSRRLSLQRRTSYSLA
SEQ ID NO: 51
mfsd2b_Cow
>MFSD2B_BOS/1-480 PREDICTED: major facilitator superfamily domain-containing
protein 2B [Bos taurus] >gi|741943912|ref|XP_010808593.1|PREDICTED: major facilitator
superfamily domain-containing protein 2B [Bos taurus]
MGAELLCLPQDSGAGRLSFYRKLCYGIGGVPNQVASSAIAFYLQLFLLDVAQIPAAQVSLV
LFGGKVSGAAADPLAGFLINRSRRTGSGRLMPWVLGCMPFIALAYFFLWFLPPFTTLRGL
WYTTLYCLFQALATFFQVPYTALTMLLTPNPKERDSATAYRMTLEMAGTLMGATVHGLIVS
GAHGSHRCKEDTLPGEGAVSPNATRLYFIAATVVALTYPVCSTLLYLGVKERSDPSTPASG
QGLGFLAGLGLTVRHRPYLNLVISFLFISAAVQVEQSYLVLFCTHASQLQDHVQGMVLTILV
SAVLSTPMWEWVLQRFGKRMSALGICAMVPFAILLAAVPMVPVAYVVAFVSGLSIAVSLLL
PWSMLPDVVDDFQLQHQHGPGLETIFYSSYVFFTKLSGAGALGISTLSLDFAGYESGACR
QSEQVVVTLKVLIGAVPTSMILIGLCILMVGPTPKVPSRANSRSLGRRTSYTLA
SEQ ID NO: 52
mfsd2b_Armadillo
>MFSD2B_DASYPUS/1-505 PREDICTED: LOW QUALITY PROTEIN: major facilitator
superfamily domain-containing protein 2B [Dasypus novemcinctus]
MRPGQDAGRGGTVRLLRSQRLHPRERPGRPRGQDSGAGRLSFCTKLCYGIGGVPNQMA
SSATAFYLQLFLLDVAQIPAAQVSLVLCGGKVSGAAADPVAGFLINRSRRTGSGHLMPWV
LGCTPLAALAYFLLWLLPPAASLRGLWYTAFYCLFQALSTALHVPYAALTMLLTECPMERD
SATAYRMTMEMAGTLAGAAAHGLLVAGAHQPRGCHEAALPGPGGVSPHAARLYSIAAAV
VAAAYPVCSCLLCLGVKERPEPPGPDSGPGLGFLAGLGLTARHPPYLRLVVSFLLISAAVQ
VEQSYLVLFCTHAAQLHGHVQGLVLTVLVSAVLSTPLWEWVLQRFGKMTSXXGLCTGAP
GVRLTWSLPRLLTGWWPSRGNPGXLCPAPPPPRSMLPDVVDGFQRQHPHGPGLETIFY
SSYVFFTKLSGAGALGISTLSLEFAGYKAGACEQVEEVAVTLKVLISAVPTCMILAGLCVLR
AGPRPEVPGPASARQLSLRRRTSYSLA
Sequences in FIG. 12:
SEQ ID NO: 53
Wild type human SYNCYTIN 2. Referred to in the Examples as SYNC2_{WT}
NCBI Reference Sequence: NP_997465.1
https://www.ncbi.nlm.nih.gov/protein/NP_997465.1 ?report=genbank&log$=prottop&blast_
rank=1&RID=VRAT57J0013
Uniprot ID: P60508
>SYNC2_{WT}_Human
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVMAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQSI
HQCLPSNWTGTCTIGYVTPDIFIAPGNLSLPIPIYGNSPLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPLTGP
LVSLLLLLLFGPCLLNLITQFVSSRLQAIKLQTNLSAGRHPRNIQESPF
SEQ ID NO: 54
Sync2_Chimpazee
>gi|124244042|ref|NP_001074253.1|/1-538 syncytin-2 precursor [Pan troglodytes]\
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSMVKQDFPDIRQKPPIFGPIFTNINLMGIA
PICVMAKRKNGTNVGTLPSTVCNVTFTVDSNQQTYQTYTHNQFRHQPRFPKPPNITFPQG
TLLDKSSRFCQGRPSSCSTRNFWFRPADYNQCLQISNLSSTAEWVLLDQTRNSLFWENK
TKGANQSQTPCVQVLAGMTIATSYLGISAVSEFFGTSLTPLFHFHISTCLKTQGAFYICGQS
IHQCLPSNWTGTCTIGYVTPDIFIAPGNLSLPIPIYGNSQLPRVRRAIHFIPLLAGLGILAGTG
TGIAGITKASLTYSQLSKEIANNIDTMAKALTTMQEQIDSLAAVVLQNRRGLDMLTAAQGGI
CLALDEKCCFWVNQSGKVQDNIRQLLNQASSLRERATQGWLNWEGTWKWFSWVLPLTG
PLVSLLLLLLFGPCLLNLITQFVSSRLQAIKLQTNLSAGRHPRNIQESPF
SEQ ID NO: 55
Sync2_Gorilla
>gi|47605605|sp|P61554.1|SYCY2_GORGO/1-538 [Gorilla gorilla]\
MGLLLLVLILTPSLAAYRHPDFPLLEKAQQLLQSTGSPYSTNCWLCTSSSTETPGTAYPAS
PREWTSIEAELHISYRWDPNLKGLMRPANSLLSTVKQDFPDIRQKPPIFGPIFTNINLMGIAP
ICVTAKRKNGTNVGTLPSTVCNVTFTVDSNQQTYQTYTHNQFRHQPRFPKPPNITFPQGT
LLDKSSRFCQGRPSSCSTRNFWFRPADYNQCLQISNLSSTAEWVLLDQTRNSLFWENKT
KGANQSQTPCVQVLAGMTIATSYLGISAVSEFFGTSLTPLFHFHISTCLKTQGAFYICGQLI
HQCLPSNWTGTCTIGYVTPDIFIAPGNLSLPIPIYGNSQLPRVRRAIHFIPLLAGLGILAGTGT
GIAGITKASLTYSQLSKEIANNIDTMAKALTTMQEQIDSLAAVVLQNRRGLDMLTAAQGGIC
LALDEKCCFWVNQSGKVQDNIRQLLNQASSLRERATQGWLNWEGTWKWFSWVLPLTGP
LVSLLLLLLFGPCLLNLITQFVSSRLQAIKLQTNLSAGRRPRNIQESPF
SEQ ID NO: 56
Sync2_Orangutan
>gi|758371911|SYCY2_PONPY/1-538 [Pongo pygmaeus]\
MGLLLLVLILTPLLAAHRHPDFPLLEKAQQLLQSTGSPYSTNCWLCTSSSTETPGTAYPAS
PREWTSIEAELHISYHWDPNLKGLMRPANSLLSTVKQDFPDIRQKPPIFGPIFTNINLMGIAP
ICVTAKRKNGTNVGTLPSTVCNVTFTVDPNQQTYQTYTHKQFLHQPRFPKPPNITFPQGTL
LDKSTRFCQGRPSSCSTRNFWFRPADYNQCLQISNLSSTAEWVLLDQTRNSLFWENKTK
GANQSQTPCVQVLAGMTIATSYLGISAVSEFFGTSLTPLFHFHISTCLKTQGAFYICGQSIH
QCLPSNWTGTCTIGYVTPDIFIAPGNISLPIPIYGNSQLPRVRRAIHFIPLLAGLGIIAGTGTGI
AGITKASLTYSQLSKEIAKNIDTMAKALTTVQEQIDSLAAVVLQNRRGLDMLTAAQGGICLA
LDEKCCFWVNQSGKVQDNIRQLLNQASSLRERATQGWLNWEGTWKWFSWVLPFTGPLV
SLLLLLLFGPCLLNLITQFVLSRLQAIKLQTNLSAGCRPHNIQESPF
SEQ ID NO: 57
Sync2_Gibbon
>gi|758371894|ref|NP_001292001.1|/1-538 syncytin-2 precursor [Nomascus leucogenys]\
MGLLLLVLILTPLLAAYRHPDFPLLEKAQQLLQSTGSPYSTNCWLCTSSSTETPGTAYPAS
PREWTSIEAELHISYQWDPNLKGLMRPANSLLSTVKQDFPDIRQKPPIFGPIFTNINLMGIAP
ICVTAKRKNGTNVGTLPSTVCNVTFTVDPNQQTYQTYTHNQFRHQPRFPKPPNITFPQGT
LLDKSTRFCQGRPSSCSTRNFWFRPADYNQCLQISNLSSTAEWVLLDQTRNSLFWENKT
KGANQSQTPCVQVLAGMTIATSYLGISAVSEFFGNSLTPLFHFHISTCLKTQGAFYICGQSI
HQCLPSNWTGTCTIGYVTPDIFIAPGNLSLPIPIYGKSQLPRVRRAIHFIPLLAGLGILAGTGT
GIAGITKASLTYSQLSKEIANNIDTMAKTLTTVQEQIDSLAAVVLQNRRGLDMLTAAQGGICL
ALDEKCCFWVNQSGKVQDNIRQLLNQASSLRERATQGWLNWEGTWKWFSWVLPFIGPL
VSLLLLLLFGPCLLNLITQFVSSRLQAIKLQTNLSAGRRPRTIQESPF
SEQ ID NO: 58
Sync2_Macaques
>gi|806776650|ref|NP_001292976.1|/1-537 syncytin-2 precursor [Macaca nemestrina]\
MGLLLLVLILTPLLAAYRHPDFPLLEKAQQLLQSTGSPYSTNCWLCTSSSTETPGTAYPAS
PREWTSIEAELHISYQWDPNLKGLMTPANSLLSTVKQDFPDIRQKPPIFGPIFTNINLMGKA
PICVTAKRKNGTNVGTLPSTVCNVTFTVDPNQQTYQTYTHNQFRHQPRFPKPPNITFPQG
TLLDKSTQFCQGRPSSCRTRNFWFRPADYNQCLQIPKLSSPAEWVLLDQTRNSLFWENK
TKGANQSQTPCIQVLAGMTIATSYLGISAVSEFFGTSLTPLFHFHISTCLKTQGAFYICGQSI
HQCLPTNWTGTCTIGYVTPDIFIAPGNLSLPIPIYGNSQLPRVRRAIHFIPLLAGLGILAGTGT
GIAGITKASFTYSQLSKEIAKNIDTMAKTLTTVQEQIDSLAAVVLQNRRGLDMLTAAQGGICL
ALDEKCCFWVNQSGKVQDNIRQLLNQASSLRERATQGWLNWEGTWKWFSWVLPFIGPL
VSLLLLLLFGPCLLNLITQFVSSRLQAIKLQTNGAGCRPRNIQESPF
SEQ ID NO: 59
Sync2_Baboon
>gi|758371886|ref|NP_001291999.1|/1-537 syncytin-2 precursor [Papio anubis]\
MGLLLLVLILTPLLAAYRHPDFPLLEKAQQLLQSTGSPYSTNCWLCTSSSTEMPGTAYPAS
PREWTSIEAELHISYQWDPNLKGLMTPANSLLSTVKQDFPDIRQKPPIFGPIFTNINLMGKA
PICVTAKRKNGTNVGTLPSTVCNVTFTVDPNQQTYQTYTHNQFRHQPRFPKPPNITFPQG
TLLDKSTQFCQGRPSSCRTRNFWFRPADYNQCLQIPNLSSPAEWVLLDQTRNSLFWENK
TKGANQSQTPCIQVLAGMTIATSYLGISAVSEFFGTSLTPLFHFHISTCLKTQGAFYICGQSI
HQCLPTNWTGTCTIGYVTPDIFIAPGNLSFPIPIYGNSQLPRVRRAIHFIPLLAGLGILAGTGT
GIAGITKASFTYSQLSKEIAKNIDTMAKTLTTVQEQIDSLAAVVLQNRRGLDMLTAAQGGICL
ALDEKCCFWVNQSGKVQDNIRQLLNQASSLRERATQGWLNWEGTWKWFSWVLPFIGPL
VSLLLLLLFGPCLLNLITQFVSSRLQAIKLQTNGAGCRPRNIQESTF
SEQ ID NO: 60
Sync2_green_monkey
>gi|758818516|ref|NP_001292027.1|/1-537 syncytin-2 precursor [Chlorocebus sabaeus]\
MGLLLLVLILTPLLAAYRHPDFSLLEKAQQLLQSTGSPYSTNCWLCTRSSTETPGTAYPAS
PREWTSIEAELHISYQWDPNLKGLMTPANSLLSTVKQDFPDIRQKPPIFGPIFTNINLMGKA
PICVTAKRKNGTNVGTLPSTVCNVTFTVDPHQQTYQTYTHNQFRHQPRFPKPPNITFPQG
TLLDKSTQFCQGRPSSCRTRNFWFRPADYNQCLQIPNLSSPAEWVLLDQTRNSLFWENK
TKGANQSQTPCIQVLAGMTIATSYLGISAVSEFFGTSLTPLFHFHISTCLKTQGAFYICGQSI
HQCLPTNWTGTCTIGYVTPDIFIAPGNLSLPIPIYGNSQLPRVRRAIHFIPLLAGLGILAGTGT
GIAGITKASFTYSQLSKEIAKNIDTLAKTLTTVQEQIDSLAAVVLQNRRGLDMLTAAQGGICL
ALDEKCCFWVNQSGKVQDNIRQLLNQASSLRERATQGWLNWEGTWKWFSWVLPFIGPL
VSLLLLLLFGPCLLNLITQFVSSRLQAIKLQTNSAGCRPRNIQESPF
SEQ ID NO: 61
Sync2_Golden_s.nosed
>gi|758818504|ref|NP_001292026.1|/1-537 syncytin-2 precursor [Rhinopithecus roxellana]\
MGLLLLVLILTPLLAAYRHPDFPLLEKAQQLLQSTGSPYSTSCWLCTSSSTETPGTAYPASP
REWTSIEAELHISYQWDPNLKGLMTPANSLLSTVQQDFPDIRQKPPIFGPIFTNINLMGKAPI
CVTAKRKNGTNVGTLPSTVCNVTFTVDPNQQTYQTYTHNQFRHQPRFPKPPNITFPQGTL
LDKSTQFCQGRPSSCRTRNFWFRPADYNQCLQIPNLSSPAEWVLLDQTRNSLFWENKTK
GANQSQTPCVQVLAGMTIATSYLGISAVSEFFGTSLTPLFHFHISTCLKTQGAFYICGQSIH
QCLPTNWTGTCTIGYVTPDIFIAPGNLSLPIPIYGNSQLPRVRRAIHFIPLLAGLGILAGTGTG
IAGITKASFTYSQLSKEIAKNIDTMAKTLTTVQEQIDSLAAVVLQNRRGLDMLTAAQGGICLA
LDEKCCFWVNQSGKVQDNIRQLLNQASSLRERATQGWLNWEGTWKWFSWVLPFIGPLV
SLLLLLLFGPCLLNLITQFVSSRLQAIKLQTNSARCRPRNIQESPF
SEQ ID NO: 62
Sync2_Angolan_colubus
>gi|806776487|ref|NP_001292974.1|/1-544 syncytin-2 precursor [Colobus angolensis
palliatus]\
MGLLLLVLILTPLLAAYRHPDFPLLEKAQQLLQSTRSPYSTNCWLCTSSSTKTPGTAYPASP
REWTSIEAELHISYQWDPNLKGLMTPANSLLSTVKQDFPDIRQKPPIFGPIFTNINLMGKAPI
CVTAKRKNGTNVGTLPSTVCNVTFTVDPNQQTYQTYTHNQFRHQPRFPKPPNITFPQGTL
LDKSTQFCQGRPSSCRTRNFWFRPADYNQCLQIPNLSSPAEWVLLDQTRNSLFWENKTN
GANQSQTPCVQVLAGMTIATSYLGISAVSEFFGTSLTPLFHFHISTCLKTQGAFYICGQSIH
QCLPTNWTGTCTIGYVTPDIFIAPGNLSLPIPIYGNSQLPRVRRAIHFIPLLAGLGILAGTGTG
IAGITKASFTYSQLSKEIAKNIDTMAQTLTTVQEQIDSLAAVVLQNRRGLDMLTAAQGGICLA
LDEKCCFWVNQSGKVQDNIRQLLNQASSLRERATQGWLNWEGTWKWFSWVLPFIGPLV
SLLLLLLFGPCLLNLITQFVSSRLQAIKLQTNSARCRPRNIQESPF
SEQ ID NO: 63
Sync2_marmoset
>gi|758818520| ref|NP_001292025.1//1-545 syncytin-2 precursor [Callithrix jacchus]\
MGLLLLLLILTPLLAAYRHPDFRLLEKAQQLLQSTGSPYSTNCWLCTSSSTKTPGRAYPAS
SREWTTIEAELHISYQWDPNLKGLIKPANSLLSKVKQDFPDIRKEPPIFGPIFTNVNLIGIAPI
CVTAKRKDGTNVGTLPSTVCNVTLTVDPNQQTYQKYAHNQFHHQPRFPKPPNITFPQGTL
LDKSTRFCQGRPSSCSTRNFWFQPADYNQCLQIPNLSSTAEWVLLDQTRNSLFWENKTK
GANQSQTPCVQVLAGMTIATSYLSISAVSEFSGTSVTSLFSFHISTCLKTQGAFYICGQSIH
QCLPTNWTGTCTIGYVSPDIFIAPGNLSLPIPIYGNSHFPRVRRAIHLIPLLVGLGIVGSAGTG
IAGIAKASFTYSQLSKEIANNIEAMAKTLTTVQEQIDSLAAVVLQNRRGLDMLTAAQGGICLA
LEEKCCFWVNQSGKVQDNIRQLLNRASTLQEQATQGWLSWEGTWKWFSWVLPFTGPLV
SLLLLLLFGPCLLNLITQFVSSRLQATKLQMKLNKRVRPRNSQESPF

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallogr D Biol Crystallogr 66, 213-221.
Alakbarzade, V., Hameed, A., Quek, D. Q., Chioza, B. A., Baple, E. L., Cazenave-Gassiot, A., Nguyen, L. N., Wenk, M. R., Ahmad, A. Q., Sreekantan-Nair, A., et al. (2015). A partially inactivating mutation in the sodium-dependent lysophosphatidylcholine transporter MFSD2 Å causes a non-lethal microcephaly syndrome. Nat Genet 47, 814-817.
Andreone, B. J., Chow, B. W., Tata, A., Lacoste, B., Ben-Zvi, A., Bullock, K., Deik, A. A., Ginty, D. D., Clish, C. B., and Gu, C. (2017). Blood-Brain Barrier Permeability Is Regulated by Lipid Transport-Dependent Suppression of Caveolae-Mediated Transcytosis. Neuron 94, 581-594 e585.
Ben-Zvi, A., Lacoste, B., Kur, E., Andreone, B. J., Mayshar, Y., Yan, H., and Gu, C. (2014). Mfsd2a is critical for the formation and function of the blood-brain barrier. Nature 509, 507-511.
Blaise, S., de Parseval, N., Benit, L., and Heidmann, T. (2003). Genomewide screening for fusogenic human endogenous retrovirus envelopes identifies syncytin 2, a gene conserved on primate evolution. Proc Natl Acad Sci USA 100, 13013-13018.
Blond, J. L., Lavillette, D., Cheynet, V., Bouton, O., Oriol, G., Chapel-Fernandes, S., Mandrand, B., Mallet, F., and Cosset, F. L. (2000). An envelope glycoprotein of the human endogenous retrovirus HERV-W is expressed in the human placenta and fuses cells expressing the type D mammalian retrovirus receptor. J Virol 74, 3321-3329.
Cabantous, S., Terwilliger, T. C., and Waldo, G. S. (2005). Protein tagging and detection with engineered self-assembling fragments of green fluorescent protein. Nat Biotechnol 23, 102-107.
Canul-Tec, J. C., Assal, R., Cirri, E., Legrand, P., Brier, S., Chamot-Rooke, J., and Reyes, N. (2017). Structure and allosteric inhibition of excitatory amino acid transporter 1. Nature 544, 446-451.
Cater, R. J., Chua, G. L., Erramilli, S. K., Keener, J. E., Choy, B. C., Tokarz, P., Chin, C. F., Quek, D. Q. Y., Kloss, B., Pepe, J. G., et al. (2021). Structural basis of omega-3 fatty acid transport across the blood-brain barrier. Nature.
Chen, C. P., Chen, L. F., Yang, S. R., Chen, C. Y., Ko, C. C., Chang, G. D., and Chen, H. (2008). Functional characterization of the human placental fusogenic membrane protein syncytin 2. Biol Reprod 79, 815-823.
Chow, B. W., and Gu, C. (2015). The molecular constituents of the blood-brain barrier. Trends Neurosci 38, 598-608.
Chow, B. W., and Gu, C. (2017). Gradual Suppression of Transcytosis Governs Functional Blood-Retinal Barrier Formation. Neuron 93, 1325-1333 e1323.
Cirri, E., Brier, S., Assal, R., Canul-Tec, J. C., Chamot-Rooke, J., and Reyes, N. (2018). Consensus designs and thermal stability determinants of a human glutamate transporter. Elife 7.
Collaborators, G. B. D. N. (2019). Global, regional, and national burden of neurological disorders, 1990-2016: a systematic analysis for the Global Burden of Disease Study 2016. Lancet Neurol 18, 459-480.
Cui, L., Wang, H., Lu, X., Wang, R., Zheng, R., Li, Y., Yang, X., Jia, W. T., Zhao, Y., Wang, Y., et al. (2016). Effects of individually silenced N-glycosylation sites and non-synonymous single-nucleotide polymorphisms on the fusogenic function of human syncytin-2. Cell Adh Migr 10, 39-55.
Drozdetskiy, A., Cole, C., Procter, J., and Barton, G. J. (2015). JPred4: a protein secondary structure prediction server. Nucleic Acids Res 43, W389-394.
Edgar, R. C. (2004). MUSCLE: multiple sequence alignment with high accuracy and high throughput. Nucleic Acids Res 32, 1792-1797.
Emsley, P., Lohkamp, B., Scott, W. G., and Cowtan, K. (2010). Features and development of Coot. Acta Crystallogr D Biol Crystallogr 66, 486-501.
Esnault, C., Priet, S., Ribet, D., Vernochet, C., Bruls, T., Lavialle, C., Weissenbach, J., and Heidmann, T. (2008). A placenta-specific receptor for the fusogenic, endogenous retrovirus-derived, human syncytin-2. Proc Natl Acad Sci USA 105, 17532-17537.
Ethayathulla, A. S., Yousef, M. S., Amin, A., Leblanc, G., Kaback, H. R., and Guan, L. (2014). Structure-based mechanism for Na(+)/melibiose symport by MeIB. Nat Commun 5, 3009.
Glaser, F., Pupko, T., Paz, I., Bell, R. E., Bechor-Shental, D., Martz, E., and Ben-Tal, N. (2003). ConSurf: identification of functional regions in proteins by surface-mapping of phylogenetic information. Bioinformatics 19, 163-164.
Gong, X., Qian, H., Zhou, X., Wu, J., Wan, T., Cao, P., Huang, W., Zhao, X., Wang, X., Wang, P., et al. (2016). Structural Insights into the Niemann-Pick C1 (NPC1)-Mediated Cholesterol Transfer and Ebola Infection. Cell 165, 1467-1478.
Guemez-Gamboa, A., Nguyen, L. N., Yang, H., Zaki, M. S., Kara, M., Ben-Omran, T., Akizu, N., Rosti, R. O., Rosti, B., Scott, E., et al. (2015). Inactivating mutations in MFSD2A, required for omega-3 fatty acid transport in brain, cause a lethal microcephaly syndrome. Nat Genet 47, 809-813.
Harel, T., Quek, D. Q. Y., Wong, B. H., Cazenave-Gassiot, A., Wenk, M. R., Fan, H., Berger, I., Shmueli, D., Shaag, A., Silver, D. L., et al. (2018). Homozygous mutation in MFSD2A, encoding a lysolipid transporter for docosahexanoic acid, is associated with microcephaly and hypomyelination. Neurogenetics 19, 227-235.
Harrison, S. C. (2005). Mechanism of membrane fusion by viral envelope proteins. Adv Virus Res 64, 231-261.
Holm, L. (2020). DALI and the persistence of protein shape. Protein Sci 29, 128-140.
Horrocks, L. A., and Yeo, Y. K. (1999). Health benefits of docosahexaenoic acid (DHA). Pharmacol Res 40, 211-225.
Kaddoum, L., Magdeleine, E., Waldo, G. S., Joly, E., and Cabantous, S. (2010). One-step split GFP staining for sensitive protein detection and localization in mammalian cells. Biotechniques 49, 727-728, 730, 732 passim.
Kidd, P. M. (2007). Omega-3 DHA and EPA for cognition, behavior, and mood: clinical findings and structural-functional synergies with cell membrane phospholipids. Altern Med Rev 12, 207-227.
Kobayashi, N., Kawasaki-Nishi, S., Otsuka, M., Hisano, Y., Yamaguchi, A., and Nishi, T. (2018). MFSD2B is a sphingosine 1-phosphate transporter in erythroid cells. Sci Rep 8, 4969.
Krissinel, E., and Henrick, K. (2007). Inference of macromolecular assemblies from crystalline state. J Mol Biol 372, 774-797.
Kumar, A., Planchais, C., Fronzes, R., Mouquet, H., and Reyes, N. (2020). Binding mechanisms of therapeutic antibodies to human CD20. Science 369, 793-799.
Lavialle, C., Cornelis, G., Dupressoir, A., Esnault, C., Heidmann, O., Vernochet, C., and Heidmann, T. (2013). Paleovirology of ‘syncytins’, retroviral env genes exapted for a role in placentation. Philos Trans R Soc Lond B Biol Sci 368, 20120507.
Mallet, F., Bouton, O., Prudhomme, S., Cheynet, V., Oriol, G., Bonnaud, B., Lucotte, G., Duret, L., and Mandrand, B. (2004). The endogenous retroviral locus ERVWE1 is a bona fide gene involved in hominoid placental physiology. Proc Natl Acad Sci USA 101, 1731-1736.
Mastronarde, D. N. (2005). Automated electron microscope tomography using robust prediction of specimen movements. J Struct Biol 152, 36-51.
Mi, S., Lee, X., Li, X., Veldman, G. M., Finnerty, H., Racie, L., LaVallie, E., Tang, X. Y., Edouard, P., Howes, S., et al. (2000). Syncytin is a captive retroviral envelope protein involved in human placental morphogenesis. Nature 403, 785-789.
Nguyen, L. N., Ma, D., Shui, G., Wong, P., Cazenave-Gassiot, A., Zhang, X., Wenk, M. R., Goh, E. L., and Silver, D. L. (2014). Mfsd2a is a transporter for the essential omega-3 fatty acid docosahexaenoic acid. Nature 509, 503-506.
O'Brown, N. M., Megason, S. G., and Gu, C. (2019). Suppression of transcytosis regulates zebrafish blood-brain barrier function. Elife 8.
Ozorowski, G., Pallesen, J., de Val, N., Lyumkis, D., Cottrell, C. A., Torres, J. L., Copps, J., Stanfield, R. L., Cupo, A., Pugach, P., et al. (2017). Open and closed structures reveal allostery and pliability in the HIV-1 envelope spike. Nature 547, 360-363.
Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J Comput Chem 25, 1605-1612.
Punjani, A., Rubinstein, J. L., Fleet, D. J., and Brubaker, M. A. (2017). cryoSPARC: algorithms for rapid unsupervised cryo-EM structure determination. Nat Methods 14, 290-296.
Punjani, A., Zhang, H., and Fleet, D. J. (2020). Non-uniform refinement: adaptive regularization improves single-particle cryo-EM reconstruction. Nat Methods 17, 1214-1221.
Quek, D. Q., Nguyen, L. N., Fan, H., and Silver, D. L. (2016). Structural Insights into the Transport Mechanism of the Human Sodium-dependent Lysophosphatidylcholine Transporter MFSD2A. J Biol Chem 291, 9383-9394.
Quistgaard, E. M., Low, C., Guettou, F., and Nordlund, P. (2016). Understanding transport by the major facilitator superfamily (MFS): structures pave the way. Nat Rev Mol Cell Biol 17, 123-132.
Renard, M., Varela, P. F., Letzelter, C., Duquerroy, S., Rey, F. A., and Heidmann, T. (2005). Crystal structure of a pivotal domain of human syncytin-2, a 40 million years old endogenous retrovirus fusogenic envelope gene captured by primates. J Mol Biol 352, 1029-1034.
Rey, F. A., and Lok, S. M. (2018). Common Features of Enveloped Viruses and Implications for Immunogen Design for Next-Generation Vaccines. Cell 172, 1319-1334.
Robbins, J. R., and Bakardjiev, A. I. (2012). Pathogens and the placental fortress. Curr Opin Microbiol 15, 36-43.
Roberts, R. M., Ezashi, T., Schulz, L. C., Sugimoto, J., Schust, D. J., Khan, T., and Zhou, J. (2021). Syncytins expressed in human placental trophoblast. Placenta.
Ruigrok, K., Vaney, M. C., Buchrieser, J., Baquero, E., Hellert, J., Baron, B., England, P., Schwartz, O., Rey, F. A., and Backovic, M. (2019). X-ray Structures of the Post-fusion 6-Helix Bundle of the Human Syncytins and their Functional Implications. J Mol Biol 431, 4922-4940.
Scala, M., Chua, G. L., Chin, C. F., Alsaif, H. S., Borovikov, A., Riazuddin, S., Riazuddin, S., Chiara Manzini, M., Severino, M., Kuk, A., et al. (2020). Biallelic MFSD2 Å variants associated with congenital microcephaly, developmental delay, and recognizable neuroimaging features. Eur J Hum Genet 28, 1509-1519.
Shaik, M. M., Peng, H., Lu, J., Rits-Volloch, S., Xu, C., Liao, M., and Chen, B. (2019). Structural basis of coreceptor recognition by HIV-1 envelope spike. Nature 565, 318-323.
Slabinski, L., Jaroszewski, L., Rychlewski, L., Wilson, I. A., Lesley, S. A., and Godzik, A. (2007). XtalPred: a web server for prediction of protein crystallizability. Bioinformatics 23, 3403-3405.
Terstappen, G. C., Meyer, A. H., Bell, R. D., and Zhang, W. (2021). Strategies for delivering therapeutics across the blood-brain barrier. Nat Rev Drug Discov 20, 362-383.
Terwilliger, T. C., Sobolev, O. V., Afonine, P. V., and Adams, P. D. (2018). Automated map sharpening by maximization of detail and connectivity. Acta Crystallogr D Struct Biol 74, 545-559.
Tian, W., Chen, C., Lei, X., Zhao, J., and Liang, J. (2018). CASTp 3.0: computed atlas of surface topography of proteins. Nucleic Acids Res 46, W363-W367.
Vu, T. M., Ishizu, A. N., Foo, J. C., Toh, X. R., Zhang, F., Whee, D. M., Torta, F., Cazenave-Gassiot, A., Matsumura, T., Kim, S., et al. (2017). Mfsd2b is essential for the sphingosine-1-phosphate export in erythrocytes and platelets. Nature 550, 524-528.
Wang, J. Z., Xiao, N., Zhang, Y. Z., Zhao, C. X., Guo, X. H., and Lu, L. M. (2016). Mfsd2a-based pharmacological strategies for drug delivery across the blood-brain barrier. Pharmacol Res 104, 124-131.
Watanabe, Y., Berndsen, Z. T., Raghwani, J., Seabright, G. E., Allen, J. D., Pybus, O. G., McLellan, J. S., Wilson, I. A., Bowden, T. A., Ward, A. B., et al. (2020). Vulnerabilities in coronavirus glycan shields despite extensive glycosylation. Nat Commun 11, 2688.
Waterhouse, A. M., Procter, J. B., Martin, D. M., Clamp, M., and Barton, G. J. (2009). Jalview Version 2—a multiple sequence alignment editor and analysis workbench. Bioinformatics 25, 1189-1191.
Wong, B. H., Chan, J. P., Cazenave-Gassiot, A., Poh, R. W., Foo, J. C., Galam, D. L., Ghosh, S., Nguyen, L. N., Barathi, V. A., Yeo, S. W., et al. (2016). Mfsd2a Is a Transporter for the Essential omega-3 Fatty Acid Docosahexaenoic Acid (DHA) in Eye and Is Important for Photoreceptor Cell Development. J Biol Chem 291, 10501-10514.
Wong, B. H., and Silver, D. L. (2020). Mfsd2a: A Physiologically Important Lysolipid Transporter in the Brain and Eye. Adv Exp Med Biol 1276, 223-234.
Wood, C. A. P., Zhang, J., Aydin, D., Xu, Y., Andreone, B. J., Langen, U. H., Dror, R. O., Gu, C., and Feng, L. (2021). Structure and mechanism of blood-brain-barrier lipid transporter MFSD2A. Nature.
Yan, N. (2015). Structural Biology of the Major Facilitator Superfamily Transporters. Annu Rev Biophys 44, 257-283.
Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
Yang, A. C., Stevens, M. Y., Chen, M. B., Lee, D. P., Stahli, D., Gate, D., Contrepois, K., Chen, W., Iram, T., Zhang, L., et al. (2020). Physiological blood-brain transport is impaired with age by a shift in transcytosis. Nature 583, 425-430.
Zhang, B., Liu, X., Lambert, E., Mas, G., Hiller, S., Veening, J. W., and Perez, C. (2020). Structure of a proton-dependent lipid transporter involved in lipoteichoic acids biosynthesis. Nat Struct Mol Biol 27, 561-569.
Zlokovic, B. V. (2008). The blood-brain barrier in health and chronic neurodegenerative disorders. Neuron 57, 178-201

1-23. (canceled)

24. A polypeptide-drug conjugate comprising a polypeptide covalently linked to a drug, directly or through a linker, wherein the drug is a small molecule, an antibody, a DNA, or a RNA, a liposome, or a nanoparticle, and wherein the polypeptide comprises an isolated syncytin 2 (SYNC2) polypeptide fragment.

25. The polypeptide-drug conjugate of claim 24, wherein the polypeptide comprises:
(i) a polypeptide of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(ii) a polypeptide whose sequence is at least 95% identical to SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(iii) a polypeptide having 1 or more amino acid substitutions, deletions, or additions relative to an amino acid sequence selected from SEQ ID NOs 3-24, but still having at least 85% identity to SYNC2 (SEQ ID NO:3); or
(iv) a soluble fragment of any one of (i)-(iii), wherein the fragment is at least 10 amino acid residues long, but shorter than 350.

26. The polypeptide-drug conjugate of claim 24, wherein the polypeptide comprises SYNC2_{SU-CO} (SEQ ID NO. 7), SYNC2_{SU-CO-2} (SEQ ID NO. 8), SYNC2_{SU-CO-3} (SEQ ID NO: 9), SYNC2_{SU-CO} (c43) (SEQ ID NO: 10), SYNC2_{SU-CO-2} (c43) (SEQ ID NO: 11), SYNC2_{SU-CO-3} (c43) (SEQ ID NO: 12), SYNC2_{SU-WT} (SEQ ID NO: 13), SYNC2_{SU-WT-2} (SEQ ID NO. 14), SYNC2_{SU-WT-3} (SEQ ID NO: 15), SYNC2_{SU-C43S} (SEQ ID NO: 16), SYNC2_{SU-C43S-2} (SEQ ID NO: 17), SYNC2_{SU-C43S-3} (SEQ ID NO: 18), RBL1 (SEQ ID NO: 19), RBL2 (SEQ ID NO: 20), RBL3 (SEQ ID NO: 21), RBLs4 (SEQ ID NO: 22), RBLs5 (SEQ ID NO: 23), or RBLs6 (SEQ ID NO: 24).

27. The polypeptide-drug conjugate of claim 24, wherein the polypeptide comprises SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24.

28. The polypeptide-drug conjugate of claim 24, wherein the polypeptide binds MFSD2 Å with a Ko of at least about 1×10⁻⁷ M.

29. A nucleic acid encoding the polypeptide-drug conjugate of claim 24, wherein the nucleic acid comprises any one of SEQ ID NOs: 25 through 40 and codon-optimized versions of the same.

30. A vector comprising the nucleic acid of claim 29.

31. An isolated cell comprising the polypeptide-drug conjugate of claim 24.

32. A pharmaceutical composition comprising the polypeptide-drug conjugate of claim 24 and a pharmaceutically acceptable carrier, diluent, or excipient.

33. A method of transiently inhibiting MFSD2 Å transport function in the blood brain barrier (BBB) or transiently increasing vesicular transcytosis across BBB endothelium in a subject in need thereof, comprising administering to the subject an effective dose of an isolated syncytin 2 (SYNC2) polypeptide fragment.

34. A method of transiently increasing BBB permeability to deliver therapeutic small-compounds, macromolecules in particular antibodies, nanomaterials in particular liposomes or gene therapy, into the brain of a subject in need thereof, comprising administering to the subject an effective dose of an isolated syncytin 2 (SYNC2) polypeptide fragment.

35. The method of claim 33, wherein the isolated syncytin 2 (SYNC2) polypeptide fragment comprises:
(i) a polypeptide of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(ii) a polypeptide whose sequence is at least 95% identical to SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(iii) a polypeptide having 1 or more amino acid substitutions, deletions, or additions relative to an amino acid sequence selected from SEQ ID NOs 3-24, but still having at least 85% identity to SYNC2 (SEQ ID NO:3); or
(iv) a soluble fragment of any one of (i)-(iii), wherein the fragment is at least 10 amino acid residues long, but shorter than 350.

36. The method of claim 33, wherein the isolated syncytin 2 (SYNC2) polypeptide fragment comprises SYNC2_{SU-CO} (SEQ ID NO. 7), SYNC2_{SU-CO-2} (SEQ ID NO. 8), SYNC2_{SU-CO-3} (SEQ ID NO: 9), SYNC2_{SU-CO} (c43) (SEQ ID NO: 10), SYNC2_{SU-CO-2} (c43) (SEQ ID NO: 11), SYNC2_{SU-CO-3} (c43) (SEQ ID NO: 12), SYNC2_{SU-WT} (SEQ ID NO: 13), SYNC2_{SU-WT-2} (SEQ ID NO. 14), SYNC2_{SU-WT-3} (SEQ ID NO: 15), SYNC2_{SU-C43S} (SEQ ID NO: 16), SYNC2_{SU-C43S-2} (SEQ ID NO: 17), SYNC2_{SU-C43S-3} (SEQ ID NO: 18), RBL1 (SEQ ID NO: 19), RBL2 (SEQ ID NO: 20), RBL3 (SEQ ID NO: 21), RBLs4 (SEQ ID NO: 22), RBLs5 (SEQ ID NO: 23), or RBLs6 (SEQ ID NO: 24).

37. The method of claim 33, wherein the isolated syncytin 2 (SYNC2) polypeptide fragment comprises SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24.

38. The method of claim 34, wherein the isolated syncytin 2 (SYNC2) polypeptide fragment comprises:
(i) a polypeptide of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(ii) a polypeptide whose sequence is at least 95% identical to SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24;
(iii) a polypeptide having 1 or more amino acid substitutions, deletions, or additions relative to an amino acid sequence selected from SEQ ID NOs 3-24, but still having at least 85% identity to SYNC2 (SEQ ID NO:3); or
(iv) a soluble fragment of any one of (i)-(iii), wherein the fragment is at least 10 amino acid residues long, but shorter than 350.

39. The method of claim 34, wherein the isolated syncytin 2 (SYNC2) polypeptide fragment comprises SYNC2_{SU-CO} (SEQ ID NO. 7), SYNC2_{SU-CO-2} (SEQ ID NO. 8), SYNC2_{SU-CO-3} (SEQ ID NO: 9), SYNC2_{SU-CO} (c43) (SEQ ID NO: 10), SYNC2_{SU-CO-2} (c43) (SEQ ID NO: 11), SYNC2_{SU-CO-3} (c43) (SEQ ID NO: 12), SYNC2_{SU-WT} (SEQ ID NO: 13), SYNC2_{SU-WT-2} (SEQ ID NO. 14), SYNC2_{SU-WT-3} (SEQ ID NO: 15), SYNC2_{SU-C43S} (SEQ ID NO: 16), SYNC2_{SU-C43S-2} (SEQ ID NO: 17), SYNC2_{SU-C43S-3} (SEQ ID NO: 18), RBL1 (SEQ ID NO: 19), RBL2 (SEQ ID NO: 20), RBL3 (SEQ ID NO: 21), RBLs4 (SEQ ID NO: 22), RBLs5 (SEQ ID NO: 23), or RBLs6 (SEQ ID NO: 24).

40. The method of claim 34, wherein the isolated syncytin 2 (SYNC2) polypeptide fragment comprises SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; or SEQ ID NO:24.


SEQUENCE LISTING (63 sequences)

SEQ ID NO: 1 - protein, 530 aa, synthetic construct
  REGION 1..530: note = MFSD2AWT
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 2 - protein, 530 aa, synthetic construct
  REGION 1..530: note = MFSD2ACO
MAKGEGAESGSAAGLLPTSILQSTERPAQVKKEPKKKKQQLSICNKLCYAVGGAPYQVTGCALGFFLQIYLLDVAQVGPFSASIILFVGRAWDAITDPLVGFCISKSSWTRLGRLMPWIIFSTPLAVIAYFLIWFVPDFPHGQTYWYLLFYCLFETLVTCFHVPYSALTMFISTEQSERDSATAYRMTVEVLGTVLGTAIQGQIVGQADTPCFQDLNSSTVASQSANHTHGTTSHRETQNAYLLAAGVIASIYVICAVILTLGVREQREPYEAQQSEPMSFFRGLRLVMSHGPYIKLIAGFLFTSLAFMLVEGNFALFCTYTLGFRNEFQNLLLAIMLSATLTIPIWQWFLTRFGKKTAVYVGISSAVPFLILVALMESNLIVTYVVAVAAGISVAAAFLLPWSMLPDVIDDFHLKQPHSHGTEPIFFSFYVFFTKFASGVSLGISTLSLDFAGYQTRGCSQPERVKFTLKMLVTMAPIVLILLGLLLFKLYPIDEERRRQNKKALQALRDEASSSGCSETDSTELASIL

SEQ ID NO: 3 - protein, 538 aa, synthetic construct
  REGION 1..538: note = SYNC2WT
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 4 - protein, 538 aa, synthetic construct
  REGION 1..538: note = SYNC2WT-C43S
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 5 - protein, 538 aa, synthetic construct
  REGION 1..538: note = SYNC2CO
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNLSAGRHPRNIQESPF

SEQ ID NO: 6 - protein, 538 aa, synthetic construct
  REGION 1..538: note = SYNC2CO-C43S
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNLSAGRHPRNIQESPF

SEQ ID NO: 7 - protein, 330 aa, synthetic construct
  REGION 1..330: note = SYNC2SU-CO
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAP

SEQ ID NO: 8 - protein, 475 aa, synthetic construct
  REGION 1..475: note = SYNC2SU-CO-2
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEG

SEQ ID NO: 9 - protein, 344 aa, synthetic construct
  REGION 1..344: note = SYNC2SU-CO-3
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSP

SEQ ID NO: 10 - protein, 330 aa, synthetic construct
  REGION 1..330: note = SYNC2SU-CO (C43)
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAP

SEQ ID NO: 11 - protein, 475 aa, synthetic construct
  REGION 1..475: note = SYNC2SU-CO-2 (C43)
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEG

SEQ ID NO: 12 - protein, 344 aa, synthetic construct
  REGION 1..344: note = SYNC2SU-CO-3 (C43)
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSP

SEQ ID NO: 13 - protein, 330 aa, synthetic construct
  REGION 1..330: note = SYNC2SU-WT-1
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAP

SEQ ID NO: 14 - protein, 475 aa, synthetic construct
  REGION 1..475: note = SYNC2SU-WT-2
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEG

SEQ ID NO: 15 - protein, 344 aa, synthetic construct
  REGION 1..344: note = SYNC2SU-WT-3
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSP

SEQ ID NO: 16 - protein, 330 aa, synthetic construct
  REGION 1..330: note = SYNC2SU-C43S
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAP

SEQ ID NO: 17 - protein, 475 aa, synthetic construct
  REGION 1..475: note = SYNC2SU-C43S-2
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEG

SEQ ID NO: 18 - protein, 344 aa, synthetic construct
  REGION 1..344: note = SYNC2SU-C43S-3
MGLLLLVLILTPSLAAYRHPDFPLLEKAQQLLQSTGSPYSTNSWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSP

SEQ ID NO: 19 - protein, 39 aa, synthetic construct
  REGION 1..39: note = RBL1
AELHISYRWDPNLKGLMRPANSLLSTVKQDFPDIRQKPP

SEQ ID NO: 20 - protein, 19 aa, synthetic construct
  REGION 1..19: note = RBL2
TYQTYTHNQFRHQPRFPKP

SEQ ID NO: 21 - protein, 31 aa, synthetic construct
  REGION 1..31: note = RBL3
TSYLGISAVSEFFGTSLTPLFHFHISTCLKT

SEQ ID NO: 22 - protein, 96 aa, synthetic construct
  REGION 1..96: note = RBLs4
TYQTYTHNQFRHQPRFPKPGNGTSYLGISAVSEFFGTSLTPLFHFHISTCLKTPGQGAELHISYRWDPNLKGLMRPANSLLSTVKQDFPDIRQKPP

SEQ ID NO: 23 - protein, 53 aa, synthetic construct
  REGION 1..53: note = RBLs5
TYQTYTHNQFRHQPRFPKPGNGTSYLGISAVSEFFGTSLTPLFHFHISTCLKT

SEQ ID NO: 24 - protein, 74 aa, synthetic construct
  REGION 1..74: note = RBLs6
TSYLGISAVSEFFGTSLTPLFHFHISTCLKTPGQGAELHISYRWDPNLKGLMRPANSLLSTVKQDFPDIRQKPP

SEQ ID NO: 25 - DNA, 1614 bp, synthetic construct
  misc_feature 1..1614: note = SYNC2WT DNA
  source 1..1614: mol_type = other DNA
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggcacctggaagtggttctcctgggtgctgcccctgaccggaccactggtgtctctgctgctgctgctgctgttcggcccttgtctgctgaacctgatcacacagtttgtgagctccagactgcaggccatcaagctgcagaccaatctgagcgccggcaggcacccaaggaacatccaggagtcccctttt

SEQ ID NO: 26 - DNA, 1614 bp, unidentified
  misc_feature 1..1614: note = SYNC2WT-C43S DNA
  source 1..1614: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggcacctggaagtggttctcctgggtgctgcccctgaccggaccactggtgtctctgctgctgctgctgctgttcggcccttgtctgctgaacctgatcacacagtttgtgagctccagactgcaggccatcaagctgcagaccaatctgagcgccggcaggcacccaaggaacatccaggagtcccctttt

SEQ ID NO: 27 - DNA, 1614 bp, unidentified
  misc_feature 1..1614: note = SYNC2CO DNA
  source 1..1614: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggcacctggaagtggttctcctgggtgctgcccttcatcggaccactggtgtctctgctgctgctgctgctgttcggcccttgtctgctgaacctgatcacacagtttgtgagctccagactgcaggccatcaagctgcagaccaatctgagcgccggcaggcacccaaggaacatccaggagtcccctttt

SEQ ID NO: 28 - DNA, 1614 bp, unidentified
  misc_feature 1..1614: note = SYNC2CO-C43S DNA
  source 1..1614: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggcacctggaagtggttctcctgggtgctgcccttcatcggaccactggtgtctctgctgctgctgctgctgttcggcccttgtctgctgaacctgatcacacagtttgtgagctccagactgcaggccatcaagctgcagaccaatctgagcgccggcaggcacccaaggaacatccaggagtcccctttt

SEQ ID NO: 29 - DNA, 990 bp, unidentified
  misc_feature 1..990: note = SYNC2SU-CO DNA
  source 1..990: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgcccca

SEQ ID NO: 30 - DNA, 1425 bp, unidentified
  misc_feature 1..1425: note = SYNC2SU-CO-2 DNA
  source 1..1425: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggc

SEQ ID NO: 31 - DNA, 1032 bp, unidentified
  misc_feature 1..1032: note = SYNC2SU-CO-3 DNA
  source 1..1032: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactcccca

SEQ ID NO: 32 - DNA, 990 bp, unidentified
  misc_feature 1..990: note = SYNC2SU-CO(C43)DNA
  source 1..990: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgcccca

SEQ ID NO: 33 - DNA, 1425 bp, unidentified
  misc_feature 1..1425: note = SYNC2SU-CO-2(C43)DNA
  source 1..1425: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggc

SEQ ID NO: 34 - DNA, 1032 bp, unidentified
  misc_feature 1..1032: note = SYNC2SU-CO-3(C43)DNA
  source 1..1032: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgaccgccaagagaaagaacggcacaaatgtgggcaccctgcccagcaccgtgtgcaatgtgacctttacagtggaccctaaccagcagacctaccagacctatacacacaatcagttccggcaccagcccagatttcctaagccacccaacatcaccttcccccagggcacactgctggataagtctacaaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactcccca

SEQ ID NO: 35 - DNA, 990 bp, unidentified
  misc_feature 1..990: note = SYNC2SU-WT DNA
  source 1..990: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgcccca

SEQ ID NO: 36 - DNA, 1425 bp, unidentified
  misc_feature 1..1425: note = SYNC2SU-WT-2
  source 1..1425: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggc

SEQ ID NO: 37 - DNA, 1032 bp, unidentified
  misc_feature 1..1032: note = SYNC2SU-WT-3
  source 1..1032: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaactgctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactcccca

SEQ ID NO: 38 - DNA, 990 bp, unidentified
  misc_feature 1..990: note = SYNC2SU-C43S DNA
  source 1..990: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgcccca

SEQ ID NO: 39 - DNA, 1425 bp, unidentified
  misc_feature 1..1425: note = SYNC2SU-C43S-2 DNA
  source 1..1425: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactccccactgcctcgggtgcggagagcaatccactttatcccactgctggccggcctgggaatcctggcaggaacaggaaccggaatcgcaggaatcaccaaggccagcctgacatatagccagctgtccaaggagatcgccaacaatatcgacacaatggccaaggccctgaccacaatgcaggagcagatcgattccctggccgcagtggtgctgcagaataggaggggcctggacatgctgaccgcagcacagggaggaatctgcctggccctggatgagaagtgctgtttctgggtgaaccagagcggcaaggtgcaggataatatccggcagctgctgaaccaggcctcctctctgcgggagagagccacacagggctggctgaattgggagggc

SEQ ID NO: 40 - DNA, 1032 bp, unidentified
  misc_feature 1..1032: note = SYNC2SU-C43S-3 DNA
  source 1..1032: mol_type = unassigned DNA; note = other
atgggcctgctgctgctggtgctgatcctgaccccaagcctggccgcatacaggcacccagacttccccctgctggagaaggcccagcagctgctgcagtctaccggcagcccttattccacaaacagctggctgtgcaccagctcctctaccgagacaccaggcacagcctaccctgcctctccacgcgagtggaccagcatcgaggccgagctgcacatctcctatagatgggatccaaatctgaagggcctgatgcgccccgccaactctctgctgagcaccgtgaagcaggacttccctgatatccggcagaagccccctatcttcggcccaatctttacaaacatcaatctgatgggcatcgccccaatctgcgtgatggccaagagaaagaacggcacaaatgtgggcaccctgcccagcaccgtgtgcaatgtgacctttacagtggacagcaaccagcagacctaccagacctatacacacaatcagttccggcaccagcccagatttcctaagccacccaacatcaccttcccccagggcacactgctggataagtctagcaggttttgccagggccgcccaagctcctgtagcacccggaatttctggtttagacccgccgactacaatcagtgcctgcagatctctaacctgtctagcacagccgagtgggtgctgctggatcagaccaggaactccctgttctgggagaataagacaaagggcgccaaccagtctcagaccccttgcgtgcaggtgctggccggaatgacaatcgccacctcctacctgggcatctccgccgtgtctgagttctttggcacatctctgacccccctgttccactttcacatcagcacctgcctgaagacacagggcgccttttatatctgcggccagtccatccaccagtgtctgccttctaactggaccggcacatgtaccatcggctacgtgacccctgacatcttcatcgccccaggcaatctgagcctgcctatcccaatctatggcaactcccca

SEQ ID NO: 41 - protein, 530 aa, synthetic construct; identical to SEQ ID NO: 1
  REGION 1..530: note = MFSD2A_Human

SEQ ID NO: 42 - protein, 529 aa, synthetic construct
  REGION 1..529: note = mfsd2a_Mouse
MAKGEGAESGSAAGLLPTSILQASERPVQVKKEPKKKQQLSICNKLCYAVGGAPYQLTGCALGFFLQIYLLDVAKVEPLPASIILFVGRAWDAFTDPLVGFCISKSSWTRLGRLMPWIIFSTPLAIIAYFLIWFVPDFPSGTFLWYLLFYCLFETLVTCFHVPYSALTMFISTEQSERDSATAYRMTVEVLGTVIGTAIQGQIVGQAKAPCLQDQNGSVVVSEVANRTQSTASLKDTQNAYLLAAGIIASIYVLCAFILILGVREQRELYESQQAESMPFFQGLRLVMGHGPYVKLIAGFLFTSLAFMLVEGNFALFCTYTLDFRNEFQNLLLAIMLSATFTIPIWQWFLTRFGKKTAVYIGISSAVPFLILVALMERNLIVTYVVAVAAGVSVAAAFLLPWSMLPDVIDDFHLKHPHSPGTEPIFFSFYVFFTKFASGVSLGVSTLSLDFANYQRQGCSQPEQVKFTLKMLVTMAPIILILLGLLLFKLYPIDEEKRRQNKKALQALREEASSSGCSDTDSTELASIL

SEQ ID NO: 43 - protein, 528 aa, synthetic construct
  REGION 1..528: note = mfsd2a_Elefant
MAKGEGAEGGSAAGLLPTSILQTGERPAHVKKEPKKKQQLSICNKLCYAVGGAPYQTGCALGFFLQIYLLMLSKVDPFSASIILFVGRAWDAITDPLVGFCISKSSWTRLGRLMPWIIFSTPPAIIAYFLIWFVPDFPQGQALWYLLFYCLFETLVTCFHVPYSALTMFISTEQSERDSATAYRMTVEVLGTVLGTAIQGQIVGQVDTPCLQDPNDFAMASEGVNRTHSTTSLKETQNAYLLAAGVISSLYVICAVILTLGVREQREPYKTQQTEHTSFFRGLRLVMSHGPYVRLIAGFLFTSLAFMLVEGNFALFCTYTLGFRNEFQNLLLAVMLSATFTIPVWQWFLTRFGKKTAVYIGISSAVPFLILVALMDSNLIVTYVVAVAAGISVAAAFLLPWSMLPDVIDDFHLKQPHSHGTEPIFFSFYVFFTKFASGVSLGISTLSLDFAGYQTRGCSQPARVKFTLKMLVTMAPIVLILLGLLLFKLYPIDEEKRRQNKKALQALRDEAGSSGCSDTDSTELASIL

SEQ ID NO: 44 - protein, 529 aa, synthetic construct
  REGION 1..529: note = Mfsd2a_Dog
MAKGEGAESGSAAGLLPTGILPAAERPAQVKKEPKKKQQLSICNKLCYAVGGAPYQVTGCALGFFLQIYLLDVAQVEPFFASIILFVGRAWDAFTDPLVGFCISKSSWTRLGRLMPWIIFSTPLAIIAYFLIWFVPDFPRGQALWYLLFYCLFETLVTCFHVPYSALTMFISTEQSERDSATAYRMTVEVLGTVLGTAIQGQIVGQADTPCLQDPSDSALAMEGANHTQSTTSLKETQNAYLLAAGVIASIYVICAVILTLGVREQREPYETQQAEPMSFFRGLRLVMSHGPYVKLIAGFLFTSLAFMLVEGNFALFCTYTLGFRNEFQNLLLAIMLSATFTIPIWQWFLTRFGKKTAVYVGISSAVPFLILVAFMESNLIVTYVVAVAAGISVAAAFLLPWSMLPDVIDDFHLKQPQSHGTEPIFFSFYVFFTKFASGVSLGISTLSLDFAGYQTRGCSQPARVKFTLKLLVTIAPIVLILLGLLLFKLYPIDEEKRRQNKKALQALREEASSSGCSDTDSTELASIL

SEQ ID NO: 45 - protein, 523 aa, synthetic construct
  REGION 1..523: note = mfsd2a_cow
MAKGEGAESGSAAGLLPTGILQAGERPVQVKEPKKKKQLSICNKLCYAVGGAPYQVTGCALGFFLQIYLLDVAQVDPFSASIILFVGRAWDAITDPLVGFCISKSPWTRLGRLMPWITFSTPLAIIAYFLIWFVPDFHQGQTLWYLLFYCLFETLVTCFHVPYSALTMFISTEQSERDSATAYRMTVEVLGTVLGTAIQGQIVGQADSPCIPDANASTVNRTQSSTSIKETQNAYLLAAGVIASIYVICAVILILGVREQRESYETQQTKQMPFFRGLRLVMSHGPYIKLIAGFLFTSLAFMLVEGNFALFCTYTLGFRNEFQNLLLAIMFSATVTIPIWQWFLTRFGKKTAVYIGISSAVPFLILVALMESNLIVTYVVAVAAGISVAAAFLLPWSMLPDVIDDFHLKQPHIHGTEPIFFSFYVFFTKFASGVSLGISTLSLDFTGYQTRGCSQPARVKFTLKMLVTMAPIVLILIGLLLFKLYPIDEEKRRQNKKALQALREEASSSGCSDTDSTELASIL

SEQ ID NO: 46 - protein, 528 aa, synthetic construct
  REGION 1..528: note = mfsd2a_Armadillo
MAKGEGAESGSAAGLLPTGILQAGERPARVKKEPKKKQQLSICNKLCYAVGGAPYQVTGCALGFFLQIYLLDVAQVNPFSASIILFVGRAWDAVTDPLVGFCISKTSWTRLGRLMPWIIFSTPLAVITYFLIWFVPDFPRHQALWYLLFYCLFETLVTCFHVPYSALTMFISTEQSERDSATAYRMTVEVLGTVLGTAIQGQIVGQADTPCLQSPNDSTVTLEGANRTHSAISLKKTQNAYLLAAGVIASIYVICAVILTLGVREQREPYETQKEPMSFFRGLRLVMSHGPYVKLIAGFLFTSLAFMLVEGNFALFCTYTLGFRNEFQNLLLVIMLSATFTIPFWQWFLTRFGKKTAVYIGISSAVPFLMLVALMKSNLIVTYVVAVAAGISVAAAFLLPWSMLPDVVDDFHLKQPHSHGTEPIFFSFYVFFTKFASGVSLGISTLSLDFAGYQTRGCSQPKLVRLTLKMLVTMAPIVLILLGLLLFKLYPIDEEKRRQNRKALQALREEASSSGCSDTDSTELASIL

SEQ ID NO: 47 - protein, 503 aa, synthetic construct
  REGION 1..503: note = MFSD2B_Human
MAAPPAPAAKGSQPEPHAPEPGPGSAKRGREDSRAGRLSFCTKVCYGIGGVPNQIASSATAFYLQLFLLDIAQIPAAQVSLVLFGGKVSGAAADPVAGFFINRSQRTGSGRLMPWVLGCTPFIALAYFFLWFLPPFTSLRGLWYTTFYCLFQALATFFQVPYTALTMLLTPCPRERDSATAYRMTVEMAGTLMGATVHGLIVSGAHRPHRCEATATPGPVTVSPNAAHLYCIAAAVVVVTYPVCISLLCLGVKERPDPSAPASGPGLSFLAGLSLTTRHPPYLKLVISFLFISAAVQVEQSYLVLFCTHASQLHDHVQGLVLTVLVSAVLSTPLWEWVLQRFGKKTSAFGIFAMVPFAILLAAVPTAPVAYVVAFVSGVSIAVSLLLPWSMLPDVVDDFQLQHRHGPGLETIFYSSYVFFTKLSGACALGISTLSLEFSGYKAGVCKQAEEVVVTLKVLIGAVPTCMILAGLCILMVGSTPKTPSRDASSRLSLRRRTSYSLA

SEQ ID NO: 48 - protein, 494 aa, synthetic construct
  REGION 1..494: note = mfsd2b_mouse
MSVPHGPTPAPVAEPHTQEPGSDKRDGRLSVCTKVCYGIGGVPNQVASSASAFYLQLFLLDVAQIPAAQVSLALFGGKVSGAVADPVAGFFINKSRRTGSGRLMPWALGCMPLIALAYFFLWFLPPFTSLRGLWYTSFYCLFQALATFFQVPYTALTMILTPSPRERDSATAYRMTMEMAGTLMGATVHGLIVSSAHGSQRCEDTVHPRSPAVSPDVARLYCIAAAVVALTYPVCGSLLCLGVKEQPDTSAPASGQGLNFFTGLAITSQHPPYLSLVVSFLFISAAVQVEQSYLVLFCTHASKLQDHVQNLVLIILVSAVLSTPLWEWVLQRFGKKTSAFGICVMVPFSILLAAVPSAPVAYVVAFVSGVSIAVSLLLPWSMLPDVVDDFQLQHRCGPGVETIFYSSYVFFTKLSGAGALGISTLSLEFAGYEAGACQQAEEVVVTLKVLIGAVPTCMILIGLCILLVGPTPKMPRQDTSSQLSLRRRTSYSLA

SEQ ID NO: 49 - protein, 490 aa, synthetic construct
  REGION 1..490: note = mfsd2b_Elefant
MPNIVLATGDAMLQCLPQDSRAGRLSFCTKLCYGIGGIPNQVASSATAFYLQLFLLDVAQIPAAQVSLVLFGGKVSGAAADPVAGFFINRSRRTGSGRLMPWVLGCTPFIALAYFFLWFLPPFTNLRGVWYMTFYCLFQALATFFQVPYTALTMLLTPNPRERDSATAYRITMEMAGTLMGATVHGLIVSRAHGPHRCQEAEIPVPDGVSPDAMRLYSIAAAVVAVTYPVCSGLLRLGVKERPDPSTPASGQGLSFLAGLGLTVRHPPYLKLVVSFLFISAAVQVEQNYLVLFCTHASRLHDHVQRLVLTILISAVLSTPLWEWVLHRFGKKTPAFGICVMVPFAILLAAVPVAPVAYVVAFVSGVSIAVSLLLPWSMLPDVVDDFQLQHQHGPGLETIFYSSYVFFTKLSGASALGISTLSLEFVGYKAGACEQTEEVVVTLKVLIGAVPTCMILTGLCILLVSRTPKVPSQDSSRQLSLRRRTSYSLA

SEQ ID NO: 50 - protein, 453 aa, synthetic construct
  REGION 1..453: note = mfsd2b_Dog
MPNQVASSATAFYLQLFLLDVAQIPAAQVSLVLFGGKVSGAAADPVAGFFINRSSRTGSGRLMPWVLGCTPFIAVAYFLLWFLPPFSSLRGLWYTAFYCVFQALATSLQVPYAALTMRLTPCPRERDSATAYRMTMEMAGTLLGAAVHGLLVSGAHRPHPCEDAAPPGPQTDSADATRLYSIAAAVVAMMYPVCSGLLCLGVKERPDSSAPASGQGLSFLAGLGLTVRHPPYLKLVISFLFISAAIQVEQSYLVLFCTHASRLHDHVQGVVLTILVSAVLSTPLWEWVLQRFGKKTSAFGICVMVPFAILLAAVPTAPVAYVVAFVSGLSIAVSLLLPWSMLPDVVDDFQLQHQHSPGLETIFYSSYVFFTKLSGAGALGISTLSLEFAGYEAGACKQAEQVVVTLKVLIGAVPTCMILTGLCILMAGPAPKVPSQDPSRRLSLQRRTSYSLA

SEQ ID NO: 51 - protein, 480 aa, synthetic construct
  REGION 1..480: note = mfsd2b_Cow
MGAELLCLPQDSGAGRLSFYRKLCYGIGGVPNQVASSAIAFYLQLFLLDVAQIPAAQVSLVLFGGKVSGAAADPLAGFLINRSRRTGSGRLMPWVLGCMPFIALAYFFLWFLPPFTTLRGLWYTTLYCLFQALATFFQVPYTALTMLLTPNPKERDSATAYRMTLEMAGTLMGATVHGLIVSGAHGSHRCKEDTLPGEGAVSPNATRLYFIAATVVALTYPVCSTLLYLGVKERSDPSTPASGQGLGFLAGLGLTVRHRPYLNLVISFLFISAAVQVEQSYLVLFCTHASQLQDHVQGMVLTILVSAVLSTPMWEWVLQRFGKRMSALGICAMVPFAILLAAVPMVPVAYVVAFVSGLSIAVSLLLPWSMLPDVVDDFQLQHQHGPGLETIFYSSYVFFTKLSGAGALGISTLSLDFAGYESGACRQSEQVVVTLKVLIGAVPTSMILIGLCILMVGPTPKVPSRANSRSLGRRTSYTLA

SEQ ID NO: 52 - protein, 505 aa, synthetic construct
  REGION 1..505: note = mfsd2b_Armadillo
MRPGQDAGRGGTVRLLRSQRLHPRERPGRPRGQDSGAGRLSFCTKLCYGIGGVPNQMASSATAFYLQLFLLDVAQIPAAQVSLVLCGGKVSGAAADPVAGFLINRSRRTGSGHLMPWVLGCTPLAALAYFLLWLLPPAASLRGLWYTAFYCLFQALSTALHVPYAALTMLLTECPMERDSATAYRMTMEMAGTLAGAAAHGLLVAGAHQPRGCHEAALPGPGGVSPHAARLYSIAAAVVAAAYPVCSCLLCLGVKERPEPPGPDSGPGLGFLAGLGLTARHPPYLRLVVSFLLISAAVQVEQSYLVLFCTHAAQLHGHVQGLVLTVLVSAVLSTPLWEWVLQRFGKMTSXXGLCTGAPGVRLTWSLPRLLTGWWPSRGNPGXLCPAPPPPRSMLPDVVDGFQRQHPHGPGLETIFYSSYVFFTKLSGAGALGISTLSLEFAGYKAGACEQVEEVAVTLKVLISAVPTCMILAGLCVLRAGPRPEVPGPASARQLSLRRRTSYSLA

SEQ ID NO: 53 - protein, 538 aa, synthetic construct; identical to SEQ ID NO: 3
  REGION 1..538: note = human SYNC2WT

SEQ ID NO: 54 - protein, 538 aa, synthetic construct
  REGION 1..538: note = Sync2_Chimpazee
MGLLLLVLILTPSLAAYRHPDFPLLEKAQQLLQSTGSPYSTNCWLCTSSSTETPGTAYPASPREWTSIEAELHISYRWDPNLKGLMRPANSLLSMVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSQLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 55 - protein, 538 aa, synthetic construct
  REGION 1..538: note = Sync2_Gorilla
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVTAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQLIHQCLPSNWTGTCTIGYVTPDIFIAPGNLSLPIPIYGNSQLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRRPRNIQESPF

SEQ ID NO: 56 - protein, 538 aa, synthetic construct
  REGION 1..538: note = Sync2_Orangutan
MGLLLLVLILTPLLAAHRHPDFPLLEKAQQLLQSTGSPYSTNCWLCTSSSTETPGTAYPASPREWTSIEAELHISYHWDPNLKGLMRPANSLLSTVKQDFPDIRQKPPIFGPIFTNINLMGIAPICVTAKRKNGTNVGTLPSTVCNVTFTVDPNQQTYQTYTHKQFLHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNISLPIPIYGNSQLPRVRRAIHFIPLLAGLGIIAGTGTGIAGITKASLTYSQLSKEIAKNIDTMAKALTTVQEQIDSLAAVVLQNRRGLDMLTAAQGGICLALDEKCCFWVNQSGKVQDNIRQLLNQASSLRERATQGWLNWEGTWKWFSWVLPFTGPLVSLLLLLLFGPCLLNLITQFVLSRLQAIKLQTNLSAGCRPHNIQESPF

SEQ ID NO: 57 - protein, 538 aa, synthetic construct
  REGION 1..538: note = Sync2_Gibbon
MGLLLLVLILTPLLAAYRHPDFPLLEKAQQLLQSTGSPYSTNCWLCTSSSTETPGTAYPASPREWTSIEAELHISYQWDPNLKGLMRPANSLLSTVKQDFPDIRQKPPIFGPIFTNINLMGIAPICVTAKRKNGTNVGTLPSTVCNVTFTVDPNQQTYQTYTHNQFRHQPRFPKPPNITFPQGTLLDKSTRFCQGRPSSCSTRNFWFRPADYNQCLQISNLSSTAEWVLLDQTRNSLFWENKTKGANQSQTPCVQVLAGMTIATSYLGISAVSEFFGNSLTPLFHFHISTCLKTQGAFYICGQSIHQCLPSNWTGTCTIGYVTPDIFIAPGNLSLPIPIYGKSQLPRVRRAIHFIPLLAGLGILAGTGTGIAGITKASLTYSQLSKEIANNIDTMAKTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNLSAGRRPRTIQESPF

SEQ ID NO: 58 - protein, 537 aa, synthetic construct
  REGION 1..537: note = Sync2_Macaques
MGLLLLVLILTPLLAAYRHPDFPLLEKAQQLLQSTGSPYSTNCWLCTSSSTETPGTAYPASPREWTSIEAELHISYQWDPNLKGLMTPANSLLSTVKQDFPDIRQKPPIFGPIFTNINLMGKAPICVTAKRKNGTNVGTLPSTVCNVTFTVDPNQQTYQTYTHNQFRHQPRFPKPPNITFPQGTLLDKSTQFCQGRPSSCRTRNFWFRPADYNQCLQIPKLSSPAEWVLLDQTRNSLFWENKTKGANQSQTPCIQVLAGMTIATSYLGISAVSEFFGTSLTPLFHFHISTCLKTQGAFYICGQSIHQCLPTNWTGTCTIGYVTPDIFIAPGNLSLPIPIYGNSQLPRVRRAIHFIPLLAGLGILAGTGTGIAGITKASFTYSQLSKEIAKNIDTMAKTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNGAGCRPRNIQESPF

SEQ ID NO: 59 - protein, 537 aa, synthetic construct
  REGION 1..537: note = Sync2_Baboon
MGLLLLVLILTPLLAAYRHPDFPLLEKAQQLLQSTGSPYSTNCWLCTSSSTEMPGTAYPASPREWTSIEAELHISYQWDPNLKGLMTPANSLLSTVKQDFPDIRQKPPIFGPIFTNINLMGKAPICVTAKRKNGTNVGTLPSTVCNVTFTVDPNQQTYQTYTHNQFRHQPRFPKPPNITFPQGTLLDKSTQFCQGRPSSCRTRNFWFRPADYNQCLQIPNLSSPAEWVLLDQTRNSLFWENKTKGANQSQTPCIQVLAGMTIATSYLGISAVSEFFGTSLTPLFHFHISTCLKTQGAFYICGQSIHQCLPTNWTGTCTIGYVTPDIFIAPGNLSFPIPIYGNSQLPRVRRAIHFIPLLAGLGILAGTGTGIAGITKASFTYSQLSKEIAKNIDTMAKTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNGAGCRPRNIQESTF

SEQ ID NO: 60 - protein, 537 aa, synthetic construct
  REGION 1..537: note = Sync2_green_monkey
MGLLLLVLILTPLLAAYRHPDFSLLEKAQQLLQSTGSPYSTNCWLCTRSSTETPGTAYPASPREWTSIEAELHISYQWDPNLKGLMTPANSLLSTVKQDFPDIRQKPPIFGPIFTNINLMGKAPICVTAKRKNGTNVGTLPSTVCNVTFTVDPHQQTYQTYTHNQFRHQPRFPKPPNITFPQGTLLDKSTQFCQGRPSSCRTRNFWFRPADYNQCLQIPNLSSPAEWVLLDQTRNSLFWENKTKGANQSQTPCIQVLAGMTIATSYLGISAVSEFFGTSLTPLFHFHISTCLKTQGAFYICGQSIHQCLPTNWTGTCTIGYVTPDIFIAPGNLSLPIPIYGNSQLPRVRRAIHFIPLLAGLGILAGTGTGIAGITKASFTYSQLSKEIAKNIDTLAKTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNSAGCRPRNIQESPF

SEQ ID NO: 61 - protein, 537 aa, synthetic construct
  REGION 1..537: note = Sync2_Golden_s.nosed
MGLLLLVLILTPLLAAYRHPDFPLLEKAQQLLQSTGSPYSTSCWLCTSSSTETPGTAYPASPREWTSIEAELHISYQWDPNLKGLMTPANSLLSTVQQDFPDIRQKPPIFGPIFTNINLMGKAPICVTAKRKNGTNVGTLPSTVCNVTFTVDPNQQTYQTYTHNQFRHQPRFPKPPNITFPQGTLLDKSTQFCQGRPSSCRTRNFWFRPADYNQCLQIPNLSSPAEWVLLDQTRNSLFWENKTKGANQSQTPCVQVLAGMTIATSYLGISAVSEFFGTSLTPLFHFHISTCLKTQGAFYICGQSIHQCLPTNWTGTCTIGYVTPDIFIAPGNLSLPIPIYGNSQLPRVRRAIHFIPLLAGLGILAGTGTGIAGITKASFTYSQLSKEIAKNIDTMAKTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNSARCRPRNIQESPF

SEQ ID NO: 62 - protein, 537 aa, synthetic construct
  REGION 1..537: note = Sync2_Angolan_colubus
MGLLLLVLILTPLLAAYRHPDFPLLEKAQQLLQSTRSPYSTNCWLCTSSSTKTPGTAYPASPREWTSIEAELHISYQWDPNLKGLMTPANSLLSTVKQDFPDIRQKPPIFGPIFTNINLMGKAPICVTAKRKNGTNVGTLPSTVCNVTFTVDPNQQTYQTYTHNQFRHQPRFPKPPNITFPQGTLLDKSTQFCQGRPSSCRTRNFWFRPADYNQCLQIPNLSSPAEWVLLDQTRNSLFWENKTNGANQSQTPCVQVLAGMTIATSYLGISAVSEFFGTSLTPLFHFHISTCLKTQGAFYICGQSIHQCLPTNWTGTCTIGYVTPDIFIAPGNLSLPIPIYGNSQLPRVRRAIHFIPLLAGLGILAGTGTGIAGITKASFTYSQLSKEIAKNIDTMAQTLTTVQEQIDSLAAVVLQNRRGLDMLTAAQGGICLALDEKCCFWVNQSGKVQDNIRQLLNQASSLRERATQGWLNWEGTWKWFSWVLPFIGPLVSLLLLLLFGPCLLNLITQFVSSRLQAIKLQTNSARCRPRNIQESPF

SEQ ID NO: 63 - protein, 538 aa, synthetic construct
  REGION 1..538: note = Sync2_marmoset
MGLLLLLLILTPLLAAYRHPDFRLLEKAQQLLQSTGSPYSTNCWLCTSSSTKTPGRAYPASSREWTTIEAELHISYQWDPNLKGLIKPANSLLSKVKQDFPDIRKEPPIFGPIFTNVNLIGIAPICVTAKRKDGTNVGTLPSTVCNVTLTVDPNQQTYQKYAHNQFHHQPRFPKPPNITFPQGTLLDKSTRFCQGRPSSCSTRNFWFQPADYNQCLQIPNLSSTAEWVLLDQTRNSLFWENKTKGANQSQTPCVQVLAGMTIATSYLSISAVSEFSGTSVTSLFSFHISTCLKTQGAFYICGQSIHQCLPTNWTGTCTIGYVSPDIFIAPGNLSLPIPIYGNSHFPRVRRAIHLIPLLVGLGIVGSAGTGIAGIAKASFTYSQLSKEIANNIEAMAKTLTTVQEQIDSLAAVVLQNRRGLDMLTAAQGGICLALEEKCCFWVNQSGKVQDNIRQLLNRASTLQEQATQGWLSWEGTWKWFSWVLPFTGPLVSLLLLLLFGPCLLNLITQFVSSRLQATKLQMKLNKRVRPRNSQESPF